(19) United States
(12) Patent Application Publication
(10) Pub. No.: US 2006/0275810 A1
(43) Pub. Date: Dec. 7, 2006

(54) Focused microarray and methods of diagnosing chemotherapeutic drug resistance in a cancer cell

(76) Inventors: Elias Georges, Laval (CA); Claudia Boucher, Notre-Dame de l'lle Perrot (CA); Anne-Marie Bonneau, Laval (CA)

Correspondence Address: WILMER CUTLER PICKERING HALE AND DORR LLP, 60 STATE STREET, BOSTON, MA, 02109, US

(21) Appl. No.: 11/443,856

(22) Filed: May 30, 2006

Related U.S. Application Data
(60) Provisional application No. 60/685,284, filed on May 27, 2005.

(51) Int. Cl. C12Q 1/68; C12M 1/34

(52) U.S. Cl. 435/6; 435/287.2; 977/924

(57) ABSTRACT

Disclosed are methods for diagnosing chemotherapeutic drug resistance in a cancer cell sample by detecting an increase in the levels of expression of marker genes in the cancer cell sample as compared to the levels of expression of the same marker genes in a chemotherapeutic drug-sensitive cancer cell of the same tissue type. Also disclosed is a focused microarray device for diagnosis of chemotherapeutic drug resistance in cancer cells.

### FIELD OF THE INVENTION

The present invention relates generally to the field of medicine. More specifically, the invention pertains to a device and methods for detecting the development of chemotherapeutic drug resistance in cancer cells.

### BACKGROUND OF THE INVENTION

A commonly used treatment for most cancer diseases is the administration of compounds to kill cancer cells, e.g., chemotherapeutics. The drugs exploited for such purposes must selectively inhibit the survival of the diseased cancer cell in order to eliminate the cancer. Unfortunately, conventional chemotherapies also disrupt the biochemical machinery of normal cells as well, producing significant adverse effects on the patient. Consequently, it is important that a chemotherapy treatment regime maximize the effectiveness of the drugs against the cancer cells, while reducing the patient's exposure to the chemotherapy regime.

Although chemotherapeutic drugs have presented clinicians with a powerful tool against neoplasms, many cancer cells become resistant to a particular course of treatment (termed “chemotherapeutic drug resistance”). In the clinical setting, the development of chemotherapeutic drug-resistant neoplasms is the principal reason for treatment failure and mortality in cancer patients (see Gottesman, Ann. Rev. Med. 53: 615-627, 2000). Generally, chemotherapeutic drug resistance is the point at which a particular drug or class of drugs no longer effectively kills a subset of cancer cells within a patient. The general mechanisms of chemotherapeutic drug resistance, though still relatively unknown, involve the aberrant expression of several classes of genes controlling drug metabolism, drug transport, and apoptosis. Such genes act to render the treatment ineffective against the target cell by reducing the dosage of drug within a cancer cell, allowing the cell to survive the treatment and propagate itself.

Although certain mechanisms of drug resistance have been elucidated, chemotherapeutic drug resistance is likely to be a multifactorial trait that involves many different genes acting in different cancer cell types. The diagnosis of drug resistance is confounded by situations in which more than one gene acts to produce resistance to a particular drug or class of drugs. In these situations, genetic variability may create drug resistance to the same drug in different cancer cells through completely different mechanisms. Thus, what are needed are materials and methods optimized for diagnosing chemotherapeutic drug resistance using a plurality of cell markers tailored to the identification of complex drug resistance.

Microarray technology has been used to analyze the expression of a large number of drug resistance cell markers in a single diagnostic experiment. This technology provides a platform that allows for rapid quantification of gene products, e.g., mRNA and protein. In addition, most microarrays presently available contain thousands of genes representing a large cross-section of the genome of a particular cell or tissue (termed “pangenomic microarrays”). Pangenomic microarrays have provided scientific researchers with a powerful tool to analyze entire tissue expression profiles at a particular moment in time. As a result of their ease of use and the volume of information they generate, microarrays have become the “workhorses” for genomic research and have been used to elucidate expression differences in gene expression between tissues and cell types, as well as differences occurring throughout development.

Unfortunately, the pangenomic nature of many microarrays necessarily means that a significant amount of information will be generated that has little diagnostic significance in determining the onset of chemotherapeutic drug resistance in a particular neoplasm. More importantly, many of the data points on a pangenomic microarray may be detrimental to the usefulness of a clinical evaluation of chemotherapeutic drug resistance due to the potential misinterpretation of the expression profile by the clinician. Focused microarrays contain genes whose relationship to a particular disease or disorder has been established. In general, focused microarrays are used to analyze a limited number of genes, rather than an entire genome (van 't Veer, et al., (2002) Nature. 415(6871): 530-6), and in most cases are based on prior Proteomics analyses.

### SUMMARY OF THE INVENTION

By analyzing the expression level of several drug metabolism genes in a neoplasm in a single experiment, the total number of drugs to which a neoplasm is resistant can be determined while accounting for the genetic variability of drug resistance in individual cancer cells. The invention is based in part upon the discovery that certain genes are overexpressed at the mRNA and protein level in neoplasms that have developed chemotherapeutic drug resistance. These gene expression patterns are therefore diagnostic of the presence of chemotherapeutic drug resistance. This discovery has been exploited to provide an invention that allows for the use of capture probes to determine the expression of a multiplicity of select cell markers in a neoplasm in order to diagnose chemotherapeutic resistance in the neoplasm.

In one aspect, the invention provides a method of diagnosing chemotherapeutic drug resistance in a cancer cell sample using a focused microarray. The focused microarray has a plurality of nucleic acid capture probes that are each complementary to a marker gene from the group consisting of Pgp 1, BCRP, P53, annexin-1, UCHL-1, ezrin, HnRNP, E-FABP, “similar to stratifin”, HSP27, SOD, γ-actin, vimentin, HSC70, galectin-1, prosolin, β-tubulin, GST-Π, α-enolase, HSP90, HSP60, nucleophosmin, PDI/ER-60 precursor, FAS, Rad23 homolog β, α-tubulin, MRP1, keratin type II, ATP synthase δ, tropomyosin 2β, prohibitin, calumenin, 5C5-2, SLC9A3R1, pyrophosphatase inorganic, DADEH1, EIF-4B, APRT, LRP/MVP, MB-COMT, EF2, PDI, BIP, and thioredoxine peroxidase 1. The method entails detecting a level of expression in the cancer cell sample of a plurality of marker genes complementary to the plurality of nucleic acid capture probes on the focused microarray, and then comparing the level of expression of the plurality of marker genes in the cancer cell sample to the level of expression of the plurality of marker genes in a non-drug-resistant cancer cell of the same tissue type. If the level of expression of one or more of the plurality of marker genes in the cancer cell sample is greater than the level of expression of the same marker gene(s) in the non-drug-resistant cancer cell of the same tissue type, then cancer cell sample is chemotherapeutic drug-resistant. In this aspect, the microarray does not include nucleic acid capture probes complementary to cellular marker genes from the group consisting of Ki67, estrogen receptor α, estrogen receptor β, Bcl-2, cathepsin β, cathepsin δ, keratin 19, topoisomerase type II α, P53, and GAPDH.

In certain embodiments, the cancer cell is drug-resistant if the level of expression of at least two or more of the plurality of marker genes detected in the cancer cell is greater than the level of expression of the same marker gene(s) in the non-drug-resistant cancer cell of the same tissue type. In other embodiments, the cancer cell is drug-resistant if the level of expression of at least three or more of the plurality of marker genes detected in the cancer cell is greater than the level of expression of the same marker gene(s) in the non-drug resistant cancer cell of the same tissue type. In particular embodiments, drug-resistance is indicated if the level of expression of at least four or more of the plurality of marker genes detected in the cancer cell is greater than the level of expression of the same marker gene(s) in the non-drug resistant cancer cell of the same tissue type.

In certain embodiments, the focused microarray has a plurality of nucleic acid capture probes complementary to cell markers from the group consisting of annexin-1, galectin-1, α-enolase, MRP1, PDI/ER-60 precursor, keratin type II, calumenin, prohibitin, and Pgp 1. In particular embodiments, the plurality of nucleic acid capture probes can be at least two. In other particular embodiments, the plurality of nucleic acid capture probes can be at least three. In more particular embodiments, the plurality of nucleic acid capture probes can be at least four. In still more embodiments, the plurality of nucleic acid capture probes can be at least five. In particular embodiments, chemotherapeutic drug resistance is detected when the level of expression of annexin-1 is greater in a drug-resistant breast cancer cell than in a non-resistant breast cancer cell. In more particular embodiments, chemotherapeutic drug resistance is detected when the level of expression of keratin type II is greater in a drug-resistant lung cancer cell than in a non-resistant lung cancer cell. In still more particular embodiments, chemotherapeutic drug resistance is detected when the level of expression of annexin-1 is greater in a drug-resistant ovarian cancer cell than in a non-resistant ovarian cancer cell.

In another aspect, the invention provides a method of diagnosing chemotherapeutic drug resistance in a cancer cell sample using a focused microarray. The focused microarray has a plurality of at least five nucleic acid capture probes that are complementary to marker genes from the group consisting of Pgp 1, BCRP, P53, annexin-1, UCHL-1, ezrin, HnRNP, E-FABP, “similar to stratifin”, HSP27, SOD, γ-actin, vimentin, HSC70, galectin-1, prosolin, β-tubulin, GST-Π, α-enolase, HSP90, HSP60, nucleophosmin, PDI/ER-60 precursor, FAS, Rad23 homolog β, α-tubulin, MRP1, keratin type II, ATP synthase δ, tropomyosin 2β, calumenin, prohibitin, 5C5-2, SLC9A3R1, pyrophosphatase inorganic, DADEH1, EIF-4B, APRT, LRP/MVP, MB-COMT, EF2, PDI, BIP, and thioredoxine peroxidase 1. The method entails detecting a level of expression in the cancer cell sample of a plurality of marker genes complementary to the plurality of nucleic acid capture probes on the focused microarray, and then comparing the level of expression of the plurality of marker genes in the cancer cell sample to the level of expression of the plurality of marker genes in a non-drug-resistant cancer cell of the same tissue type. If the level of expression of one or more of the plurality of marker genes in the cancer cell sample is greater than the level of expression of the same marker gene(s) in the non-drug-resistant cancer cell of the same tissue type, then the cancer cell sample is likely to be resistant to chemotherapeutic treatment.

In certain embodiments, the microarray has a plurality of nucleic acid capture probes from the group consisting of annexin-1, galectin-1, α-enolase, MRP1, PDI/ER-60 precursor, keratin type II, calumenin, prohibitin, and Pgp 1. In particular embodiments, the plurality of nucleic acid capture probes is at least six. In other embodiments, the plurality of nucleic acid capture probes is at least seven. In still other embodiments, the plurality of nucleic acid capture probes is at least eight.

In further embodiments, the cancer cell is drug-resistant if the level of expression of two or more of the plurality of marker genes in the cancer cell sample is greater than the level of expression of the same marker gene(s) in the non-drug-resistant cancer cell of the same tissue type. In additional embodiments, the cancer cell is drug-resistant if the level of expression of three or more of the plurality of marker genes in the cancer cell sample is greater than the level of expression of the same marker gene(s) in the non-drug-resistant cancer cell of the same tissue type. In yet other embodiments, the cancer cell is drug-resistant if the level of expression of four or more of the plurality of marker genes in the cancer cell sample is greater than the level of expression of the same marker gene(s) in the non-drug-resistant cancer cell of the same tissue type.

In some embodiments, the level of expression of annexin-1 is detected and the cancer cell is from breast tissue. In other embodiments, the level of expression of keratin type II is detected and the cancer cell is from lung tissue. In still other embodiments, annexin-1 expression levels are detected and the cancer cell is from ovarian tissue.

In yet another aspect, the invention provides a method of diagnosing chemotherapeutic drug resistance in a breast cancer cell using a plurality of at least four marker genes from the group consisting of Pgp 1, BCRP, L-plastin, annexin-1, ezrin, HnRNP, E-FABP, SOD, γ-actin, vimentin, HSC70, KAP-1, prosolin, β-tubulin, GST-Π, “similar to stratifin”, HSP90, nucleophosmin, PDI, MRP1, ATP synthase β, ATP synthase δ, tropomyosin 2β, prohibitin, 5C5-2, HSP27, HSP60, calumenin, and thioredoxine peroxidase 1. To determine drug resistance, a level of expression of the plurality of marker genes in the breast cancer cell sample is detected, and compared to the level of expression of the plurality of marker genes in a non-drug-resistant cancer cell of the same tissue type. If the level of expression of a plurality of marker genes in the breast cancer cell sample is greater than the level of expression of the same marker genes in the non-drug-resistant breast cancer cell sample, then the breast cancer cell sample is likely to be resistant to chemotherapeutic drug treatment.

In certain embodiments, the plurality of marker genes examined is at least five, and a higher level of expression of a plurality of at least three marker genes in the breast cancer cell sample compared to the non-resistant breast cancer cell is indicative of drug resistance. In other embodiments, at least six marker genes are examined, and a higher level of expression of at least four of these marker genes in the breast cancer cell sample compared to the non-resistant breast cancer cell indicates that the breast cancer cell sample is drug-resistant. In still other embodiments, the number of marker genes examined is at least seven and a higher level of expression of a plurality of at least five marker genes in the breast cancer cell sample compared to the non-resistant breast cancer cell indicates that the breast cancer cell sample is drug- resistant. In more embodiments, the number of marker genes examined is at least eight and a higher level of expression of a plurality of at least six marker genes in the breast cancer cell sample compared to the non-resistant breast cancer cell indicates that the breast cancer cell sample is drug-resistant.

In some embodiments, the level of expression of cancer cell markers is detected using capture probes that are attached to a solid support.

In still further embodiments, the number of marker genes examined is at least four and these genes are from the group consisting of prohibitin, Pgp 1, calumenin, tropomyosin 2β, L-plastin, “similar to stratifin,” and prefoldin subunit 1. In these embodiments, a higher level of expression of at least three marker genes in the breast cancer cell sample compared to the non-resistant breast cancer cell is indicative of drug resistance in the breast cancer cell sample.

In particular embodiments, a higher level of expression of annexin-1 in the breast cancer cell sample compared to the non-resistant breast cancer cell indicates that the breast cancer cell sample is drug-resistant.

In an additional aspect, the invention provides a method of diagnosing chemotherapeutic drug resistance in a lung cancer cell by examining at least four marker genes from the group consisting of Pgp 1, annexin-1, γ-actin, vimentin, galectin-1, β-tubulin, α-enolase, HSP90, nucleophosmin, MRP1, keratin type II, ATP synthase δ, tropomyosin 2β, prohibitin, calumenin, 5C5-2, and SLC9A3R1. To determine drug resistance, the level of expression of these marker genes in the lung cancer cell sample is detected, and then compared to the level of expression of the same marker genes in the non-drug-resistant cancer cell of the same tissue type. If the level of expression of two or more of these marker genes in the lung cancer cell sample is higher than the level of expression of the same marker genes in the non-drug-resistant lung cancer cell sample, then the lung cancer cell sample is resistant to chemotherapeutic drug treatment.

In certain embodiments, at least five nucleic acid capture probes are used, and a higher level of expression of at least three of these marker genes in the lung cancer cell sample compared to the non-resistant lung cancer cell indicates that the lung cancer cell sample is drug-resistant. In other embodiments, at least six marker genes are examined, and a higher level of expression of at least four of these marker genes in the lung cancer cell sample compared to the non-resistant lung cancer cell indicates that the lung cancer cell sample is drug-resistant. In more embodiments, at least seven marker genes are examined, and a higher level of expression of at least five of these marker genes in the lung cancer cell sample compared to the non-resistant lung cancer cell indicates that the lung cancer cell sample is drug-resistant. In still other embodiments, the plurality of marker genes selected is at least eight and a higher level of expression of a plurality of at least six marker genes in the lung cancer cell sample compared to the non-resistant lung cancer cell indicates that the lung cancer cell sample is drug-resistant.

In some embodiments, the level of expression of cancer cell markers is detected using capture probes attached to a solid support.

In further embodiments, the plurality of at least four marker genes is selected from the group consisting of Pgp 1, β-actin, prohibitin, calumenin, HSP90, ATP synthase δ, galectin-1 and keratin type II. In certain embodiments, a higher level of expression of at least three of these marker genes in the lung cancer cell sample compared to the non-resistant lung cancer cell indicates that the lung cancer cell sample is drug-resistant. In some embodiments, a higher level of expression of keratin type II in the lung cancer cell sample compared to the non-resistant lung cancer cell indicates that the lung cancer cell sample is drug-resistant.

In yet another aspect, the invention provides methods for diagnosing chemotherapeutic drug resistance in an ovarian cancer cell by examining four or more marker genes. The marker genes examined are from the group consisting of Pgp 1, P53, annexin-1, ezrin, KAP-1, HnRNP, E-FABP, HSP27, SOD, γ-actin, vimentin, HSC70, galectin-1, prosolin, β-tubulin, α-enolase, HSP90, HSP60, nucleophosmin, FAS, Rad23 homolog β, α-tubulin, MRP1, keratin type II, tropomyosin 2β, prohibitin, calumenin, 5C5-2, SLC9A3R1, pyrophosphatase inorganic, MB-COMT, EF2, PDI, and PDI/ER 60 precursor protein. The level of expression of these marker genes is detected in the ovarian cancer cell sample and compared to the level of expression of the plurality of marker genes in a non-drug-resistant cancer cell of the same tissue type. If the level of expression of a one or more of these marker genes in the ovarian cancer cell sample is greater than the level of expression of the same marker genes in the non-drug-resistant ovarian cancer cell sample, then the ovarian cancer cell sample is drug-resistant.

In certain embodiments, at least five nucleic acid capture probes are used, and a higher level of expression of at least three of these marker genes in the ovarian cancer cell sample compared to the non-resistant ovarian cancer cell indicates that the ovarian cancer cell sample is drug-resistant. In other embodiments, at least six marker genes are used, and a higher level of expression of at least four of these marker genes in the ovarian cancer cell sample compared to the non-resistant ovarian cancer cell indicates that the ovarian cancer cell sample is drug-resistant. In still other embodiments, at least seven marker genes are used and a higher level of expression of at least five of these marker genes in the ovarian cancer cell sample compared to the non-resistant ovarian cancer cell indicates that the ovarian cancer cell sample is drug-resistant. In certain embodiments, the level of expression of cancer cell markers is detected using capture probes attached to a solid support.

In other embodiments of this aspect of the invention, at least four different marker genes are detected and these marker genes are selected from the group consisting of Pgp 1, HSP60, prohibitin, galectin-1, nucleophosmin, calumenin, and annexin-1. In particular embodiments, a higher level of expression of at least three of these marker genes in the ovarian cancer cell sample compared to the non-resistant ovarian cancer cell indicates that the ovarian cancer cell sample is drug-resistant. In certain embodiments, a higher level of expression of annexin-I in the ovarian cancer cell sample compared to the non-resistant ovarian cancer cell indicates that the ovarian cancer cell sample is drug-resistant.

In still another aspect, the invention provides a focused microarray for diagnosis of chemotherapeutic drug resistance in breast cancer. The focused microarray contains a first set of nucleic acid capture probes for determining adriamycin resistance. The set has a plurality of nucleic acid capture probes in which each capture probe is complementary to a marker gene. The marker genes are selected from the group consisting of cytokeratin 7, HSC70, prosolin, ezrin, prohibitin, p16INK4a, MYL16, interleukine 18 precursor, prefoldin subunit 1, cathepsin β, and PDI. This aspect further contains a second set of nucleic acid capture probes for determining taxol resistance. The set uses a plurality of nucleic acid capture probes in which each capture probe is complementary to a marker gene. The marker genes are selected from the group consisting of cathepsin δ, PDI, and cathepsin β. The invention also uses a third set of nucleic acid capture probes for identifying a breast tumor. The set has a plurality of nucleic acid capture probes in which each capture probe is complementary to a marker gene from the group consisting of keratin 19, c-erb β2/HER-2, SLC9A3R1, and A-CRABP II. The invention additionally contains a fourth set of nucleic acid capture probes. The set has a plurality of nucleic acid capture probes in which each capture probe is complementary to a marker gene selected from the group consisting of HSP60, DADEH1, EF2, and EIF4B. The focused microarray comprises a solid support to which the nucleic acid capture probes are attached at predetermined positions.

In certain embodiments, at least three nucleic acid capture probes of the first set are complementary to marker genes selected from the group consisting of cytokeratin 7, HSC70, prosolin, ezrin, prohibitin, p16INK4a, MYL16, interleukine 18 precursor, and prefoldin subunit 1. In certain other embodiments, the first set contains at least four nucleic acid capture complementary to marker genes selected from the group consisting of cytokeratin 7, HSC70, prosolin, ezrin, prohibitin, p16INK4a, MYL16, interleukine 18 precursor, and prefoldin subunit 1.

In still other certain embodiments, at least three nucleic acid capture probes of the second set are complementary to marker genes selected from the group consisting of cathepsin δ, PDI, and cathepsin β. In still further embodiments, at least three nucleic acid capture probes of the third set are complementary to marker genes selected from the group consisting of keratin 19, c-erb β2/HER-2, SLC9A3R1, and A-CRABP II. In more embodiments, at least three nucleic acid capture probes of the fourth set are complementary to marker genes selected from the group consisting of HSP60, DADEH1, EF2, and EIF4B. In some embodiments, the plurality of nucleic acid capture probes of the first, second, third, and fourth sets is at least two marker genes.

In another aspect, the invention provides methods of diagnosing chemotherapeutic drug resistance in a breast cancer cell. The method comprises using a focused microarray that has a first set and a second set of nucleic acid capture probes. Each capture probe detects the expression level of a marker gene. The first set nucleic acid capture probes are complementary to a plurality of marker genes selected from the group consisting of keratin 19, c-erb β2/HER-2, SLC9A3R1, A-CRABP II, HSC70, prosolin, ezrin, prohibitin, p16INK4a, MYL16, interleukine 18 precursor, prefoldin subunit 1, HSP60, DADEH1, EF2, EIF4B, and PDI. The second set of nucleic acid capture probes are complementary to a plurality of marker genes selected from the group consisting of cathepsin δ, PDI, and cathepsin β. The methods further entail the detection of a level of expression of the first and the second set of marker genes in the breast cancer cell sample, and then comparing the level of expression of the first and second set of marker genes in the breast cancer cell sample to the level of expression of the same marker genes in a non-drug-resistant breast cancer cell. The breast cancer cell sample is drug-resistant if the level of expression of at least one marker gene of the first and second set in the breast cancer cell sample is greater than the level of expression of the same marker genes in the non-drug-resistant breast cancer cell.

In certain embodiments, the method comprises examining the expression levels of housekeeping genes in the breast cancer cell sample. Some housekeeping genes are selected from the group consisting of FABP7, DADEH1, EF2, EIF4B, and cathepsin β. The method of this embodiment then compares the levels of expression of the housekeeping genes in the breast cancer cell sample to the levels of expression of the marker genes in the breast cancer cell to normalize the signal detected on the focused microarray.

In some embodiments, the breast cancer cell is adriamycin-resistant if the level of expression of two or more of the first set of marker genes in the cancer cell sample is greater than the level of expression of the same marker gene(s) in the non-adriamycin-resistant breast cancer cell. In other embodiments, the breast cancer cell is adriamycin-resistant if the level of expression of three or more of the first set of marker genes in the cancer cell sample is greater than the level of expression of the same marker gene(s) in the non-adriamycin-resistant breast cancer cell. In still other embodiments, if the level of expression of four or more of the first set of marker genes in the cancer cell sample is greater than the level of expression of the same marker gene(s) in the non-adriamycin-resistant breast cancer cell, the breast cancer cell is adriamycin-resistant.

In yet other embodiments, an increased level of expression of at least two marker genes of the second set in the cancer cell sample when compared to the level of expression of the same marker gene(s) in the non-taxol-resistant breast cancer cell is indicative of taxol resistance in the breast cancer cell sample. In some embodiments, taxol resistance is indicated in a breast cancer cell if the level of expression of three or more of the second set of marker genes in the cancer cell sample is greater than the level of expression of the same marker gene(s) in the non-taxol-resistant breast cancer cell. In still further embodiments, the breast cancer cell is taxol-resistant if the level of expression of four or more of the second set of marker genes in the cancer cell sample is greater than the level of expression of the same marker gene(s) in the non-taxol-resistant breast cancer cell. In certain embodiments, the level of expression of cancer cell markers is detected using capture probes attached to a solid support.

In another aspect, the invention provides a focused microarray for diagnosis of chemotherapeutic drug resistance in ovarian cancer. The focused microarray comprises a first set of nucleic acid capture probes for determining taxol and cisplatinum resistance. The set comprises a plurality of nucleic acid capture probes that are complementary to marker genes selected from the group consisting of HSP60, nucleophosmin, ezrin, prohibitin, and cathepsin β. The focused microarray also has a second set of nucleic acid capture probes for identifying an ovarian tumor. This set contains a plurality of nucleic acid capture probes. Each capture probe is complementary to a marker gene selected from the group consisting of p53, A-CRABP II, KAP-1, and prefoldin subunit 1. The focused microarray further contains a third set of nucleic acid capture probes. The set is a plurality of nucleic acid capture probes. The capture probes are complementary to marker genes selected from the group consisting of FABP7, DADEH1, EF2, and EIF4B. Finally, the focused microarray is composed of a solid support to which the nucleic acid capture probes are attached at predetermined positions.

In some embodiments, at least three nucleic acid capture probes of the first set are complementary to marker genes selected from the group consisting of HSP60, nucleophosmin, ezrin, prohibitin, and cathepsin β. In other embodiments, at least four nucleic acid capture probes of the first set are complementary to marker genes selected from the group consisting of HSP60, nucleophosmin, ezrin, prohibitin, and cathepsin β.

In some other embodiments, at least three nucleic acid capture probes of the second set are complementary to marker genes selected from amongst p53, A-CRABP II, KAP-1, and prefoldin subunit 1. In still more embodiments, the number of nucleic acid capture probes of the third set is at least three of the capture probes complementary to marker genes selected from the group consisting of FABP7, DADEH1, EF2, and EIF4B.

In some embodiments, the number of capture probes of the first, second, and third sets is at least two.

In still another aspect, the invention provides a method of diagnosing chemotherapeutic taxol resistance in an ovarian cancer cell. The method comprises a focused microarray that has a plurality of nucleic acid capture probes. Each capture probe is complementary to marker gene selected from the group consisting of p53, A-CRABP II, KAP-1, HSP60, nucleophosmin, ezrin, prohibitin, and prefoldin subunit 1. The method comprises using the focused microarray to detect a level of expression of marker genes in the ovarian cancer cell sample. The level of expression of the marker genes in the ovarian cancer cell sample is then compared to the level of expression of the same marker genes in a taxol-sensitive ovarian cancer cell. Taxol resistance is indicated if the level of expression of at least one marker gene in the ovarian cancer cell sample is greater than the level of expression of the same marker genes in the taxol-sensitive ovarian cancer cell.

In some embodiments, the ovarian cancer cell is taxol-resistant if the level of expression of two or more of the plurality of marker genes in the cancer cell sample is greater than the level of expression of the same marker gene(s) in the non-taxol-resistant ovarian cancer cell. In other embodiments, the ovarian cancer cell is taxol-resistant if the level of expression of three or more of the plurality of marker genes in the cancer cell sample is greater than the level of expression of the same marker gene(s) in the non-taxol-resistant ovarian cancer cell. In still more embodiments, the ovarian cancer cell is taxol-resistant if the level of expression of four or more of the plurality of marker genes in the cancer cell sample is greater than the level of expression of the same marker gene(s) in the non-taxol-resistant ovarian cancer cell.

In particular embodiments, the method further comprises determining the expression levels of housekeeping genes in the ovarian cancer cell sample and the drug-sensitive cancer cell. The housekeeping genes can be selected from the group consisting of FABP7, DADEH1, EF2, EIF4B, and cathepsin β. The levels of expression of the housekeeping genes are compared to the levels of expression of marker genes in the ovarian cancer cell sample and the drug-sensitive cancer cell sample to normalize the signal.

In yet another aspect, the invention provides a focused microarray for diagnosis of chemotherapeutic drug resistance. The focused microarray has at least five nucleic acid capture probes, and each capture probe is complementary to a marker gene, such as Pgp 1, BCRP, P53, annexin-1, UCHL-1, ezrin, HnRNP, E-FABP, “similar to stratifin”, HSP27, SOD, γ-actin, vimentin, HSC70, galectin-1, prosolin, β-tubulin, GST-Π, α-enolase, HSP90, HSP60, nucleophosmin, PDI/ER-60 precursor, FAS, Rad23 homolog β, α-tubulin, MRP1, keratin type II, ATP synthase δ, tropomyosin 2β, prohibitin, calumenin, 5C5-2, SLC9A3R1, pyrophosphatase inorganic, DADEH1, EIF-4B, APRT, LRP/MVP, MB-COMT, EF2, PDI, BIP, and thioredoxine peroxidase 1. However, the focused microarray does not include a nucleic acid capture probe complementary to marker genes from the group consisting of Ki67, estrogen receptor α, estrogen receptor β, Bcl-2, cathepsin β, cathepsin δ, keratin 19, topoisomerase type II α, P53, and GAPDH. Additionally, the nucleic acid capture probes are attached to a solid support at predetermined positions.

In certain embodiments, at least one nucleic acid capture probe bind at least one marker gene from the group consisting of annexin-1, galectin-1, HSP27, keratin type II, MRP1, prohibitin, calumenin, and Pgp 1. In other embodiments, at least two nucleic acid capture probes are complementary to marker genes from the group consisting of annexin-1, galectin-1, HSP27, keratin type II, MRP1, prohibitin, calumenin, and Pgp 1. In still other embodiments, at least three nucleic acid capture probes are complementary to marker genes chosen from the group consisting of annexin-1, galectin-1, HSP27, keratin type II, MRP1, prohibitin, calumenin, and Pgp 1. In yet other embodiments, at least four nucleic acid capture probes are complementary to marker genes selected from the group consisting of annexin-1, galectin-1, HSP27, keratin type II, MRP1, prohibitin, calumenin, and Pgp 1. In certain other embodiments, at least five nucleic acid capture probes are complementary to marker genes from the group consisting of annexin-1, galectin-1, HSP27, keratin type II, MRP1, prohibitin, calumenin, and Pgp 1. In other embodiments, the solid support comprises glass, metal alloy, silicon, and nylon.

In another aspect, the invention provides a focused microarray for diagnosis of chemotherapeutic drug resistance in breast cancer. The focused microarray comprises a plurality of at least four nucleic acid capture probes, and each capture probe is complementary to a marker gene selected from the group consisting of Pgp 1, BCRP, L-plastin, annexin-1, ezrin, HnRNP, E-FABP, SOD, γ-actin, vimentin, HSC70, KAP-1, prosolin, β-tubulin, GST-Π, “similar to stratifin”, HSP90, nucleophosmin, PDI, MRP1, ATP synthase β, ATP synthase δ, tropomyosin 2β, prohibitin, 5C5-2, HSP27, HSP60, calumenin, and thioredoxine peroxidase 1. The focused microarray does not include a nucleic acid capture probe complementary to the cellular marker genes selected from the group consisting of Ki67, estrogen receptor α, estrogen receptor β, Bcl-2, cathepsin β, cathepsin δ, keratin 19, topoisomerase type II α, P53, and GAPDH. Also, the nucleic acid capture probes are attached to a solid support at predetermined positions.

In still another aspect, the invention provides a focused microarray for diagnosis of chemotherapeutic drug resistance in lung cancer. The microarray comprises at least four nucleic acid nucleic acid capture probes. Each capture probe is complementary to a marker gene selected from the group consisting of Pgp 1, annexin-1, γ-actin, vimentin, galectin-1, β-tubulin, α-enolase, HSP90, nucleophosmin, MRP1, keratin type II, ATP synthase δ, tropomyosin 2β, prohibitin, calumenin, 5C5-2, and SLC9A3R1. Also, the nucleic acid capture probes are attached to a solid support at predetermined positions.

In another aspect, the invention provides a focused microarray for diagnosis of chemotherapeutic drug resistance in ovarian cancer. The focused microarray comprises at least four nucleic acid capture probes. Each capture probe is complementary to a marker gene selected from the group consisting of Pgp 1, P53, annexin-1, ezrin, KAP-1, HnRNP, E-FABP, HSP27, SOD, γ-actin, vimentin, HSC70, galectin-1, prosolin, β-tubulin, α-enolase, HSP90, HSP60, nucleophosmin, FAS, Rad23 homolog β, α-tubulin, MRP1, keratin type II, tropomyosin 2β, prohibitin, calumenin, 5C5-2, SLC9A3R1, pyrophosphatase inorganic, MB-COMT, EF2, PDI, and PDI/ER 60 precursor protein. Also, the nucleic acid capture probes are attached to a solid support at predetermined positions.

In a further aspect, the invention provides methods of diagnosing chemotherapeutic drug resistance in a cancer cell sample using an antibody microarray. The microarray comprises a plurality of antibodies affixed to its surface. Each antibody binds to a cell marker selected from the group consisting of ezrin, HnRNP, UCHL-1, E-FABP, “similar to stratifin”, vimentin, galectin-1, GST-Π, α-enolase, NEM factor attachment protein γ, PDI/ER-60 precursor, Rad23 homolog β, prosolin, tropomyosin 2β, nucleophosmin and ETF3 subunit 2. The level of protein expression of these cell markers is detected in the cancer cell sample and compared to the level of protein expression of the plurality of cell markers in a non-drug-resistant cancer cell of the same tissue type. If the level of protein expression of one or more cell markers in the cancer cell sample is greater than the level of protein expression of the cell marker in the non-resistant cancer cell of the same tissue type, then the cancer cell sample is drug-resistant.

In some embodiments, at least two antibodies are affixed to the surface of the focused microarray. In particular embodiments, at least three antibodies are affixed to the surface of the focused microarray. In other embodiments, at least four antibodies are affixed to the surface of the focused microarray.

In certain embodiments, the plurality of antibodies binds to at least one cell marker selected from the group consisting of prosolin, E-FABP, vimentin, HnRNP, tropomyosin 2β, ezrin, galectin-1, α-enolase, and GST-Π. In other embodiments, the plurality of antibodies binds to at least two cell markers selected from the group consisting of prosolin, E-FABP, vimentin, HnRNP, tropomyosin 2β, ezrin, galectin-1, α-enolase, and GST-Π. In still other embodiments, the plurality of antibodies binds to at least three cell markers selected from the group consisting of prosolin, E-FABP, vimentin, HnRNP, tropomyosin 2β, ezrin, galectin-1, α-enolase, and GST-Π. In yet other embodiments, the plurality of antibodies binds to at least four of the cell markers selected from the group consisting of prosolin, E-FABP, vimentin, HnRNP, tropomyosin 2β, ezrin, galectin-1, α-enolase, and GST-Π. In particular embodiments, the plurality of antibodies binds to at least five cell markers selected from the group consisting of prosolin, E-FABP, vimentin, HnRNP, tropomyosin 2β, ezrin, galectin-1, α-enolase, and GST-Π. In still other embodiments, the antibodies affixed to the solid surface are IgG-type.

In certain embodiments, if the level of protein expression of at least two cell markers in the cancer cell is greater than the level of protein expression of the cell markers in the non-resistant cancer cell of the same tissue type, then the cancer cell is drug-resistant. In particular embodiments, if the level of protein expression of at least three cell markers in the cancer cell is greater than the level of protein expression of the cell markers in the non-resistant cancer cell of the same tissue type, then the cancer cell is drug-resistant. In other particular embodiments, if the level of protein expression of at least four cell markers in the cancer cell is greater than the level of protein expression of the cell markers in the non-resistant cancer cell of the same tissue type, then the cancer cell is drug-resistant.

In particular embodiments, if the level of protein expression of at least five cell markers in the cancer cell is greater than the level of protein expression of the cell markers in the non-resistant cancer cell of the same tissue type, then the cancer cell is drug-resistant. In other embodiments, if the level of protein expression of at least six cell markers in the cancer cell is greater than the level of protein expression of the cell markers in the non-resistant cancer cell of the same tissue type, then the cancer cell is drug-resistant.

In another aspect, the invention provides a focused antibody microarray for diagnosis of chemotherapeutic drug resistance. The focused antibody microarray comprises at least three antibodies that bind to cell markers selected from the group consisting of ezrin, HnRNP, UCHL-1, E-FABP, “similar to stratifin”, vimentin, galectin-1, GST-Π, α-enolase, NEM factor attachment protein γ, E-FABP, PDI/ER-60 precursor, Rad23 homolog β, prosolin, tropomyosin 2β, nucleophosmin and ETF3 subunit 2. Furthermore, the antibodies are attached to a solid support at predetermined positions.

In certain embodiments, at least four antibodies are attached to the focused microarray and each antibody binds to a cell marker. In other embodiments, the plurality of antibodies bind to at least one cell marker selected from the group consisting of prosolin, E-FABP, vimentin, HnRNP, tropomyosin 2β, ezrin, galectin-1, α-enolase, and GST-Π. In still other embodiments, a plurality antibodies bind to at least two cell markers selected from the group consisting of prosolin, E-FABP, vimentin, HnRNP, tropomyosin 2β, ezrin, galectin-1, α-enolase, and GST-Π. In yet other embodiments, the plurality of antibodies binds to at least three cell markers selected from the group consisting of prosolin, E-FABP, vimentin, HnRNP, tropomyosin 2β, ezrin, galectin-1, α-enolase, and GST-Π. In more embodiments, at least four cell markers selected from the group consisting of prosolin, E-FABP, vimentin, HnRNP, tropomyosin 2β, ezrin, galectin-1, α-enolase, and GST-Π are bound by a plurality of antibodies. In still more particular embodiments, the plurality of antibodies binds to at least five cell markers such as prosolin, E-FABP, vimentin, HnRNP, tropomyosin 2β, ezrin, galectin-1, α-enolase, and GST-Π.

In certain embodiments, the antibodies affixed to the solid surface are IgG-type. In particular embodiments, the solid support is composed of glass, metal alloy, silicon, or nylon.

In still another aspect, the invention provides methods of diagnosing chemotherapeutic drug resistance in a cancer cell sample. The methods involves using a plurality of cell markers selected from the group consisting of ezrin, HnRNP, UCHL-1, E-FABP, “similar to stratifin”, vimentin, galectin-1, GST-Π, α-enolase, NEM factor attachment protein γ, PDI/ER-60 precursor, Rad23 homolog β, prosolin, tropomyosin 2β, nucleophosmin and ETF3 subunit 2. The level of protein expression of these cell markers is detected in the cancer cell sample, and compared to the level of expression of the same cell markers in a non-drug-resistant cancer cell of the same tissue type. If the level of protein expression of one or more of these cell markers in the cancer cell sample is greater than the level of protein expression of the same cell markers in the non-resistant cancer cell of the same tissue type, then the cancer cell sample is drug-resistant.

In certain embodiments, at least three cell markers are detected. If the level of protein expression of at least two of these three cell markers in the cancer cell sample is greater than the level of protein expression of the same cell markers in the non-resistant cancer cell of the same tissue type, then the cancer cell is drug-resistant. In other embodiments, at least four cell markers are detected. If the level of protein expression of at least three of these four cell markers in the cancer cell sample is greater than the level of protein expression of the same cell markers in the non-resistant cancer cell of the same tissue type, then the cancer cell is drug-resistant. In still other embodiments, at least five cell markers are detected. Detection of increased expression of at least four of these five cell markers in the cancer cell sample as compared to the non-resistant cancer cell of the same tissue type is indicative of drug resistance. In useful embodiments, an antibody detects the level of expression of a cell marker.

In certain embodiments, the cancer cell sample is a breast cancer sample. In particular embodiments, if the level of protein expression of at least two cell markers in the breast cancer cell sample is greater than the level of protein expression of the cell markers in a non-resistant breast cancer cell, then the breast cancer cell sample is drug-resistant. In other embodiments, detection of increased expression of at least three cell markers in the breast cancer cell sample as compared to the level of protein expression of the same cell markers in a non-resistant breast cancer cell indicates that the cancer cell is drug-resistant. In still other embodiments, detection of increased expression of at least four cell markers in the breast cancer cell sample as compared to the levels of protein expression of the same cell markers in a non-resistant breast cancer cell is indicative of drug resistance.

### BRIEF DESCRIPTION OF THE FIGURES

The foregoing and other objects of the present invention, the various features thereof, as well as the invention itself may be more fully understood from the following description, when read together with the accompanying drawings in which:

FIG. 1A is a photographic representation of a hybridization control hybridized with cell samples obtained from MDA cell lines sensitive to mitoxantrone and MDA cell lines resistant to mitoxantrone to validate three independent hybridizations on the same microarray.

FIG. 1B is a photographic representation of a hybridization control hybridized with pre-hybridization buffer to validate three independent hybridizations on the same microarray.

FIG. 1C is a photographic representation of a hybridization control hybridized with cell samples obtained from MDA cell lines sensitive to mitoxantrone and MDA cell lines resistant to mitoxantrone to validate three independent hybridizations on the same microarray.

FIG. 2 is a graphic representation showing the results of a microarray analysis comparing the levels of expression of bcrp mRNA in different drug-resistant cell lines (e.g., MCF-7, MDA, SKOV3, 2008, T84, HCT-116, H69, H460, A549, OVCAR3, and PC3), which were resistant to varying concentrations of chemotherapeutic drugs (e.g., AR 4.8 μM, VCR 10 nM, Mito 80 nM, Taxol 160 nM, Cisp 5 μM, Mel 1 μM, etc.), to the levels of expression of bcrp mRNA in non-resistant cell lines of the same tissue type.

FIG. 3 is a graphic representation showing the results of a microarray analysis comparing the levels of expression of mrp1 mRNA in different drug-resistant cell lines (e.g., MCF-7, MDA, SKOV3, 2008, T84, HCT-116, H69, H460, A549, OVCAR3, and PC3), which were resistant to varying concentrations of chemotherapeutic drugs (e.g., AR 4.8 μM, VCR 10 nM, Mito 80 nM, Taxol 160 nM, Cisp 5 μM, Mel 1 μM, etc.), to the levels of expression of mrp1 mRNA in non-resistant cell lines of the same tissue type.

FIG. 4 is a graphic representation showing the results of a microarray analysis comparing the levels of expression of Pgp 1 mRNA in different drug-resistant cell lines (e.g., MCF-7, MDA, SKOV3, 2008, T84, HCT-116, H69, H460, A549, OVCAR3, and PC3), which were resistant to varying concentrations of chemotherapeutic drugs (e.g., AR 4.8 μM, VCR 10 nM, Mito 80 nM, Taxol 160 nM, Cisp 5 μM, Mel 1 μM, etc.), to the levels of expression of Pgp 1 mRNA in non-resistant cell lines of the same tissue type.

FIG. 5 is a graphic representation showing the results of a microarray analysis comparing the levels of expression of fabp7 mRNA in different drug-resistant cell lines (e.g., MCF-7, MDA, SKOV3, 2008, T84, HCT-116, H69, H460, A549, OVCAR3, and PC3), which were resistant to varying concentrations of chemotherapeutic drugs (e.g., AR 4.8 μM, VCR 10 nM, Mito 80 nM, Taxol 160 nM, Cisp 5 μM, Mel 1 μM, etc.), to the levels of expression of fabp7 mRNA in non-resistant cell lines of the same tissue type.

FIG. 6 is a graphic representation showing the results of a microarray analysis comparing the levels of expression of lrp/mvp mRNA in different drug-resistant cell lines (e.g., MCF-7, MDA, SKOV3, 2008, T84, HCT-116, H69, H460, A549, OVCAR3, and PC3), which were resistant to varying concentrations of chemotherapeutic drugs (e.g., AR 4.8 μM, VCR 10 nM, Mito 80 nM, Taxol 160 nM, Cisp 5 μM, Mel 1 μM, etc.), to the levels of expression of lrp/mvp mRNA in non-resistant cell lines of the same tissue type.

FIG. 7 is a graphic representation showing the results of a microarray analysis comparing the levels of expression of hsp90 mRNA in different drug-resistant cell lines (e.g., MCF-7, MDA, SKOV3, 2008, T84, HCT-116, H69, H460, A549, OVCAR3, and PC3), which were resistant to varying concentrations of chemotherapeutic drugs (e.g., AR 4.8 μM, VCR 10 nM, Mito 80 nM, Taxol 160 nM, Cisp 5 μM, Mel 1 μM, etc.), to the levels of expression of hsp90 mRNA in non-resistant cell lines of the same tissue type.

FIG. 8 is a graphic representation showing the results of a microarray analysis comparing the levels of expression of hsp60 mRNA in different drug-resistant cell lines (e.g., MCF-7, MDA, SKOV3, 2008, T84, HCT-116, H69, H460, A549, OVCAR3, and PC3), which were resistant to varying concentrations of chemotherapeutic drugs (e.g., AR 4.8 μM, VCR 10 nM, Mito 80 nM, Taxol 160 nM, Cisp 5 μM, Mel 1 μM, etc.), to the levels of expression of hsp60 mRNA in non-resistant cell lines of the same tissue type.

FIG. 9 is a graphic representation showing the results of a microarray analysis comparing the levels of expression of γ-actin mRNA in different drug-resistant cell lines (e.g., MCF-7, MDA, SKOV3, 2008, T84, HCT-116, H69, H460, A549, OVCAR3, and PC3), which were resistant to varying concentrations of chemotherapeutic drugs (e.g., AR 4.8 μM, VCR 10 nM, Mito 80 nM, Taxol 160 nM, Cisp 5 μM, Mel 1 μM, etc.), to the levels of expression of γ-actin mRNA in non-resistant cell lines of the same tissue type.

FIG. 10A is a photographic representation of a 2-D gel of Gelcode Blue stained MCF7 cell extracts that shows the level of expression of vimentin protein.

FIG. 10B is a photographic representation of a 2-D gel of Gelcode Blue stained MCF7 adriamycin-resistant cell extracts that shows the level of expression of vimentin protein.

FIG. 10C is a graphic representation showing the results of a microarray analysis comparing the levels of expression of vimentin mRNA in different drug-resistant cell lines (e.g., MCF-7, MDA, SKOV3, 2008, T84, HCT-116, H69, H460, A549, OVCAR3, and PC3), which were resistant to varying concentrations of chemotherapeutic drugs (e.g., AR 4.8 μM, VCR 10 nM, Mito 80 nM, Taxol 160 nM, Cisp 5 μM, Mel 1 μM, etc.), to the levels of expression of vimentin mRNA in non-resistant cell lines of the same tissue type.

FIG. 11 is a graphic representation showing the results of a microarray analysis comparing the levels of expression of bip mRNA in different drug-resistant cell lines (e.g., MCF-7, MDA, SKOV3, 2008, T84, HCT-116, H69, H460, A549, OVCAR3, and PC3), which were resistant to varying concentrations of chemotherapeutic drugs (e.g., AR 4.8 μM, VCR 10 nM, Mito 80 nM, Taxol 160 nM, Cisp 5 μM, Mel 1 μM, etc.), to the levels of expression of bip mRNA in non-resistant cell lines of the same tissue type.

FIG. 12 is a graphic representation showing the results of a microarray analysis comparing the levels of expression of annexin-1 mRNA in different drug-resistant cell lines (e.g., MCF-7, MDA, SKOV3, 2008, T84, HCT-116, H69, H460, A549, OVCAR3, and PC3), which were resistant to varying concentrations of chemotherapeutic drugs (e.g., AR 4.8 μM, VCR 10 nM, Mito 80 nM, Taxol 160 nM, Cisp 5 μM, Mel 1 μM, etc.), to the levels of expression of annexin-1 mRNA in non-resistant cell lines of the same tissue type.

FIG. 13A is a photographic representation of a 2-D gel of Gelcode Blue stained CEM cell extracts that shows the level of expression of nucleophosmin protein.

FIG. 13B is a photographic representation of a 2-D gel of Gelcode Blue stained CEM vinblastin-resistant cell extracts that shows the level of expression of nucleophosmin protein.

FIG. 13C is a graphic representation showing the results of a microarray analysis comparing the levels of expression of nucleophosmin mRNA in different drug-resistant cell lines (e.g., MCF-7, MDA, SKOV3, 2008, T84, HCT-116, H69, H460, A549, OVCAR3, and PC3), which were resistant to varying concentrations of chemotherapeutic drugs (e.g., AR 4.8 μM, VCR 10 nM, Mito 80 nM, Taxol 160 nM, Cisp 5 μM, Mel 1 μM, etc.), to the levels of expression of nucleophosmin mRNA in non-resistant cell lines of the same tissue type.

FIG. 14 is a graphic representation showing the results of a microarray analysis comparing the levels of expression of hsc70 mRNA in different drug-resistant cell lines (e.g., MCF-7, MDA, SKOV3, 2008, T84, HCT-116, H69, H460, A549, OVCAR3, and PC3), which were resistant to varying concentrations of chemotherapeutic drugs (e.g., AR 4.8 μM, VCR 10 nM, Mito 80 nM, Taxol 160 nM, Cisp 5 μM, Mel 1 μM, etc.), to the levels of expression of hsc70 mRNA in non-resistant cell lines of the same tissue type.

FIG. 15A is a photographic representation of a 2-D gel of Gelcode Blue stained MCF7 cell extracts that shows the level of expression of galectin 1 protein.

FIG. 15B is a photographic representation of a 2-D gel of Gelcode Blue stained MCF7 adriamycin-resistant cell extracts that shows the level of expression of galectin 1 protein.

FIG. 15C is a graphic representation showing the results of a microarray analysis comparing the levels of expression of galectin 1 mRNA in different drug-resistant cell lines (e.g., MCF-7, MDA, SKOV3, 2008, T84, HCT-116, H69, H460, A549, OVCAR3, and PC3), which were resistant to varying concentrations of chemotherapeutic drugs (e.g., AR 4.8 μM, VCR 10 nM, Mito 80 nM, Taxol 160 nM, Cisp 5 μM, Mel 1 μM, etc.), to the levels of expression of galectin 1 mRNA in non-resistant cell lines of the same tissue type.

FIG. 16 is a graphic representation showing the results of a microarray analysis comparing the levels of expression of hsp27 mRNA in different drug-resistant cell lines (e.g., MCF-7, MDA, SKOV3, 2008, T84, HCT-116, H69, H460, A549, OVCAR3, and PC3), which were resistant to varying concentrations of chemotherapeutic drugs (e.g., AR 4.8 μM, VCR 10 nM, Mito 80 nM, Taxol 160 nM, Cisp 5 μM, Mel 1 μM, etc.), to the levels of expression of hsp27 mRNA in non-resistant cell lines of the same tissue type.

FIG. 17A is a photographic representation of a 2-D gel of Gelcode Blue stained MCF7 cell extracts that shows the level of expression of UCHL-1 protein.

FIG. 17B is a photographic representation of a 2-D gel of Gelcode Blue stained MCF7 adriamycin-resistant cell extracts that shows the level of expression of UCHL-1 protein.

FIG. 17C is a graphic representation showing the results of a microarray analysis comparing the levels of expression of uchl-1 mRNA in different drug-resistant cell lines (e.g., MCF-7, MDA, SKOV3, 2008, T84, HCT-116, H69, H460, A549, OVCAR3, and PC3), which were resistant to varying concentrations of chemotherapeutic drugs (e.g., AR 4.8 μM, VCR 10 nM, Mito 80 nM, Taxol 160 nM, Cisp 5 μM, Mel 1 μM, etc.), to the levels of expression of uchl-1 mRNA in non-resistant cell lines of the same tissue type.

FIG. 18 is a graphic representation showing the results of a microarray analysis comparing the levels of expression of atp synthase β mRNA in different drug-resistant cell lines (e.g., MCF-7, MDA, SKOV3, 2008, T84, HCT-116, H69, H460, A549, OVCAR3, and PC3), which were resistant to varying concentrations of chemotherapeutic drugs (e.g., AR 4.8 μM, VCR 10 nM, Mito 80 nM, Taxol 160 nM, Cisp 5 μM, Mel 1 μM, etc.), to the levels of expression of atp synthase β mRNA in non-resistant cell lines of the same tissue type.

FIG. 19A is a photographic representation of a 2-D gel of Gelcode Blue stained MCF7 cell extracts that shows the level of expression of prosolin protein.

FIG. 19B is a photographic representation of a 2-D gel of Gelcode Blue stained MCF7 adriamycin-resistant cell extracts that shows the level of expression of prosolin protein.

FIG. 19C is a graphic representation showing the results of a microarray analysis comparing the levels of expression of prosolin mRNA in different drug-resistant cell lines (e.g., MCF-7, MDA, SKOV3, 2008, T84, HCT-116, H69, H460, A549, OVCAR3, and PC3), which were resistant to varying concentrations of chemotherapeutic drugs (e.g., AR 4.8 μM, VCR 10 nM, Mito 80 nM, Taxol 160 nM, Cisp 5 μM, Mel 1 μM, etc.), to the levels of expression of prosolin mRNA in non-resistant cell lines of the same tissue type.

FIG. 20 is a graphic representation showing the results of a microarray analysis comparing the levels of expression of thioredoxine peroxidase mRNA in different drug-resistant cell lines (e.g., MCF-7, MDA, SKOV3, 2008, T84, HCT-116, H69, H460, A549, OVCAR3, and PC3), which were resistant to varying concentrations of chemotherapeutic drugs (e.g., AR 4.8 μM, VCR 10 nM, Mito 80 nM, Taxol 160 nM, Cisp 5 ρM, Mel 1 μM, etc.), to the levels of expression of thioredoxine peroxidase mRNA in non-resistant cell lines of the same tissue type.

FIG. 21 is a graphic representation showing the results of a microarray analysis comparing the levels of expression of β-tubulin mRNA in different drug-resistant cell lines (e.g., MCF-7, MDA, SKOV3, 2008, T84, HCT-116, H69, H460, A549, OVCAR3, and PC3), which were resistant to varying concentrations of chemotherapeutic drugs (e.g., AR 4.8 μM, VCR 10 nM, Mito 80 nM, Taxol 160 nM, Cisp 5 μM, Mel 1 μM, etc.), to the levels of expression off β-tubulin mRNA in non-resistant cell lines of the same tissue type.

FIG. 22A is a photographic representation of a 2-D gel of Gelcode Blue stained MCF7 cell extracts that shows the level of expression of ezrin protein.

FIG. 22B is a photographic representation of a 2-D gel of Gelcode Blue stained MCF7 adriamycin-resistant cell extracts that shows the level of expression of ezrin protein.

FIG. 22C is a graphic representation showing the results of a microarray analysis comparing the levels of expression of ezrin mRNA in different drug-resistant cell lines (e.g., MCF-7, MDA, SKOV3, 2008, T84, HCT-116, H69, H460, A549, OVCAR3, and PC3), which were resistant to varying concentrations of chemotherapeutic drugs (e.g., AR 4.8 μM, VCR 10 nM, Mito 80 nM, Taxol 160 nM, Cisp 5 μM, Mel 1 μM, etc.), to the levels of expression of ezrin mRNA in non-resistant cell lines of the same tissue type.

FIG. 23 is a graphic representation showing the results of a microarray analysis comparing the levels of expression of kap1 mRNA in different drug-resistant cell lines (e.g., MCF-7, MDA, SKOV3, 2008, T84, HCT-116, H69, H460, A549, OVCAR3, and PC3), which were resistant to varying concentrations of chemotherapeutic drugs (e.g., AR 4.8 μM, VCR 10 nM, Mito 80 nM, Taxol 160 nM, Cisp 5 μM, Mel 1 μM, etc.), to the levels of expression of kap1 mRNA in non-resistant cell lines of the same tissue type.

FIG. 24 is a graphic representation showing the results of a microarray analysis comparing the levels of expression of phosphatase inorganic mRNA in different drug-resistant cell lines (e.g., MCF-7, MDA, SKOV3, 2008, T84, HCT-116, H69, H460, A549, OVCAR3, and PC3), which were resistant to varying concentrations of chemotherapeutic drugs (e.g., AR 4.8 μM, VCR 10 nM, Mito 80 nM, Taxol 160 nM, Cisp 5 μM, Mel 1 μM, etc.), to the levels of expression of phosphatase inorganic mRNA in non-resistant cell lines of the same tissue type.

FIG. 25A is a photographic representation of a 2-D gel of Gelcode Blue stained MCF7 cell extracts that shows the level of expression of GST-σ protein.

FIG. 25B is a photographic representation of a 2-D gel of Gelcode Blue stained MCF7 adriamycin-resistant cell extracts that shows the level of expression of GST-σ protein.

FIG. 25C is a graphic representation showing the results of a microarray analysis comparing the levels of expression of GST-σ mRNA in different drug-resistant cell lines (e.g., MCF-7, MDA, SKOV3, 2008, T84, HCT-116, H69, H460, A549, OVCAR3, and PC3), which were resistant to varying concentrations of chemotherapeutic drugs (e.g., AR 4.8 μM, VCR 10 nM, Mito 80 nM, Taxol 160 nM, Cisp 5 μM, Mel 1 μM, etc.), to the levels of expression of GST-σ mRNA in non-resistant cell lines of the same tissue type.

FIG. 26 is a graphic representation showing the results of a microarray analysis comparing the levels of expression of atp synthase δ mRNA in different drug-resistant cell lines (e.g., MCF-7, MDA, SKOV3, 2008, T84, HCT-116, H69, H460, A549, OVCAR3, and PC3), which were resistant to varying concentrations of chemotherapeutic drugs (e.g., AR 4.8 μM, VCR 10 nM, Mito 80 nM, Taxol 160 nM, Cisp 5 μM, Mel 1 μM, etc.), to the levels of expression of atp synthase δ mRNA in non-resistant cell lines of the same tissue type.

FIG. 27 is a graphic representation showing the results of a microarray analysis comparing the levels of expression of protein disulfide isomerase precursor mRNA in different drug-resistant cell lines (e.g., MCF-7, MDA, SKOV3, 2008, T84, HCT-116, H69, H460, A549, OVCAR3, and PC3), which were resistant to varying concentrations of chemotherapeutic drugs (e.g., AR 4.8 μM, VCR 10 nM, Mito 80 nM, Taxol 160 nM, Cisp 5 μM, Mel 1 μM, etc.), to the levels of expression of protein disulfide isomerase precursor mRNA in non-resistant cell lines of the same tissue type.

FIG. 28A is a photographic representation of a 2-D gel of Gelcode Blue stained MCF7 cell extracts that shows the level of expression of DADEH 1 protein.

FIG. 28B is a photographic representation of a 2-D gel of Gelcode Blue stained MCF7 adriamycin-resistant cell extracts that shows the level of expression of DADEH1 protein.

FIG. 28C is a graphic representation showing the results of a microarray analysis comparing the levels of expression of dadeh1 mRNA in different drug-resistant cell lines (e.g., MCF-7, MDA, SKOV3, 2008, T84, HCT-116, H69, H460, A549, OVCAR3, and PC3), which were resistant to varying concentrations of chemotherapeutic drugs (e.g., AR 4.8 μM, VCR 10 nM, Mito 80 nM, Taxol 160 nM, Cisp 5 μM, Mel 1 μM, etc.), to the levels of expression of dadeh1 mRNA in non-resistant cell lines of the same tissue type.

FIG. 29 is a graphic representation showing the results of a microarray analysis comparing the levels of expression of ef-2 mRNA in different drug-resistant cell lines (e.g., MCF-7, MDA, SKOV3, 2008, T84, HCT-116, H69, H460, A549, OVCAR3, and PC3), which were resistant to varying concentrations of chemotherapeutic drugs (e.g., AR 4.8 μM, VCR 10 nM, Mito 80 nM, Taxol 160 nM, Cisp 5 μM, Mel 1 μM, etc.), to the levels of expression of ef-2 mRNA in non-resistant cell lines of the same tissue type.

FIG. 30A is a photographic representation of a 2-D gel of Gelcode Blue stained MCF7 cell extracts that shows the level of expression of a-enolase protein.

FIG. 30B is a photographic representation of a 2-D gel of Gelcode Blue stained MCF7 adriamycin-resistant cell extracts that shows the level of expression of α-enolase protein.

FIG. 30C is a graphic representation showing the results of a microarray analysis comparing the levels of expression of α-enolase mRNA in different drug-resistant cell lines (e.g., MCF-7, MDA, SKOV3, 2008, T84, HCT-116, H69, H460, A549, OVCAR3, and PC3), which were resistant to varying concentrations of chemotherapeutic drugs (e.g., AR 4.8 μM, VCR 10 nM, Mito 80 nM, Taxol 160 nM, Cisp 5 μM, Mel 1 μM, etc.), to the levels of expression of α-enolase mRNA in non-resistant cell lines of the same tissue type.

FIG. 31A is a photographic representation of a 2-D gel of Gelcode Blue stained MCF7 cell extracts that shows the level of expression of ETF3 subunit 2 protein.

FIG. 31B is a photographic representation of a 2-D gel of Gelcode Blue stained MCF7 adriamycin-resistant cell extracts that shows the level of expression of ETF3 subunit 2 protein.

FIG. 32A is a photographic representation of a 2-D gel of Gelcode Blue stained MCF7 cell extracts that shows the level of expression of HnRNP F protein.

FIG. 32B is a photographic representation of a 2-D gel of Gelcode Blue stained MCF7 adriamycin-resistant cell extracts that shows the level of expression of HnRNP F protein.

FIG. 32C is a graphic representation showing the results of a microarray analysis comparing the levels of expression of hnrnp F mRNA in different drug-resistant cell lines (e.g., MCF-7, MDA, SKOV3, 2008, T84, HCT-116, H69, H460, A549, OVCAR3, and PC3), which were resistant to varying concentrations of chemotherapeutic drugs (e.g., AR 4.8 μM, VCR 10 nM, Mito 80 nM, Taxol 160 nM, Cisp 5 μM, Mel 1 μM, etc.), to the levels of expression of hnrnp F mRNA in non-resistant cell lines of the same tissue type.

FIG. 33A is a photographic representation of a 2-D gel of Gelcode Blue stained MCF7 cell extracts that shows the level of expression of tropomyosin 2β protein.

FIG. 33B is a photographic representation of a 2-D gel of Gelcode Blue stained MCF7 adriamycin-resistant cell extracts that shows the level of expression of tropomyosin 2β protein.

FIG. 33C is a graphic representation showing the results of a microarray analysis comparing the levels of expression of tropomyosin 2β mRNA in different drug-resistant cell lines (e.g., MCF-7, MDA, SKOV3, 2008, T84, HCT-116, H69, H460, A549, OVCAR3, and PC3), which were resistant to varying concentrations of chemotherapeutic drugs (e.g., AR 4.8 μM, VCR 10 nM, Mito 80 nM, Taxol 160 nM, Cisp 5 μM, Mel 1 μM, etc.), to the levels of expression of tropomyosin 2β mRNA in non-resistant cell lines of the same tissue type.

FIG. 34 is a graphic representation showing the results of a microarray analysis comparing the levels of expression of eif 4β mRNA in different drug-resistant cell lines (e.g., MCF-7, MDA, SKOV3, 2008, T84, HCT-116, H69, H460, A549, OVCAR3, and PC3), which were resistant to varying concentrations of chemotherapeutic drugs (e.g., AR 4.8 μM, VCR 10 nM, Mito 80 nM, Taxol 160 nM, Cisp 5 μM, Mel 1 μM, etc.), to the levels of expression of eif 4B mRNA in non-resistant cell lines of the same tissue type.

FIG. 35 is a graphic representation showing the results of a microarray analysis comparing the levels of expression of keratin type II mRNA in different drug-resistant cell lines (e.g., MCF-7, MDA, SKOV3, 2008, T84, HCT-116, H69, H460, A549, OVCAR3, and PC3), which were resistant to varying concentrations of chemotherapeutic drugs (e.g., AR 4.8 μM, VCR 10 nM, Mito 80 nM, Taxol 160 nM, Cisp 5 μM, Mel 1 μM, etc.), to the levels of expression of keratin type II mRNA in non-resistant cell lines of the same tissue type.

FIG. 36 is a graphic representation showing the results of a microarray analysis comparing the levels of expression of prohibitin mRNA in different drug-resistant cell lines (e.g., MCF-7, MDA, SKOV3, 2008, T84, HCT-116, H69, H460, A549, OVCAR3, and PC3), which were resistant to varying concentrations of chemotherapeutic drugs (e.g., AR 4.8 μM, VCR 10 nM, Mito 80 nM, Taxol 160 nM, Cisp 5 μM, Mel 1 μM, etc.), to the levels of expression of prohibitin mRNA in non-resistant cell lines of the same tissue type.

FIG. 37 is a graphic representation showing the results of a microarray analysis comparing the levels of expression of slc9a3r1 mRNA in different drug-resistant cell lines (e.g., MCF-7, MDA, SKOV3, 2008, T84, HCT-116, H69, H460, A549, OVCAR3, and PC3), which were resistant to varying concentrations of chemotherapeutic drugs (e.g., AR 4.8 μM, VCR 10 nM, Mito 80 nM, Taxol 160 nM, Cisp 5 μM, Mel 1 μM, etc.), to the levels of expression of slc9a3r1 mRNA in non-resistant cell lines of the same tissue type.

FIG. 38 is a graphic representation showing the results of a microarray analysis comparing the levels of expression of 5c5-2 mRNA in different drug-resistant cell lines (e.g., MCF-7, MDA, SKOV3, 2008, T84, HCT-116, H69, H460, A549, OVCAR3, and PC3), which were resistant to varying concentrations of chemotherapeutic drugs (e.g., AR 4.8 μM, VCR 10 nM, Mito 80 nM, Taxol 160 nM, Cisp 5 μM, Mel 1 μM, etc.), to the levels of expression of 5c5-2 mRNA in non-resistant cell lines of the same tissue type.

FIG. 39A is a photographic representation of a 2-D gel of Gelcode Blue stained MCF7 cell extracts that shows the level of expression of PDI-ER60 protein.

FIG. 39B is a photographic representation of a 2-D gel of Gelcode Blue stained MCF7 adriamycin-resistant cell extracts that shows the level of expression of PDI-ER60 protein.

FIG. 39C is a graphic representation showing the results of a microarray analysis comparing the levels of expression of pdi-er60 mRNA in different drug-resistant cell lines (e.g., MCF-7, MDA, SKOV3, 2008, T84, HCT-116, H69, H460, A549, OVCAR3, and PC3), which were resistant to varying concentrations of chemotherapeutic drugs (e.g., AR 4.8 μM, VCR 10 nM, Mito 80 nM, Taxol 160 nM, Cisp 5 μM, Mel 1 μM, etc.), to the levels of expression of pdi-er60 mRNA in non-resistant cell lines of the same tissue type.

FIG. 40 is a graphic representation showing the results of a microarray analysis comparing the levels of expression of sod mRNA in different drug-resistant cell lines (e.g., MCF-7, MDA, SKOV3, 2008, T84, HCT-116, H69, H460, A549, OVCAR3, and PC3), which were resistant to varying concentrations of chemotherapeutic drugs (e.g., AR 4.8 μM, VCR 10 nM, Mito 80 nM, Taxol 160 nM, Cisp 5 μM, Mel 1 μM, etc.), to the levels of expression of sod mRNA in non-resistant cell lines of the same tissue type.

FIG. 41A is a photographic representation of a 2-D gel of Gelcode Blue stained CEM cell extracts that shows the level of expression of caspase recruitment domain protein 14.

FIG. 41B is a photographic representation of a 2-D gel of Gelcode Blue stained CEM vinblastin-resistant cell extracts that shows the level of expression of caspase recruitment domain protein 14.

FIG. 42A is a photographic representation of a 2-D gel of Gelcode Blue stained CEM cell extracts that shows the level of expression of NEM-sensitive factor attachment protein γ.

FIG. 42B is a photographic representation of a 2-D gel of Gelcode Blue stained CEM vinblastin-resistant cell extracts that shows the level of expression of NEM-sensitive factor attachment protein γ.

FIG. 43 is a graphic representation showing the results of a microarray analysis comparing the levels of expression of fas mRNA in different drug-resistant cell lines (e.g., MCF-7, MDA, SKOV3, 2008, T84, HCT-116, H69, H460, A549, OVCAR3, and PC3), which were resistant to varying concentrations of chemotherapeutic drugs (e.g., AR 4.8 μM, VCR 10 nM, Mito 80 nM, Taxol 160 nM, Cisp 5 μM, Mel 1 μM, etc.), to the levels of expression of fas mRNA in non-resistant cell lines of the same tissue type.

FIG. 44A is a photographic representation of a 2-D gel of Gelcode Blue stained CEM cell extracts that shows the level of expression of rad23 homologue β.

FIG. 44B is a photographic representation of a 2-D gel of Gelcode Blue stained CEM vinblastin-resistant cell extracts that shows the level of expression of rad23 homologue β.

FIG. 45 is a graphic representation showing the results of a microarray analysis comparing the levels of expression of α-tubulin mRNA in different drug-resistant cell lines (e.g., MCF-7, MDA, SKOV3, 2008, T84, HCT-116, H69, H460, A549, OVCAR3, and PC3), which were resistant to varying concentrations of chemotherapeutic drugs (e.g., AR 4.8 μM, VCR 10 nM, Mito 80 nM, Taxol 160 nM, Cisp 5 μM, Mel 1 μM, etc.), to the levels of expression of α-tubulin mRNA in non-resistant cell lines of the same tissue type.

FIG. 46A is a photographic representation of a 2-D gel of Gelcode Blue stained MCF7 cell extracts that shows the level of expression of E-FABP protein.

FIG. 46B is a photographic representation of a 2-D gel of Gelcode Blue stained MCF7 adriamycin-resistant cell extracts that shows the level of expression of E-FABP protein.

FIG. 46C is a graphic representation showing the results of a microarray analysis comparing the levels of expression of e-fabp mRNA in different drug-resistant cell lines (e.g., MCF-7, MDA, SKOV3, 2008, T84, HCT-116, H69, H460, A549, OVCAR3, and PC3), which were resistant to varying concentrations of chemotherapeutic drugs (e.g., AR 4.8 μM, VCR 10 nM, Mito 80 nM, Taxol 160 nM, Cisp 5 μM, Mel 1 μM, etc.), to the levels of expression of e-fabp mRNA in non-resistant cell lines of the same tissue type.

FIG. 47A is a photographic representation of a 2-D gel of Gelcode Blue stained MCF7 cell extracts that shows the level of expression of “similar to stratifin” protein.

FIG. 47B is a photographic representation of a 2-D gel of Gelcode Blue stained MCF7 adriamycin-resistant cell extracts that shows the level of expression of “similar to stratifin” protein.

FIG. 47C is a graphic representation showing the results of a microarray analysis comparing the levels of expression of similar to stratifin mRNA in different drug-resistant cell lines (e.g., MCF-7, MDA, SKOV3, 2008, T84, HCT-116, H69, H460, A549, OVCAR3, and PC3), which were resistant to varying concentrations of chemotherapeutic drugs (e.g., AR 4.8 μM, VCR 10 nM, Mito 80 nM, Taxol 160 nM, Cisp 5 μM, Mel 1 μM, etc.), to the levels of expression of similar to stratifin mRNA in non-resistant cell lines of the same tissue type.

FIG. 48 is a graphic representation showing the results of a microarray analysis comparing the levels of expression of p16 ink4a mRNA in different drug-resistant cell lines (e.g., MCF-7, MDA, SKOV3, 2008, T84, HCT-116, H69, H460, A549, OVCAR3, and PC3), which were resistant to varying concentrations of chemotherapeutic drugs (e.g., AR 4.8 μM, VCR 10 nM, Mito 80 nM, Taxol 160 nM, Cisp 5 μM, Mel 1 μM, etc.), to the levels of expression of p16 ink4a mRNA in non-resistant cell lines of the same tissue type.

FIG. 49 is a graphic representation showing the results of a microarray analysis comparing the levels of expression of aprt mRNA in different drug-resistant cell lines (e.g., MCF-7, MDA, SKOV3, 2008, T84, HCT-116, H69, H460, A549, OVCAR3, and PC3), which were resistant to varying concentrations of chemotherapeutic drugs (e.g., AR 4.8 μM, VCR 10 nM, Mito 80 nM, Taxol 160 nM, Cisp 5 μM, Mel 1 μM, etc.), to the levels of expression of aprt mRNA in non-resistant cell lines of the same tissue type.

FIG. 50 is a graphic representation showing the results of a microarray analysis comparing the levels of expression of calumenin mRNA in different drug-resistant cell lines (e.g., MCF-7, MDA, SKOV3, 2008, T84, HCT-116, H69, H460, A549, OVCAR3, and PC3), which were resistant to varying concentrations of chemotherapeutic drugs (e.g., AR 4.8 μM, VCR 10 nM, Mito 80 nM, Taxol 160 nM, Cisp 5 μM, Mel 1 μM, etc.), to the levels of expression of calumenin mRNA in non-resistant cell lines of the same tissue type.

FIG. 51 is a diagrammatic representation of a focused microarray chip showing the predetermined positions of the capture probes on the slide. The focused microarray of this figure is used for determinations of chemotherapeutic drug resistance in breast cancer cell samples.

FIG. 52 is a diagrammatic representation of a focused microarray chip showing the predetermined positions of the capture probes on the slide. The focused microarray of this figure is used for determinations of chemotherapeutic drug resistance in ovarian cancer cell samples.

### DETAILED DESCRIPTION OF THE INVENTION

The patent and scientific literature referred to herein establishes knowledge that is available to those of skill in the art. The issued US patents, allowed applications, published foreign applications, and references, including GenBank database sequences, that are cited herein are hereby incorporated by reference to the same extent as if each was specifically and individually indicated to be incorporated by reference.

1.1 General

An embodiment of the present invention in part provides methods and a device for diagnosing, detecting, or screening a cancer cell sample for chemotherapeutic drug resistance. The invention also allows for the improved clinical management of chemotherapeutic resistant tumors by providing a device that detects the expression level of genes identified as being markers for chemotherapeutic resistance. Furthermore, embodiments of the invention provide a focused microarray that allows for rapid identification of chemotherapeutic drug resistance in a cancer cell sample.

Accordingly, one aspect of the invention provides a focused microarray for diagnosis of chemotherapeutic drug resistance in a cancer cell. The microarray has a plurality of capture probes that bind marker genes isolated from the cancer cell. The nucleic acid capture probes are attached to a solid support at predetermined positions. For example, the focused microarray may include a solid support to which the nucleic acid capture probes are attached at predetermined positions. Useful solid supports include, but are not limited to, glass, metal alloy, silicon, and nylon. The support can be a slide derivatized with substances such as aldehydes, epoxies, poly-lysine, silanes, or amines, all of which are well known in the art and provide better deposition of capture probes to the slide.

As used herein, a “cancer cell” is a cell that shows aberrant cell growth, such as increased, uncontrolled cell growth. A cancer cell can be a hyperplastic cell, a cell from a cell line that shows a lack of contact inhibition when grown in vitro, a tumor cell when grown in vivo, or a cancer cell that is capable of metastasis in vivo. Non-limiting examples of cancer cells include melanoma, breast cancer, ovarian cancer, prostate cancer, sarcoma, leukemic retinoblastoma, hepatoma, myeloma, glioma, mesothelioma, carcinoma, leukemia, lymphoma, Hodgkin lymphoma, Non-Hodgkin lymphoma, promyelocytic leukemia, lymphoblastoma, and thymoma, and lymphoma cells, melanoma cells, sarcoma cells, leukemia cells, retinoblastoma cells, hepatoma cells, myeloma cells, glioma cells, mesothelioma cells, and carcinoma cells.

As used herein, the term “chemotherapeutic drug resistance” encompasses the development of resistance to a particular chemotherapeutic drug, class of chemotherapeutic drugs or multiple chemotherapeutic drugs by a cancer cell. Resistance can occur before or after treatment with a chemotherapy regime. The mechanism of development of chemotherapeutic drug resistance can occur by any means, such as by pathogenic means such as through infections, particularly viral infection. Alternatively, chemotherapeutic resistance can be conferred by a mutation or mutations in one or several genes located either chromosomally or extrachromosomally. In addition, chemotherapeutic drug resistance can be conferred by selection of a certain phenotype by exposure to the chemotherapeutic drug and then subsequent survival of the cell to the particular treatment. The above-mentioned mechanisms of chemotherapeutic drug resistance are known in the art.

As used herein, “chemotherapeutic drug” means a pharmaceutical compound that kills a damaged cell such as a cancer cell. Cell death can be induced by the chemotherapeutic drug through a variety of means including, but not limited to, apoptosis, osmolysis, electrolyte efflux, electrolyte influx, cell membrane permeablization, and DNA fragmentation. Exemplary non-limiting chemotherapeutic drugs are adriamycin, cisplatinum, taxol, melphalan, daunorubicin, dactinomycin, bleomycin, fluorouracil, teniposide, vinblastin, vincristine, methotrexate, mitomycin, docetaxel, chlorambucil, carmustine, mitoxantrone, and paclitaxel.

The term “focused microarray” as used herein refers to a device that includes a solid support with capture probe(s) affixed to the surface of the solid support. The capture probes are directed to the diagnosis of a specific condition, e.g., chemotherapeutic drug resistance. Typically, the support consists of silicon, glass, nylon or metal alloy. Solid supports used for microarray production can be obtained commercially from, for example, Genetix Inc. (Boston, Mass. Moreover, the support can be derivatized with a compound to improve nucleic acid association. Exemplary compounds that can be used to derivatize the support include aldehydes, poly-lysine, epoxy, silane containing compounds and amines. Derivatized slides can be obtained commercially from Telechem International (Sunnyvale, Calif.).

The term “marker genes” as used herein means any group of nucleic acid sequences, whether chromosomal or extrachromosomal, that is utilized by a cancer cell to produce a “gene product”, which can or cannot produce a phenotype in the cancer cell or the organism. As used herein, “gene product” means any biomolecule that is produced from a nucleotide sequence or could be produced from a nucleotide sequence. Gene products include, but are not limited to, pre-messenger RNA, messenger RNA, transfer RNA, heteronuclear RNA (“HnRNA”), ribosomal RNA, single-stranded DNA, double-stranded RNA, peptides and proteins. Extrachromosomal sources of nucleic acid sequences can include double-strand DNA viral genomes, single-stranded DNA viral genomes, double-stranded RNA viral genomes, single-stranded RNA viral genomes, bacterial DNA, mitochondrial genomic DNA, cDNA or any other foreign source of nucleic acid that is capable of generating a gene product.

For purposes of the invention, the term “capture probe” is intended to mean any agent capable of binding a gene product in a complex cell sample. Capture probes can be disposed on the derivatized solid support utilizing methods practiced by those of ordinary skill in the art through a process called “printing” (see, e.g., Schena et. al., (1995) Science, 270(5235): 467-470). The term “printing”, as used herein, refers to the placement of spots onto the solid support in such close proximity as to allow a maximum number of spots to be disposed onto a solid support. The printing process can be carried out by, e.g., a robotic printer. The VersArray CHIP Writer Prosystem (BioRad Laboratories) using Stealth Micro Spotting Pins (Telechem International, Inc, Sunnyvale, Calif.) is a non-limiting example of a chip-printing device that can be used to produce the focused microarray for this aspect. In certain embodiments, capture probes are nucleic acids (herein termed “nucleic acid capture probes”) that are attached to a solid support at predetermined positions.

In the case of nucleic acid capture probes, nucleic acid sequences that are selected for attachment to the focused microarray may correspond to regions of low homology between genes, thereby limiting cross-hybridization to other sequences. Typically, this means that the sequences show a base-to-base identity of less than or equal to 30% with other known sequences within the organism being studied. Sequence identity determinations can be performed using the BLAST research program located at the NIH website (www.ncbi.nlm.nih.gov/BLAST). Alternatively, the Needleman-Wunsch global alignment algorithm can be used to determine base homology between sequences (see Cheung et al., (2004) FEMS Immunol. Med. Micorbiol. 40(1): 1-9.). In addition, the Smith-Waterman local alignment can be used to determine a 30% or less homology between sequences (see Goddard et al., (2003) J. Vector Ecol. 28:184-9).

In another aspect, the invention provides methods for diagnosing chemotherapeutic drug resistance in a cancer cell. The methods can be practiced using a microarray composed of capture probes affixed to a derivatized solid support such as, but not limited to, glass, nylon, metal alloy, or silicon. Non-limiting examples of derivatizing substances include aldehydes, gelatin-based substrates, epoxies, poly-lysine, amines and silanes. Techniques for applying these substances to solid surfaces are well known in the art. In useful embodiments, the solid support can be comprised of nylon. Such slides are particularly useful when utilizing synthetic oligonucleotides. For example, nylon supports have been used to produce short oligonucleotides directly to the support (see, e.g., Liou et. al. (2004) BMC Urol. 4(1): 9).

In certain embodiments, the expression level of the marker genes in the cancer cell sample are compared to the expression level of the marker genes in a cancer cell of the same tissue type as the cancer cell sample that is sensitive to the chemotherapeutic drug or drugs. If the expression of at least one marker gene in the cancer cell is greater than the expression of the marker gene or genes in the sensitive cancer cell, then the cancer cell sample is drug-resistant. In some embodiments, the cancer cell sample is drug-resistant if the level of expression of in at least two or more of the plurality of marker genes in the cancer cell sample is greater than the level of expression of the same marker gene(s) in the non-drug-resistant cancer cell of the same tissue type.

The device can be incubated with labeled probes that correspond to any non-homologous sequences of the marker genes. Expression levels for the marker genes can be determined using techniques known in the art, such as, but not limited to, immunoblotting, quantitative RT-PCR, microarrays, RNA blotting, and two-dimensional gel-electrophoresis (see, e.g., Rehman et al. (2004) Hum. Pathol. 35(11):1385-91; Yang et al. (2004) Mol. Biol. Rep. 31(4):241-8). Such examples are not intended to limit the potential means for determining the expression of a gene marker in a breast cancer cell sample.

Non-homologous sequences pertaining to sequences identified in marker genes are used when using nucleic acid probes. Homology is determined by having a threshold homology of less than or equal to 30% for sequences utilized as probes. Homologies can be determined by the BLAST sequence alignment program located at the online site (www.ncbi.nlm.nih.gov/BLAST), the Needleman-Wunsch global alignment algorithm, or the Smith-Waterman local alignment. The device can be incubated with unlabeled probes and indirect methods of detection can be used to identify the expression level of marker genes in a cell sample. Protein expression levels are determined by methods that specifically recognize a particular sequence of amino acids in the protein.

Cell samples can be isolated from human tumor tissues using means that are known in the art (see, e.g., Vara et al. (2005) Biomaterials 26(18):3987-93; Iyer et al. (1998) J. Biol. Chem. 273(5):2692-7). For example, the cancer cell sample can be isolated from a human patient with breast cancer, or ovarian cancer, or lung cancer. Alternatively, cell samples can be obtained commercially from cell line sources as well (e.g., American Type Culture Collections, Mannassas, Va.).

As used herein, “breast cancer cell” is intended to mean a cell that originated from breast tissue that exhibits aberrant cell growth, such as increased cell growth. Likewise, the term “lung cancer cell” encompasses a cell that originated from lung tissue that exhibits aberrant cell growth, such as increased cell growth, and, “ovarian cancer cell” refers to a cell whose origins are from ovarian tissue and exhibits aberrant cell growth, such as increased cell growth.

Several non-limiting types of breast tissue from which cancer cells can be isolated including glandular, ductal, stromal, fibrous and lymphatic tissue. In addition, the cancer cell can be a metastatic cell isolated from bone, lymphatic tissue, blood, brain, lung, muscle, and skin. Breast, lung, or ovarian cancer cells can be isolated from a mammal such as a human, mouse, rat, horse, pig, guinea pig, or chinchilla. Exemplary non-limiting breast cancer cells include lobular neoplasia, ductal carcinoma in situ, infiltrating lobular carcinoma, infiltrating ductal carcinoma, tubular carcinoma, mucinous carcinoma, medullary carcinoma, phylloides tumor, inflammatory breast cancer, Paget's disease of the nipple, ductal carcinoma, and breast adenocarcinoma. Breast cancer cell lines are also available from common sources, such as the ATCC cell biology collections (American Type Culture Collections, Mannassas, Va.).

More specifically, the cancer cell can be isolated from several non-limiting types of lung tissue including glandular, bronchial, epithelial, diffuse lymphatic and bronchus-associated lymphatic. In addition, the cancer cell can be a metastatic cell isolated from bone, lymphatic tissue, blood, brain, breast, muscle, and skin. Lung cancer cells can be isolated from a mammal such as a human, mouse, rat, horse, pig, guinea pig, or chinchilla. Exemplary non-limiting lung cancer cells include non-small cell carcinoma, small cell carcinoma, large cell lung carcinoma, squamous cell lung cancer, and lung adenocarcinoma. Alternatively, lung cancer cell lines can be used and are available from common sources such as the ATCC cell biology collections.

In useful embodiments, housekeeping genes are used to normalize a signal on the focused microarray. As used herein, the term “housekeeping genes” refers to any gene that has relatively stable or steady expression during the life of a cell. Examples of housekeeping genes are well known in the art, such as, isocitrate lyase, acyltransferase, creatine kinase, TATA-binding protein, hypoxanthine phosphoribosyl transferase land guanine nucleotide binding protein, beta polypeptide 2-like 1 (see, e.g., Zhang et al. (2005) BMC Mol. Biol. 6:4). The housekeeping genes can be used to identify the proper signal level by which to compare the control signal and the drug-resistant signal.

In another aspect, the invention provides a method of diagnosing chemotherapeutic drug resistance in a cancer cell sample using an antibody microarray. To determine drug resistance, the level of protein expression of cell markers in the cancer cell sample is detected, and compared to the level of protein expression of the plurality of cell markers in a non-drug-resistant cancer cell of the same tissue type. An increased level of expression of cell markers in the cancer cell sample relative to the non-drug-resistant cancer cell is indicative of drug resistance.

As used herein, the term “antibody microarray” encompasses a solid surface to which antibodies are affixed to the surface by any means. The term “antibody microarray” is further meant to encompass devices that utilize immobilized antibodies as capture probes.

The term “cell marker” as used herein describes a protein found in or on the surface of cell that is produced from a sequence of nucleic acids located either chromosomally or extrachromosomally. Extrachromosomal nucleic acid sequences include double-strand DNA viral genomes, single-stranded DNA viral genomes, double-stranded RNA viral genomes, single-stranded RNA viral genomes, bacterial DNA, mitochondrial DNA, or any other non-nuclear or foreign source of nucleic acid that is capable of generating a gene product.

If the level of protein expression of at least two or three cell markers in the cancer cell is greater than the level of protein expression of the cell markers in the non-resistant cancer cell of the same tissue type, such increase in expression is indicative of drug resistance.

Proteins isolated from a cell can be labeled to allow detection of the level of expression of cell markers in a cancer cell sample. For example, the cell markers of the present aspect can be labeled for detection on the focused microarray using chemiluminescent tags affixed to amino acid side chains. Useful tags include, but are not limited to, biotin, fluorescent dyes such as Cy5 and Cy3, and radiolabels (see, e.g., Barry and Soloviev (2000) Proteomics. 4(12): 3717-3726). Tags can be affixed to the amino terminal portion of a protein or the carboxyl terminal portion of a protein (see, e.g., Mattison and Kenney, (2002) J. Biol. Chem., 277(13): 11143-11148; Berne et al., (1990) J. Biol. Chem. 265(32):19551-9). Indirect detection means can also be used to identify the cell markers. Exemplary but non-limiting means include detection of a primary antibody using a fluorescently labeled secondary antibody, or an antibody tagged with biotin such that it can be detected with fluorescently labeled streptavidin.

Antibodies for the production of capture probes can be generated by means well known in the art (see, e.g., Starling et al., (1982) Cancer Res. 42(8):3084-9; Ahn et al., (2004) J. Agric. Food Chem. 52(15):4583-94). Alternatively, polyclonal antibodies can be commercially obtained from non-limiting sources (such as Hy Laboratories Ltd. (Park Tamar Rehovot, Israel)). In addition, monoclonal antibodies can be commercially obtained from, but not limited to, sources such as A&G Pharmaceutical, Inc. (Columbia, Md.).

The antibodies can be attached to a solid support at predetermined positions to provide improved analysis of the levels of expression of a plurality of cell markers. In general, a protein microarray can be prepared by first modifying a solid support to allow for improved association of antibodies to the support. Depositing protein capture agents onto the modified substrate at pre-defined locations follows the modification of the support. Supports of choice for protein microarray applications can be organic, inorganic or biological. Some non-limiting, commonly used support materials include glass, plastics, and metals. Surfaces such as gold, PVDF, silica and polystyrene display high affinities for antibodies (see, e.g., Lal et. al., (2002) DDT (Suppl.) 7(18): S 143-S 149). The support can be transparent or opaque, flexible or rigid. In some cases, the support can be a porous membrane, e.g., nitrocellulose and polyvinylidene difluoride, and the protein capture agents are deposited onto the membrane by physical adsorption. To improve the robustness and reproducibility of the microarray signal, the protein capture agents can be immobilized onto a substrate through chemical covalent bonds.

It is important to note that the antibodies used in aspect of the present invention can be coupled to the surface of the microarray to improve the retention of the antibodies during processing. Coupling of the antibodies can thus improve the signal strength of the reaction and produce improved results. Common coupling agents include, but are not limited to, silanization using (3-mercaptopropyl)trimethoxysilane, agarose coating, and poly-L-lysine films. Additionally, recombinant antibodies can be engineered to include a tag facilitating coupling to the support. For example, a recombinant antibody having a histidine tag can be coupled to supports coated with nickel.

Another aspect of the invention provides a method of diagnosing chemotherapeutic drug resistance in a cancer cell sample. In this method, expression of a cell marker in the cancer cell is measured. This measurement can be measured by “slot blot” hybridization (see Ma et al., (2002) Methods Mol. Biol. 196:139-45) and quantitative RT-PCR can be used to determine the expression of marker genes in drug-resistant and drug-sensitive cells. Alternatively, RNA blotting can be used to screen drug-resistant and drug-sensitive cells for the expression of marker genes. RNA blot analysis is routine in the art (see, e.g., Ausubel, et al., Current Protocols in Molecular Biology, Vol. 1, pp. 4.2.1-4.2.9, John Wiley & Sons, Inc., 1996). Real-time quantitative PCR can be conveniently accomplished, e.g., using the commercially available ABI PRISMJ 7700 Sequence Detection System (available from PE-Applied Biosystems, Foster City, Calif.) according to manufacturer's instructions. Expression levels between drug-resistant and drug-sensitive cells can be compared using standard techniques known to those of skill in the art (see, e.g., Ma et al., (2002) Methods Mol. Biol. 196:139-45).

An antibody can be used to detect the level of expression of a cell marker. Antibody techniques such as immunoblotting and enzyme linked immunosorbent assay (ELISA) can be used, which are well-known in the art (see, e.g., Trampont et al., (2004) Hum. Pathol. 35(11):1353-9.). Additionally, antibodies can be conjugated to inert supports such as sepharose beads, cellulose beads or polystyrene beads. The bound cell markers are then eluted from the beads and analyzed by immunoblotting or ELISA. Alternatively, the antibody can be attached to a solid support composed of metal alloy, silica, PVDF membrane or nitrocellulose.

The cancer cell sample can be isolated from a human patient by a physician and tested for expression of marker genes using a focused microarray. Alternatively, the cancer cell sample can be isolated from an organism that develops a tumor or cancer cells including, but not limited to, mouse, rat, horse, pig, guinea pig, or chinchilla.

1.2. Cell Lines

A cancer cell can also be a cell line derived from a particular tissue. The term “cell line”, as used herein, refers to any cell that has been isolated from the tissue of a host organism and propagated by artificial means outside of the host organism. Such cell lines can be chemotherapeutic drug-resistant or chemotherapeutic drug-sensitive. A cell line can be isolated from tissues such as prostatic tissue, bone tissue, blood, brain tissue, lung tissue, ovarian tissue, epithelial tissue, breast tissue, and muscle tissue. A cell line can be derived, produced, or isolated from a cancer cell type, e.g., melanoma, breast cancer, ovarian cancer, prostate cancer, sarcoma, leukemic retinoblastoma, hepatoma, myeloma, glioma, mesothelioma, carcinoma, leukemia, lymphoma, Hodgkin lymphoma, Non-Hodgkin lymphoma, promyelocytic leukemia, lymphoblastoma, or thymoma. Exemplary, but non-limiting, cell lines are MCF-7, CEM, PC3, SKOV3, MDA, 2008, H460, T84, H69, HeLa, OVCAR3, and HCT-116. Cell lines can be commercially obtained, e.g., the ATCC cell biology collections (American Type Culture Collections, Mannassas, Va.). Alternatively, cell lines can be produced by methods known in the art (see, e.g., Griffin et. al., (1984) Nature 309(5963):78-82).

1.3. Capture Probes

A capture probe can be a nucleic acid sequence, which can be a full length sequence, fragments of full length sequences or synthesized oligonucleotides, that binds under physiological conditions to nucleic acids, e.g., by Watson-Crick base pairing (interaction between oligonucleotides and single-stranded nucleic acid) or by any other means including in the case of oligonucleotides binding to RNA, pseudoknot formation. Capture probes can be composed of DNA, RNA, or both. Nucleic acid capture probes are complementary to cDNA or cRNA sequences obtained from pre-messenger RNA, messenger RNA, transfer RNA, heteronuclear RNA (“HnRNA”), ribosomal RNA, bacterial RNA, mitochrondrial RNA or viral RNA.

“Nucleic acid” refers to a polymer comprising 2 or more nucleotides and includes single-, double-, and triple-stranded polymers. “Nucleotide” refers to both naturally occurring and non-naturally occurring compounds and comprises a heterocyclic base, a sugar, and a linking group, such as a phosphate ester. For example, structural groups may be added to the ribosyl or deoxyribosyl unit of the nucleotide, such as a methyl or allyl group at the 2′-O position or a fluoro group that substitutes for the 2′-O group. The linking group, such as a phosphodiester, of the nucleic acid may be substituted or modified, for example with methyl phosphonates or O-methyl phosphates. Bases and sugars can also be modified, as is known in the art. “Nucleic acid,” for the purposes of this disclosure, also includes “peptide nucleic acids” in which native or modified nucleic acid bases are attached to a polyamide backbone.

The length of a nucleic acid capture probe is less than or equal to the full length of an RNA product generated by a gene sequence so long as the capture probe sequence is complementary to the marker gene sequences and shows less than or equal to 30% homology to other known sequences within the organism being studied. Importantly, nucleotide sequences of between about 50 and about 150 bases in length provide optimal gene expression resolution, while reducing background, non-specific hybridization that occurs with nucleic acid sequences of full length genes (Cheng-Chung Chou et. al., Nucleic Acids Res. Jul. 08, 2004;32(12):e99). The length of the oligonucleotide can be between about 55 and about 145 bases, between about 60 and about 140 bases, between about 65 and about 135 bases, between about 70 and about 130 bases, and/or between about 75 and about 125 bases. However, sequences greater than about 150 base pairs and less than about 50 base pairs are still effective capture probes and can be used to identify marker genes.

Nucleic acid capture probes can be obtained by any means known in the art. For example, they can be synthetically produced using the Expedite™ Nucleic Acid Synthesizer (Applied Biosystems, Foster City, Calif.) or other similar devices (see, e.g., Applied Biosystems, Foster City, Calif.). Synthetic oligonucleotides also can be produced using methods well known in the art such as maskless photolithography (see, e.g., Nuwaysir et. al., (2002) Gen. Res. 12:1749-1755), phosphoramidite methods (see, e.g., Pan et. al., (2004) Biol. Proc. Online. 6:257-262), H-phosphonate methodology (see, e.g., Agrawal et. al., (1987) Tetrahedron Lett. 28(31): 3539-3542) and phosphite trimester methods (Nucleic Acids Res. (1984), 12: 4539; (1983) Tetrahedron Lett. 24: 5843).

It should be recognized that the capture probes can be attached to linkers such as 3′ amino linkers or 5′ amino linkers without changing the functionality of the capture probes. Also, additional nucleotides can be attached to the 3′ end of a capture probe during nucleic acid synthesis for the purpose of acting as a linker. Generally, linkers can be attached to capture probes to improve the binding efficiency of the capture probe to the target nucleic acid. The procedures used to attach various linker moieties to capture probes are recognized in the art (see, e.g., Steinberg et al., (2004) Biopolymers 73(5):597-605).

Additionally, the capture probes can be modified in a number of ways that would not compromise their ability to hybridize to a particular nucleic acid sequence. Modifications to the nucleic acid structure can include synthetic linkages such as alkylphophonates, phosphoramidites, carbamates, carbonates, phosphate esters, acetamide, and carboxymethyl esters (see, e.g., Agrawal et. al., (1987) Tetrahedron Lett. 28:3539-3542; Agrawal et. al., (1988) PNAS (USA) 85:7079-7083; Uhlmann et. al., (1990) Chem. Rev. 90:534-583; Agrawal et. al., (1992) Trends Biotechnol. 10: 152-158). Additionally, nucleic acid modifications include internucleoside phosphate linkages such as chlesteryl linkages or diamine compounds of varying numbers of carbon residues between the amino groups and terminal ribose. Other modifications of capture probes include changes to the sugar moiety such as arabinose or 3′, 5′ substituted nucleic acids having a sugar attached at its 3′ and 5′ ends through a chemical group other than a hydroxyl group. These modifications can be added to a capture probe sequence without compromising hybridization efficiency (see, e.g., Valoczi et. al., (2004) Nucleic Acids Res. 32(22):e175; Zatsepin et. al., (2004) IUBMB Life. 56(4): 209-214). Therefore, modifications that do not compromise the hybridization efficiency of the capture probe are within the scope of the invention.

Alternatively, a capture probe can be a protein capable of binding a biological macromolecule such as a protein, nucleic acid, simple carbohydrate, complex carbohydrate, fatty acid, lipoprotein, and/or triacylglyceride. The mechanisms of binding to a target molecule include, e.g., hydrogen bonding, Van der Waals attractions, covalent bonding, ionic bonding, or hydrophobic interactions. Exemplary protein capture probes include natural ligands of a receptor, hormones, antibodies, and portions thereof.

For example, when the capture probe is an antibody, the methods of the invention allow for the detection of protein expression using expression detection systems, such as immunoblotting. The use of antibodies to detect changes in protein expression is well recognized in the art, and represents a tool for determining increases in the levels of expression of the cell markers in chemotherapeutic resistant cells. In these cases, the antibody can be, without limitation, a polyclonal antibody, a monoclonal antibody, a chimeric antibody, a humanized antibody, a genetically engineered antibody, a bispecific antibody (where one of the specificities of the bispecific antibody binds to a cell marker), antibody fragments (including, but not limited to, “Fv,” “F(ab′)₂,” “F(ab),” “Dab”); and single chains representing the reactive portion of an antibody (“SC-Mab”). Proteins and antibodies can be obtained commercially or made by any known means (see, e.g., Coligan et al., Current Protocols in Immunology, John Wiley and Sons, New York City, N.Y., (1991); Jones et al., (1986) Nature 321: 522-525; Marx, (1985) Science 229: 455-456; Rodwell, (1989) Nature 342: 99-100).

In certain methods of practicing the invention, antibodies can be part of an antibody array where they are immobilized on a solid support such as a bead or flat surface similar to a slide. An antibody microarray can determine the cell marker expression of a chemotherapeutic drug-resistant cancer cell sample and the cell marker expression of a chemotherapeutic drug-sensitive control cell of the same tissue type. Each capture probe binds a target that has been labeled. The slide has one or more spots, each of which contains antibodies specific for a particular cell marker. The focused microarray can identify cell markers with increased expression in chemotherapeutic drug-resistant cancer cells.

1.4. Marker Genes

Marker gene expression is used to identify the indicia of chemotherapeutic drug resistance. Marker genes can be obtained by isolation from a cancer cell sample by mechanisms available to one of ordinary skill in the art (see, e.g., Ausubel et. al., Current Protocols in Molecular Biology, Wiley and Sons, New York, N.Y., 1999). Isolation of nucleic acids from the cancer cell sample allows for the generation of target molecules that can be captured by the capture probes on the surface of the microarray, providing a means for determining the expression level of the marker genes in the cancer cell sample as described below. Isolation of proteins from the cancer cell sample allows for the generation of target molecules for the capture probes, as well. The marker genes can be isolated from a tissue sample isolated from a human patient. Alternatively, marker genes are isolated in the form of RNA transcripts. Methods of RNA isolation are taught in, for example, Ausubel et al., Current Protocols in Molecular Biology, Vol. 1, pp. 4.1.1-4.2.9 and 4.5.1-4.5.3, John Wiley & Sons, Inc., (1993).

Useful marker genes detected to determine the existence of chemotherapeutic drug resistance include breast cancer resistance protein (BCRP) (gi # 12414050) 1-68 bp of cds; multidrug resistance-associated protein 1 (MRP-1) (gi# 9955961) 303-370 bp of cds; multidrug resistance-associated protein 1 (MRP-1)(gi# 9955961) 4501-4568 bp of cds; P-glycoprotein 1 (Pgp 1) (gi# 7669470) 201-268 bp of cds; Pgp 11 (gi# 7669470) 3061-3128 bp of cds; Fatty acid binding protein 7 (FABP7) (gi# 16950660) 330-398 bp of cds; Lung resistant protein (gi#19577289) 2400-2468 bp of cds; topoisomerase IIα (gi# 19913405) 4500-4568 bp of cds; Fatty acid binding protein 3 (FABP3) (gi# 10938020) 334-402 bp of cds; cathepsin β (gi# 22538429) 942-1010 bp of cds; p53 (gi# 35213) 1073-1141 bp of cds; Heat shock protein 90 (HSP90) (gi# 184422) 2100-2168 bp of cds; Heat shock protein 60 (HSP60) (gi# 14730099) 1801-1868 bp of cds; γ-actin (gi# 11038618) 1000-1068 bp of cds; Vimentin (gi# 4507894) 1-68 bp of cds; vimentin (gi# 4507894) 1261-1328 bp of cds; BIP (gi# 6470149) 1631-1698 bp of cds; p-40 (gi# 4502100) 1-68 bp of cds; annexin-1/p-40 (gi# 4502100) 823-890 bp of cds; nucleophosmin (gi# 10835062) 543-610 bp of cds; nucleophosmin (gi# 10835062) 813-880 bp of cds; Heat shock 70 kDa protein 8 (HSC70)(gi# 5729876) 1451-1518 bp of cds; Heat shock 70 kDa protein 8 (HSC70) (gi# 5729876) 1645-1712 bp of cds; galectin-1 (gi# 6006015) 341-408 bp of cds; Heat shock protein 27 (HSP27) (gi# 4996892) 61-128 bp of cds; ubiquitin C-term hydrolase isozyme L1 (UCHL-1) (gi# 18558293) 213-280 bp of cds; ubiquitin C-term hydrolase isozyme L1 (UCHL-1) (gi# 18558293) 471-538 bp of cds; ATP synthase β (gi# 179280) 1033-1200 bp of cds; prosolin (gi# 13518023) 351-418 bp of cds; thioredoxine peroxidase 1 (gi# 440307) 529-597 bp of cds; β-tubulin (gi# 3387928) 400-468 bp of cds; guanine nucleotide binding protein, β polypeptide 3 (GNBP β3) (gi# 183412) 350-398 bp of cds; MB-COMT (gi# 6466451) 101-168 bp of cds; EZRIN (gi# 21614498) 1011-1078 bp of cds; KAP-1 (gi# 1699026) 1-68 bp of cds; UMP-CMP kinase (gi# 5730475) 391-458 bp of cds; alternative splicing factor (ASF-2) (gi# 179073) 811-878 bp of cds; pyrophosphatase inorganic (gi# 12735403) 533-600 bp of cds; GST-π α chain (gi# 31947) 565-633 bp of cds; ATP synthase D (gi# 5453558) 213-280 bp of cds; chromobox homolog 3 (CBX3) (gi# 15082257) 31-98 bp of cds; protein disulfide isomerase precursor (PDI) (gi# 20070124) 543-610 bp of cds; dimethylarginine dimethylaminohydrolase 1 (DADEH1) (gi# 6912327) 399-456 bp of cds; dimethylarginine dimethylaminohydrolase 1 (DADEH1) (gi# 6912327) 651-718 bp of cds; Elongation factor 2 (EF2) (gi# 181968) 833-900 bp of cds; α-enolase (gi# 2661038) 943-1010 bp of cds; eukaryotic translation factor 3 subunit 2 (ETF3-subunit 2) (gi# 4503512) 833-900 bp of cds; heterogenous nuclear ribonucleoprotein F (HnRNP) (gi# 14141150) 771-838 bp of cds; tropomyosin 2 β (gi# 20070122) 550-617 bp of cds; eukaryotic translation initiator factor 4B (EIF 4B) (gi# 4503532) 901-968 bp of cds; hepatoma derived growth factor (gi# 4758515) 393-460 bp of cds; keratin type II cytoskeletal (gi# 12737278) 1171-1238 bp of cds; prohibitin (gi# 6031190) 713-780 bp of cds; solute carrier family 9 isoform 3 regulatory factor 1 (slc9A3R1) (gi# 4759139) 631-738 bp of cds; 5C5-2 (gi# 4324471) 141-208 bp of cds; protein disulfide isomerase ER-60 precursor (PDI-ER60) (gi# 1208427) 833-900 bp of cds; β-spectrin (gi# 338439) 3100-3168 bp of cds; β-spectrin (gi# 338439) 4000-4068 bp of cds; Superoxide dismutase (SOD) (gi# 4507148) 391-458 bp of cds; caspase recruitment domain protein 14 (gi# 13653996) 895-968 bp of cds; N-ethylmaleimide-sensitive factor attachment protein γ (NEM-sensitive factor attachment protein γ) (gi# 4505330) 732-800 bp of cds; fatty acid synthase (FAS) (gi# 4758341) 1-68 bp of cds; fatty acid synthase (FAS) (gi# 4758341) 7233-7300 bp of cds; triosephosphate isomerase (TPI) (gi# 339840) 400-467 bp of cds; Rad23 homolog β (gi# 19924138) 900-968 bp of cds; L-Plastin (gi# 16307447) 1600-1668 bp of cds; α-tubulin (gi# 3420928) 1288-1356 bp of cds; fatty acid binding protein, epidermal (E-FABP) (gi# 4557580) 1-68 bp of cds; fatty acid binding protein, epidermal (E-FABP) (gi# 4557580) 341-408 bp of cds; “similar to stratifin” (gi# 16306736) 314-382 bp of cds; cathepsin δ (gi# 18577791) 411-478 bp of cds; p16INK4a (gi# 16753086) 1-68 bp of cds; p6INK4a (gi# 16753086) 50-118 bp of cds; adenine phosphoribosyltransferase (APRT) (gi# 4502170) 100-168 bp of cds; calumenin (gi# 14718452) 880-943 bp of cds; ACRABP-II (gi# 6382069) 481-548 bp of cds; keratin 19 (gi# 40217850) 141-208 bp of cds; c-erb/HER-2/neu (gi# 4758297) 1981-2048 bp of cds; MYL16 (gi# 17986259) 252-319 bp of cds; interleukine 18 precursor (gi# 14210476) 431-498 bp of cds; cytokeratin 7 (gi# 3008955) 1461-1528 bp of cds.

The marker genes derived from the cancer cell sample can be further utilized to produce the targets-of-interest (herein termed “nucleic acid probes”) for the capture probes. As used herein a “nucleic acid probes” is defined as a nucleic acid capable of binding to a target nucleic acid of complementary sequence through one or more types of chemical bonds, usually through complementary base pairing, usually through hydrogen bond formation. As used herein, a nucleic acid probe may include natural (i.e. A, G, U, C, or T) or modified (7-deazaguanosine, inosine, etc.) bases. In addition, a linkage other than a phosphodiester bond may join the bases in probes, so long as it does not interfere with hybridization. Thus, nucleic acid probes may be peptide nucleic acids in which the constituent bases are joined by peptide bonds rather than phosphodiester linkages. The nucleic acid probes may be prepared by converting the RNA to cDNA using known methods (see, e.g., Ausubel et. al., Current Protocols in Molecular Biology Wiley 1999, pp.). The probes can also be cRNA (see, e.g., Park et. al., (2004) Biochem Biophys Res Commun. 325(4):1346-52).

Nucleic acid probes can be produced from synthetic methods such as phosphoramidite methods, H-phosphonate methodology, and phosphite trimester methods. Nucleic acid probes can also be produced by PCR methods. Such methods produce cDNA and cRNA sequences complementary to the mRNA.

The nucleic acid probes can be detectably labeled. As used herein, “detectably labeled” means that a probe is operably linked to a moiety that is detectable. By “operably linked” is meant that the moiety is attached to the probe by either a covalent or non-covalent (e.g., ionic) bond. Methods for creating covalent bonds are known (see general protocols in, e.g., Wong, S. S., Chemistry of Protein Conjugation and Cross-Linking, CRC Press 1991; Burkhart et al., The Chemistry and Application of Amino Crosslinking Agents or Aminoplasts, John Wiley & Sons Inc., New York City, N.Y., 1999).

According to the invention, a “detectable label” is a moiety that can be sensed. Such labels can be, without limitation, fluorophores (e.g., fluorescein (FITC), phycoerythrin, rhodamine), chemical dyes, or compounds that are radioactive, chemoluminescent, magnetic, paramagnetic, promagnetic, or enzymes that yield a product that may be colored, chemoluminescent, or magnetic. The signal is detectable by any suitable means, including spectroscopic, photochemical, biochemical, immunochemical, electrical, optical or chemical means. In certain cases, the signal is detectable by two or more means. In certain embodiments, nucleic acid labels include fluorescent dyes, radiolabels, and chemiluminescent labels, which are examples that are not intended to limit the scope of the invention (see, e.g., Yu, et al., (1994) Nucleic Acids Res. 22(16): 3226-3232; Zhu, et al., (1994) Nucleic Acids Res. 22(16): 3418-3422).

For example, nucleotides of nucleic acid probes may be conjugated to Cy5/Cy3 fluorescent dyes. These dyes are frequently used in the art (see, e.g., Yang et al., (2005) Clin Cancer Res. 11(2 Pt 1):612-20). The fluorescent labels can be selected from a variety of structural classes, including the non-limiting examples such as 1- and 2-aminonaphthalene, p,p′diaminostilbenes, pyrenes, quaternary phenanthridine salts, 9-aminoacridines, p,p′-diaminobenzophenone imines, anthracenes, oxacarbocyanine, marocyanine, 3-aminoequilenin, perylene, bisbenzoxazole, bis-p-oxazolyl benzene, 1,2-benzophenazin, retinol, bis-3-aminopridinium salts, hellebrigenin, tetracycline, sterophenol, benzimidazolyl phenylamine, 2-oxo-3-chromen, indole, xanthen, 7-hydroxycoumarin, phenoxazine, salicylate, strophanthidin, porphyrins, triarylmethanes, flavin, xanthene dyes (e.g., fluorescein and rhodamine dyes); cyanine dyes; 4,4-difluoro-4-bora-3a,4a-diaza-s-indacene dyes and fluorescent proteins (e.g., green fluorescent protein, phycobiliprotein).

Other useful dyes are chemiluminescent dyes and can include, without limitation, biotin conjugated DNA nucleotides and biotin conjugated RNA nucleotides. Labeling of nucleic acid probes can be accomplished by any means known in the art, e.g., CyScribe™ First Strand cDNA Labeling Kit (#RPN6200, Amersham Biosciences, Piscataway, N.J.).

The label can be added to the target nucleic acid(s) prior to, or after the hybridization. So called “direct labels” are detectable labels that are directly attached to, or incorporated into, the target nucleic acid prior to hybridization. In contrast, so called “indirect labels” are joined to the hybrid duplex after hybridization. Often, the indirect label is attached to a binding moiety that has been attached to the target nucleic acid prior to the hybridization. Thus, for example, the target nucleic acid may be biotinylated before the hybridization. After hybridization, an avidin-conjugated fluorophore binds the biotin bearing hybrid duplexes providing a label that is easily detected. (See, e.g., Laboratory Techniques in Biochemistry and Molecular Biology, Vol. 24: Hybridization With Nucleic Acid Probes, P. Tijssen, ed. Elsevier, N.Y., (1993)).

The target molecules of the present invention can also be proteins isolated or derived from the cancer cell sample. The proteins may be subsequently detectably labeled by being operably linked to a moiety that is detectable. Proteins have been detectably labeled by methods that have been discussed previously (see, e.g., Macbeth, (2002) Nature Genet. 32 (Suppl.): 526-532). Exemplary detectable labels of proteins include, but are not limited to, fluorescent dyes, radiolabels (see, e.g., Jona et. al., (2003) Curr. Opin. Mol. Therap. 5(3): 271-277) and chemiluminescent labels (see, e.g., Bacarese-Hamilton et. al., (2003) Curr. Opin. Mol. Therap. 5(3): 278-284). Commonly, fluorescent dyes include the Cy3/Cy5 protein dyes. Typical chemiluminescent labels include biotin hydrazides and biotin hydroxylamine.

Alternatively, the protein probe can be unlabeled. The labeled detection molecule can be an antibody unattached to the solid support, but capable of recognizing the probe. The unattached antibody can be conjugated to a label such as a radiolabel, chemilurninescent label or fluorescent dyes. Commonly, fluorescent dyes include the Cy3/Cy5 protein dyes. Typical chemiluminescent labels include, but are not limited to, biotin hydrazides and biotin hydroxylamine.

To demonstrate the methods according to the invention and the focused microarray, focused microarrays were prepared as described above and tested using the methods described above for their ability to diagnose chemotherapeutic drug resistance in various cancer cell samples.

The oligonucleotides tested on the nucleic acid focused microarray have been described above. Oligonucleotides attached to the focused microarray were designed so as to an overall thermal melting point of 76.97±3.72° C. at a sodium concentration of 50 mM. Normalization of signal was performed using Arabidopsis thaliana chlorophyll synthetase G4 positive control DNA. Statistical analysis was performed using a log transformation of the ratio data on all experiments, and a Student T test was used to determine statistically significant results. A difference in expression level is found when the ratio of Cy5 to Cy3 is greater than 1.5. Statistically significant differences in expression between samples were found if the p value was lower than 0.05.

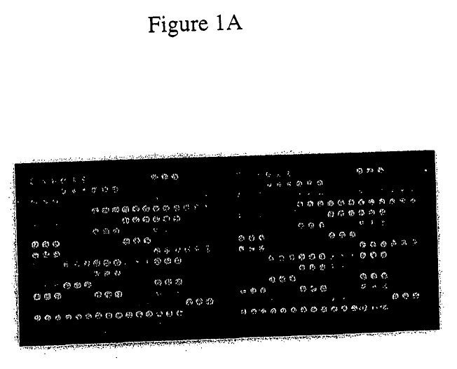

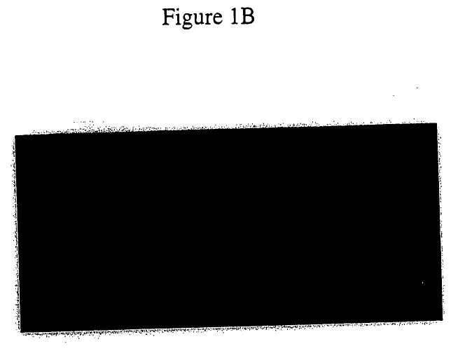

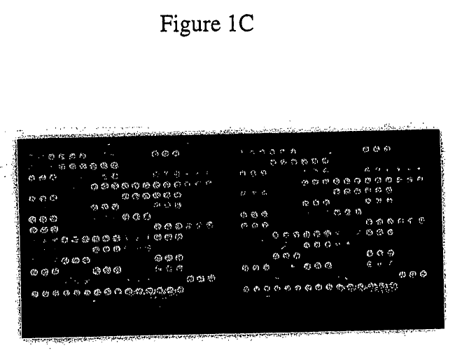

To test the focused microarray's capacity to determine increased expression of a nucleic acid marker gene in a chemotherapeutic drug-resistant cancer cell, capture probes were disposed on a microarray. The sequences represented regions of within each marker gene that had homologies to other genes of less than 30%. The capture probes consisted of sequence lengths of 68 bases and melting temperatures averaging 76.97° C.±3.72° C. Thus, hybridizations between capture probes and marker gene targets would be specific, and uniform hybridization was expected between capture probes and specific targets. By maintaining the average hybridization temperature within a limited range amongst capture probes, the clinician is able to obtain similar intensity results between spots on the chip. In particular, this was found in control experiments utilizing cell samples obtained from MDA cell lines sensitive to mitoxantrone and MDA cell lines resistant to mitoxantrone (FIGS. 1A and 1C).

During the testing of the focused microarray, cell lines were chosen that represented several tissues (see Table 1).

TABLE 1
Cell Lines Used for Focused Microarray Tests

| Drug-Sensitive | Drug-Resistant (Drug Concentration) |
|---|---|
| MCF7 | MCF7/adriamycin-resistant (4.8 μM) |
| | MCF7/vinblastin-resistant (10 nM) |
| | MCF7/mitoxantrone-resistant (10 nM) |
| | MCF7/vincristine-resistant (78 nM) |
| MDA | MDA/adriamycin-resistant (80 nM) |
| | MDA/adriamycin-resistant (400 nM) |
| | MDA/mitoxantrone-resistant (80 nM) |
| | MDA/taxol-resistant (2.5 nM) |
| SKOV3 | SKOV3/adriamycin-resistant (31.25 nM) |
| | SKOV3/adriamycin-resistant (62.5 nM) |
| | SKOV3/vinblastin-resistant (1.0 μM) |
| | SKOV3/taxol-resistant (160 nM) |
| | SKOV3/taxol-resistant (320 nM) |
| | SKOV3/cisplatinum-resistant (80 μM) |
| 2008 | 2008/adriamycin-resistant (125 nM) |
| | 2008/adriamycin-resistant (250 nM) |
| | 2008/taxol-resistant (160 nM) |
| | 2008/taxol-resistant (320 nM) |
| | 2008/cisplatinum-resistant (5 μM) |
| | 2008/cisplatinum-resistant (10 μM) |
| OVCAR3 | OVCAR3/taxol-resistant (2 nM) |
| PC3 | PC3/melphalan-resistant (1 μM) |
| T84 | T84/vincristine-resistant (62.5 nM) |
| | T84/vincristine-resistant (125 nM) |
| | T84/vincristine-resistant (250 nM) |
| | T84/cisplatinum-resistant (5 μM) |
| HCT-116 | HCT-116/vincristine-resistant (16 nM) |
| | HCT-116/vincristine-resistant (32 nM) |
| H69 | H69/adriamycin-resistant (800 nM) |
| H460 | H460/adriamycin-resistant (120 nM) |
| | H460/taxol-resistant (40 nM) |
| | H460/taxol-resistant (80 nM) |

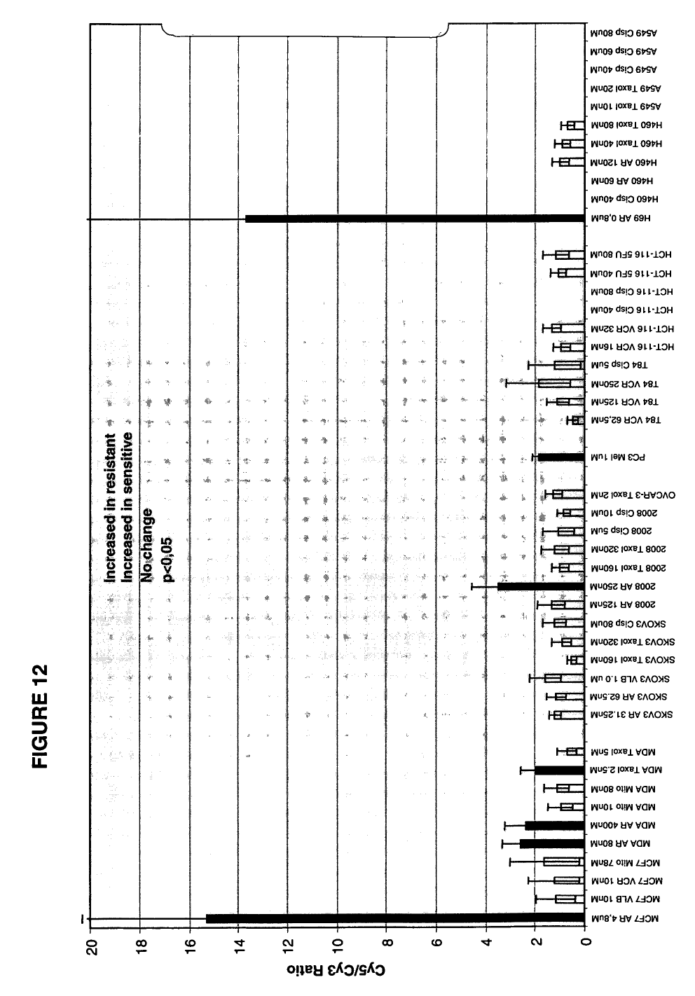

In particular, the MDA and MCF7 cell lines are epithelial adenocarcinomas isolated from breast tissue. The focused microarray was hybridized with a mixture of labeled-cDNA produced from cell sample RNA obtained from the drug-resistant breast cancer cell lines and the drug-sensitive breast cancer cell lines. The hybridization of a mixture of cDNA represented the comparison of expression between the drug-sensitive cell sample and the drug-resistant cell sample. Hybridization of the cDNA sample was followed by scanning of the microarray to determine the differences in expression between the drug-resistant cell line and the drug-sensitive cell line. The hybridization of the marker gene targets to the capture probes on the microarray established that the adriamycin-resistant breast cancer cell lines had increased expression of certain marker genes. For example, the microarray data clearly indicates that the spot on the microarray corresponding to the marker gene annexin-1 is increased in expression in adriamycin-resistant breast cancer cell lines (FIG. 12). A Student t-test showed that the MCF7 and the MDA adriamycin-resistant cell lines had statistically significant increased expression of annexin-1 (FIG. 12). The bar graph of FIG. 12 further shows that other cell lines that had increased expression of annexin-1 mRNA in cells resistant to other chemotherapeutic drugs as well (FIG. 12). These results indicate that increased expression of annexin-1 is identified in certain adriamycin-resistant cells (FIG. 12). Thus, a cancer cell sample that has increased expression of annexin-1 over a adriamycin-sensitive control sample is, more likely than not, adriamycin-resistant.

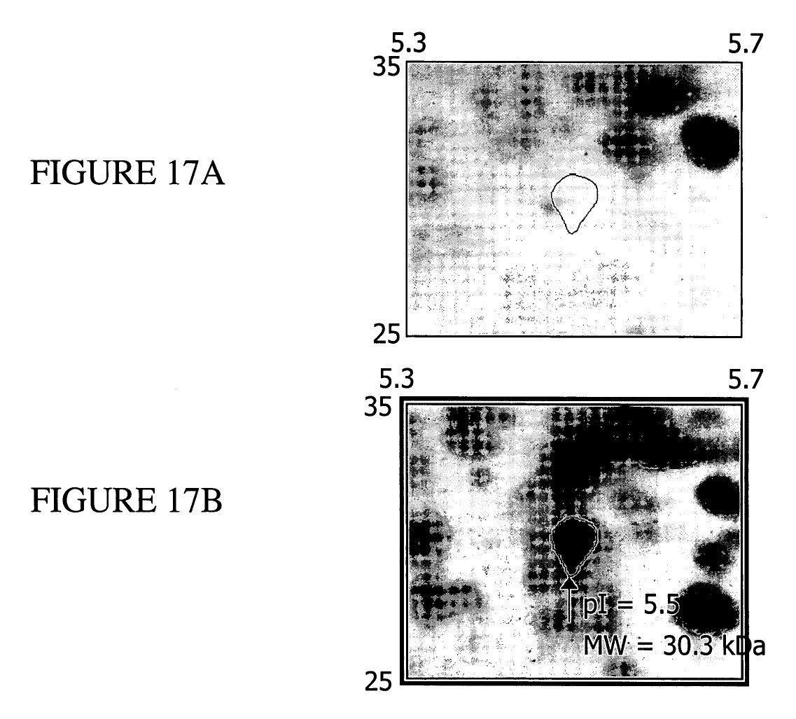

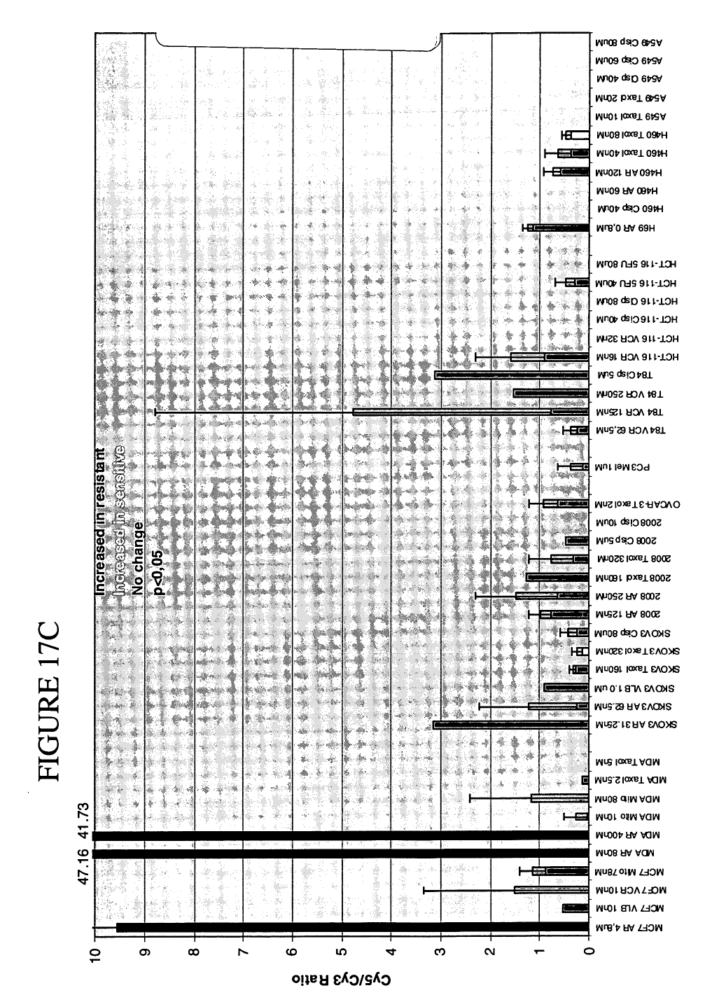

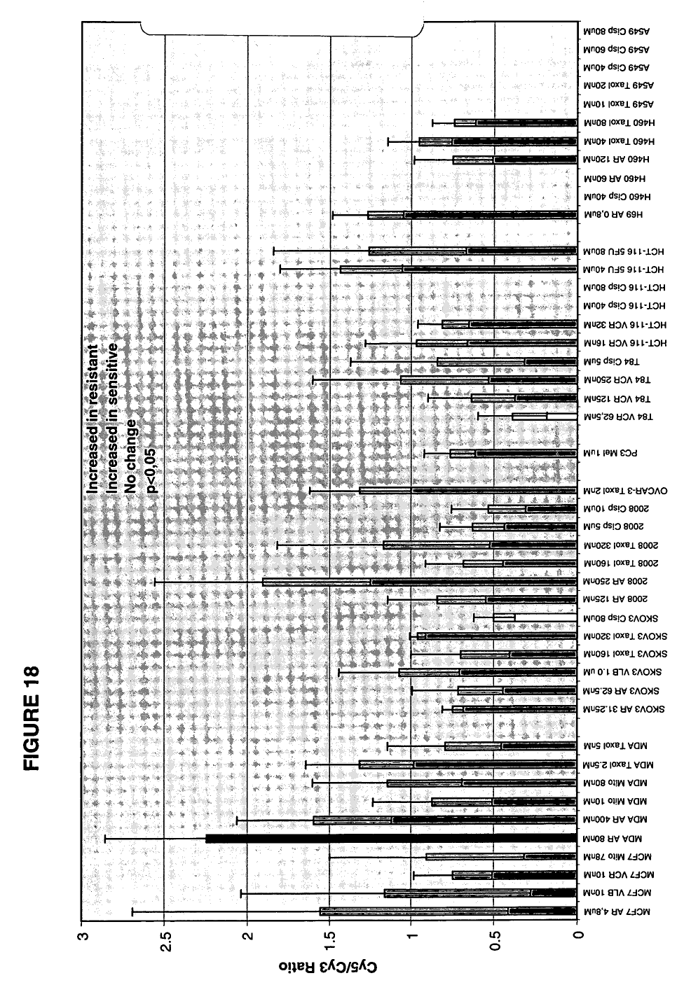

In addition to annexin-1, increased mRNA expression in adriamycin-resistant MCF7 and adriamycin-resistant MDA cell lines was found for UCHL-1, ezrin and “similar to stratifin” (FIGS. 17C, 22C, and 47C). These results demonstrate that increased expression findings are not limited to a particular marker gene, but rather are found in a plurality of marker genes that can be used for diagnostic screening of adriamycin resistance in breast cancer cell samples. Furthermore, the mRNA expression levels found were significantly increased over resistant cells, particularly for UCHL-1 (FIG. 17C).

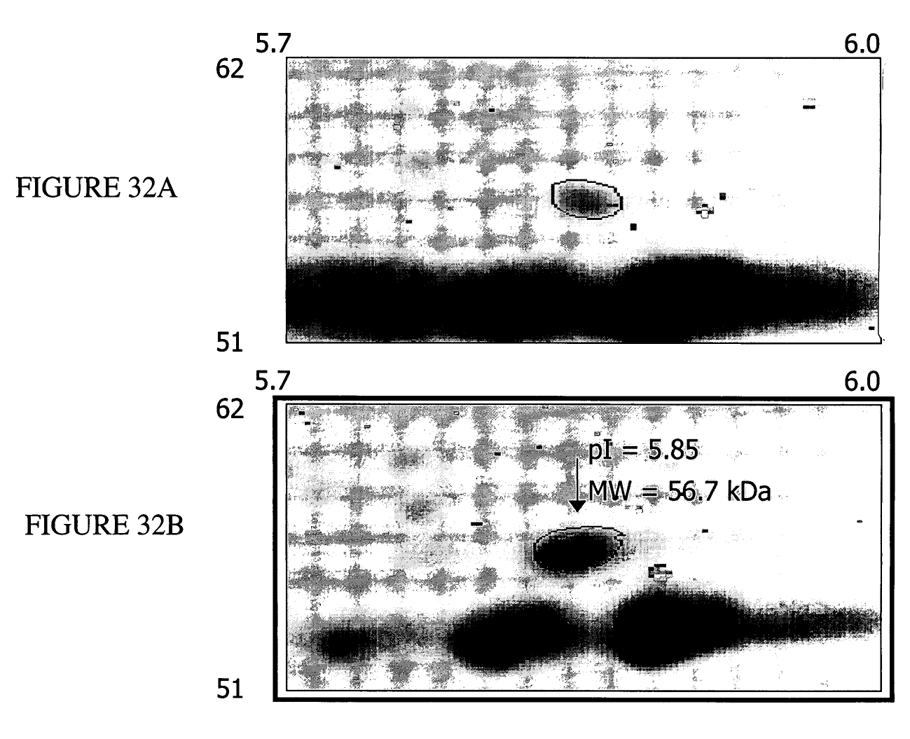

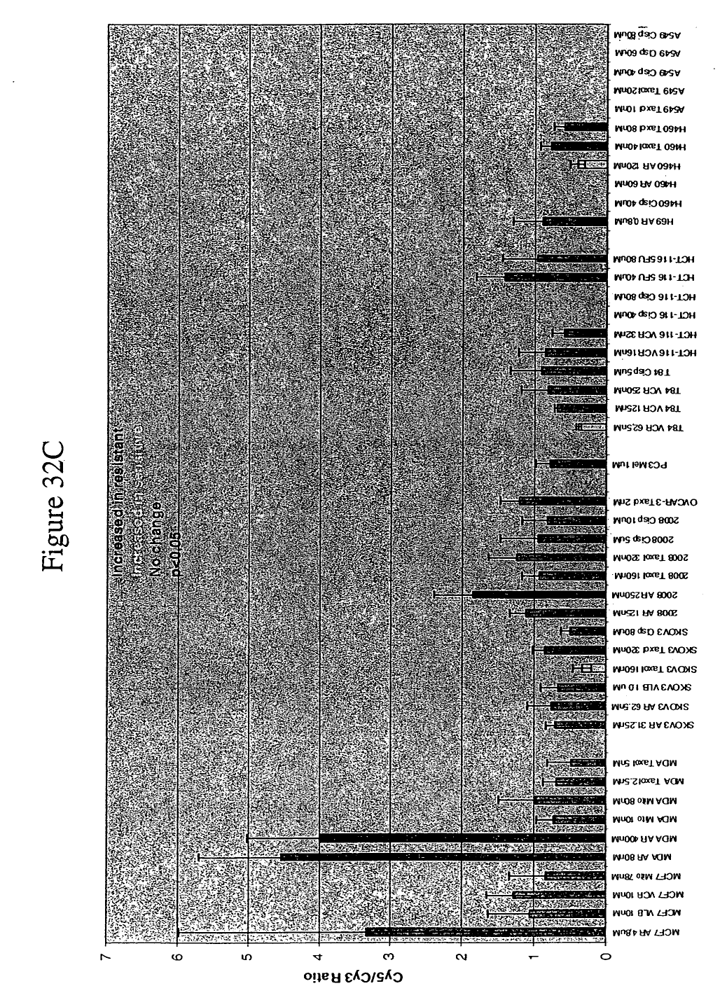

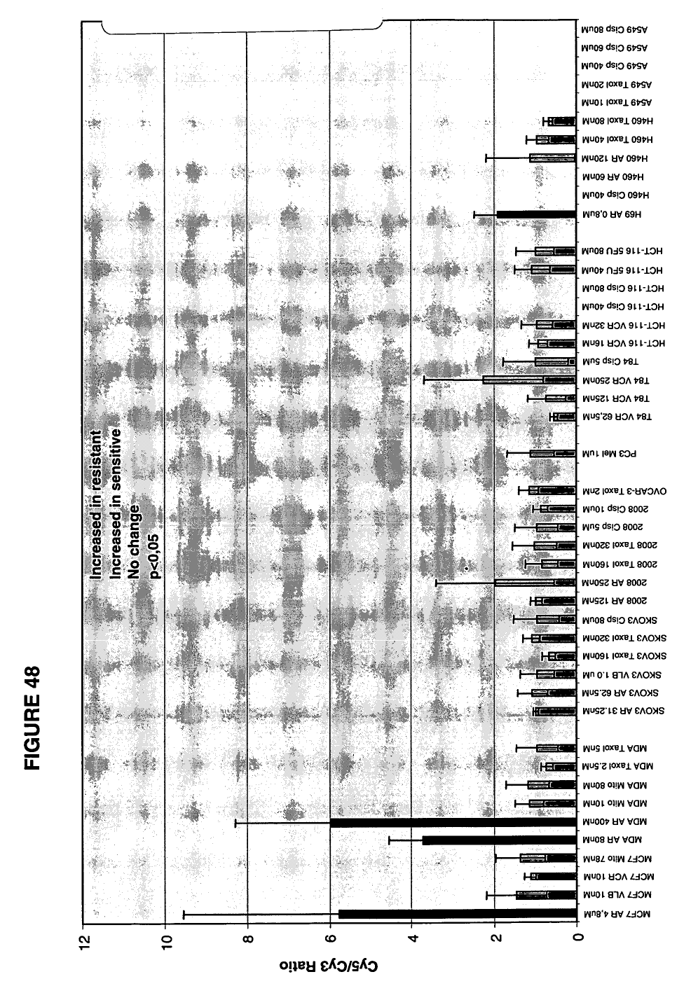

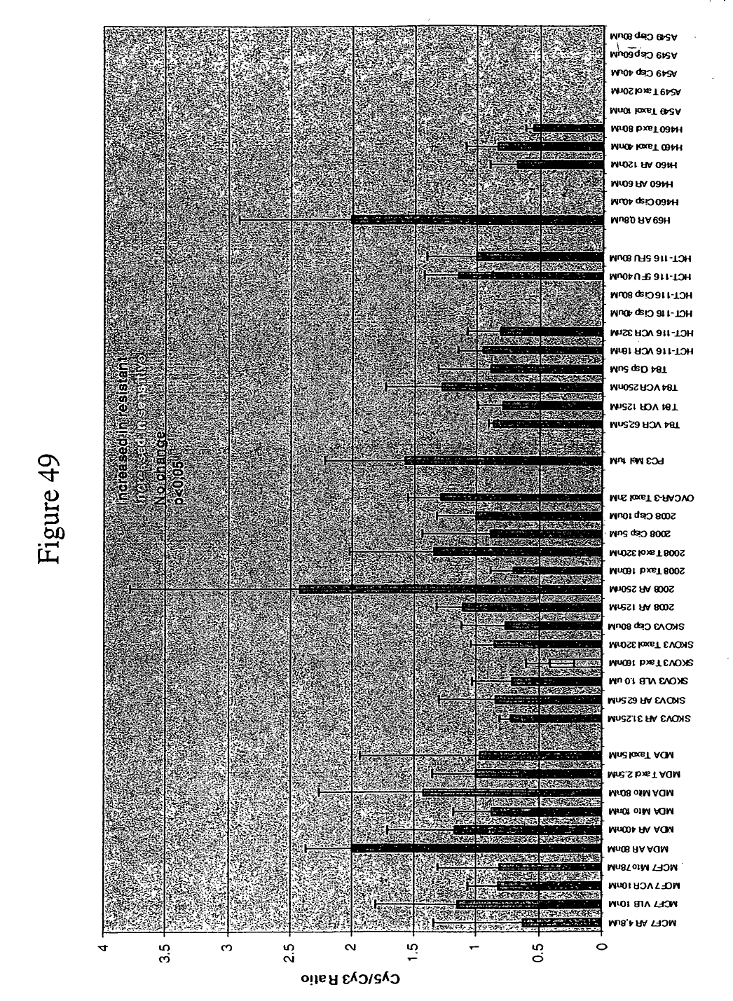

The marker genes E-FABP, HnRNP and p16INK4a are also increased in expression in resistant breast cancer cell lines compared to adriamycin-sensitive cell lines (FIG. 32C, 46C, and 48). As indicated above, increased expression of these marker genes in adriamycin-resistant cell lines established that several marker genes could be identified reliably by the microarray in a breast cancer cell sample. For example, adriamycin-resistant breast cancer cell samples showed increased expression of p16INK4a over adriamycin-sensitive controls (FIG. 48). It is apparent from the data that E-FABP, HnRNP and p16INK4a were effective marker genes for determining that a particular cell line was likely adriamycin-resistant.

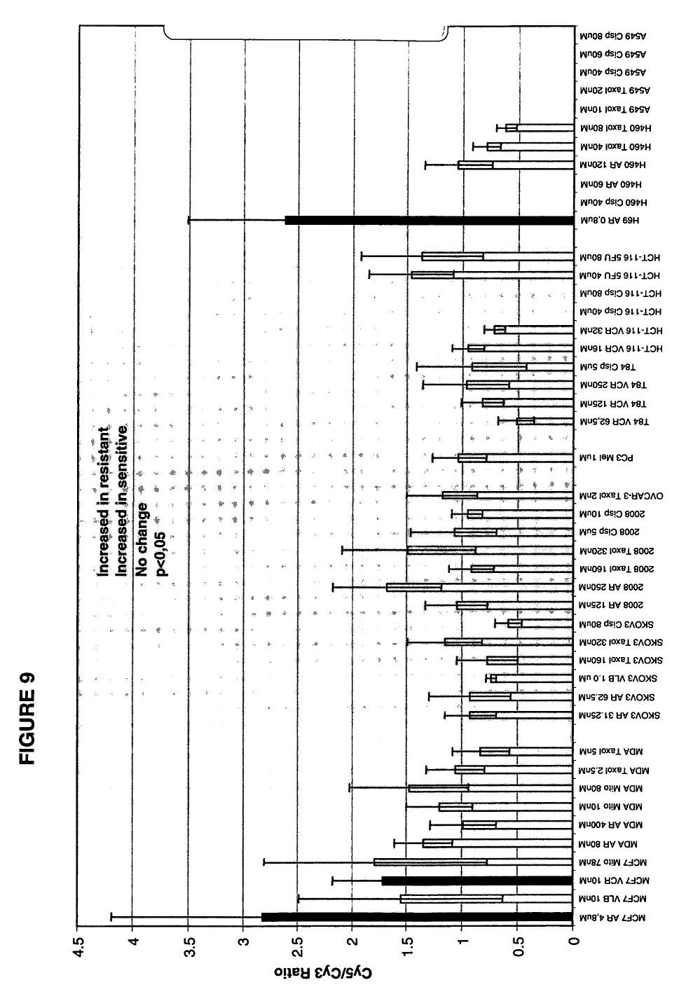

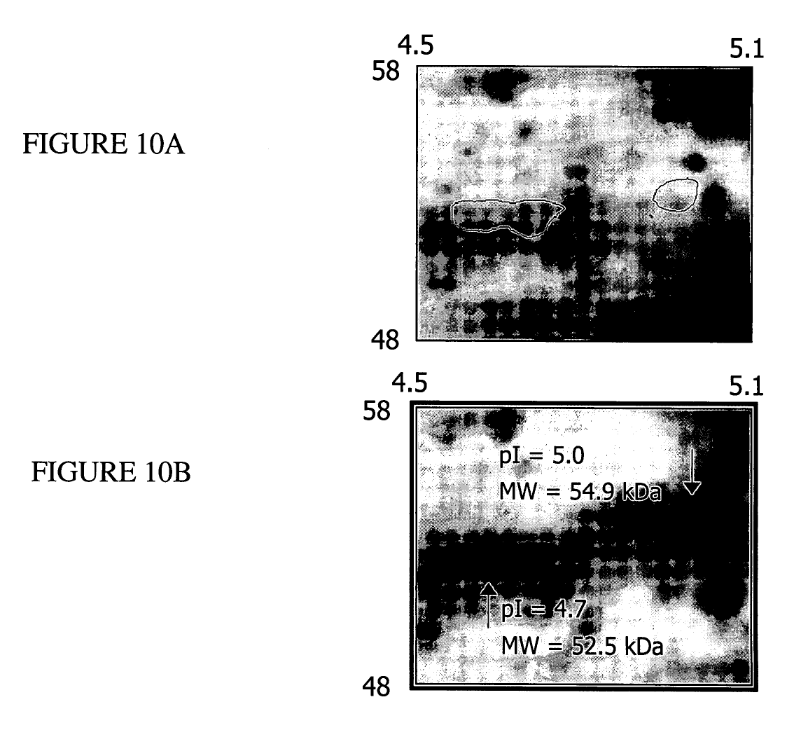

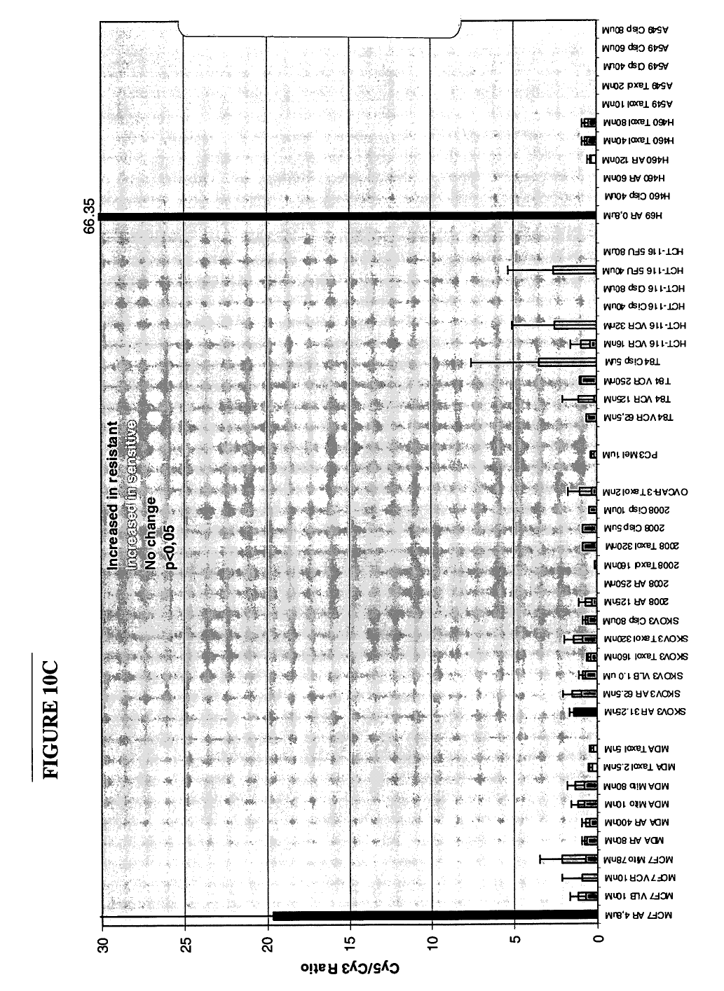

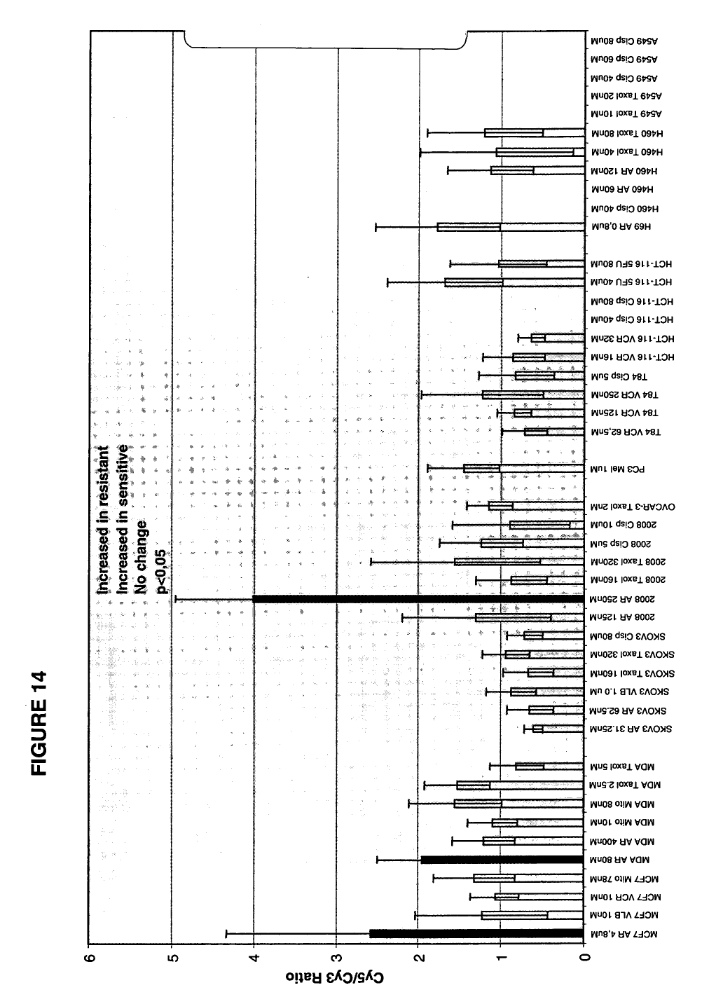

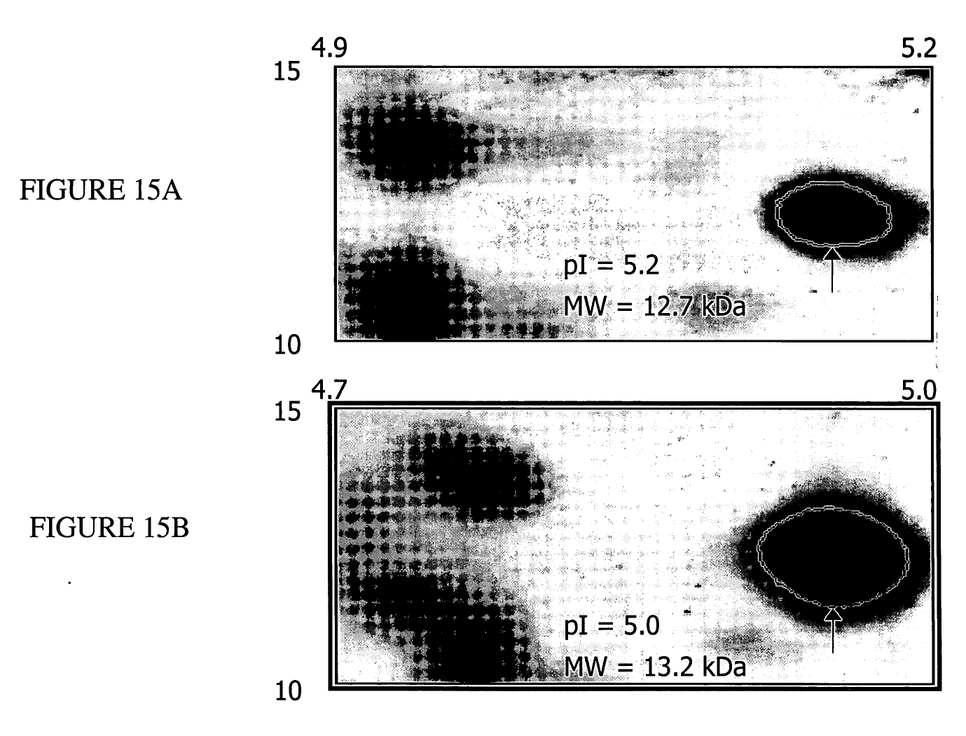

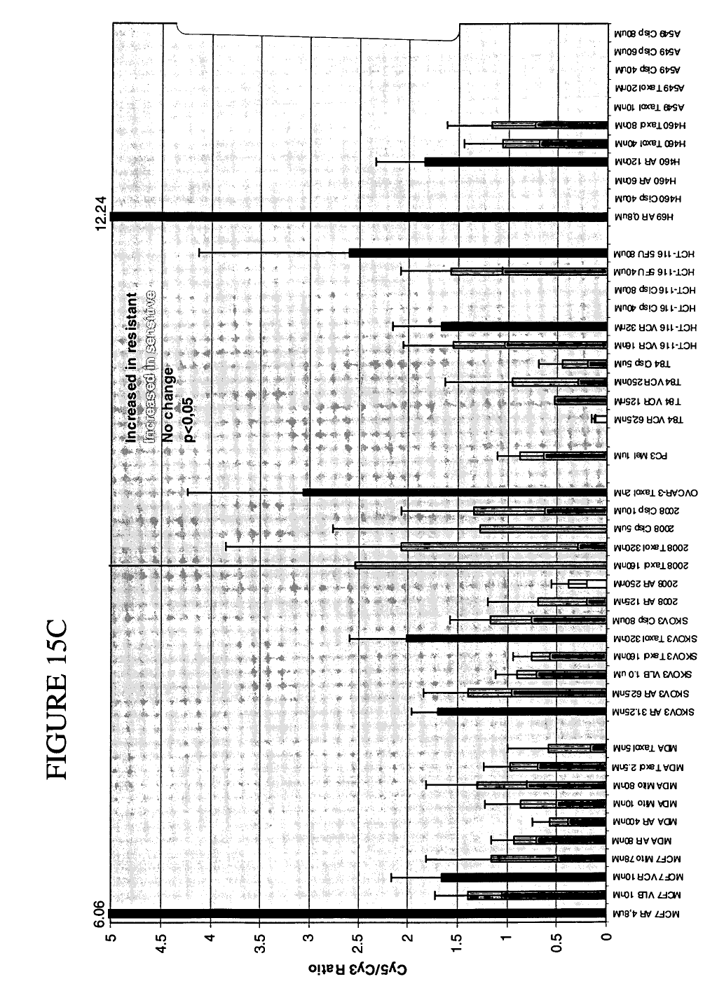

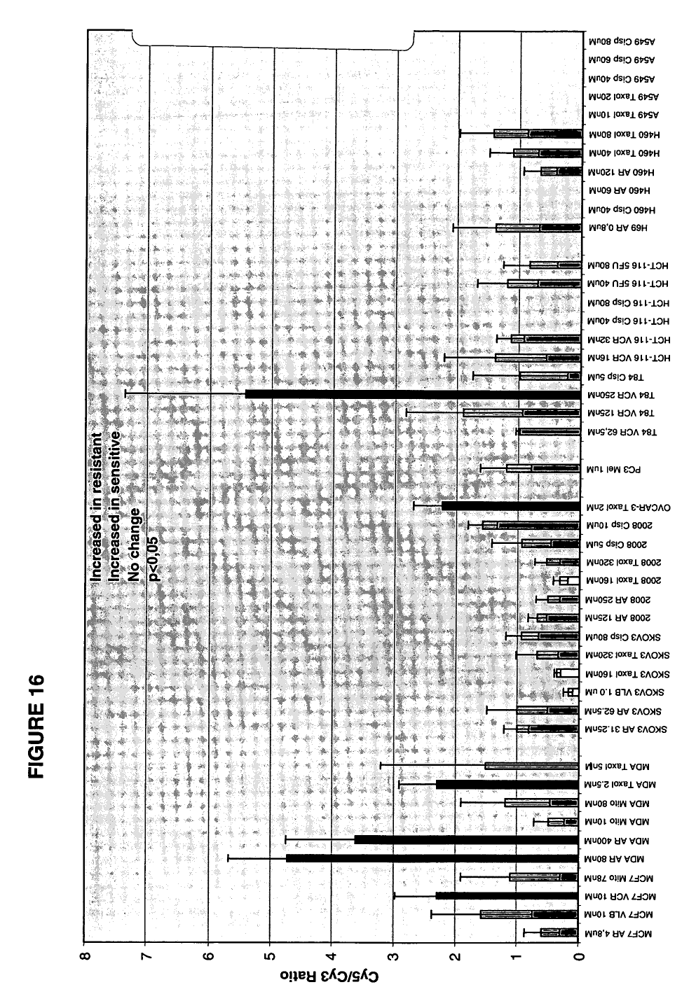

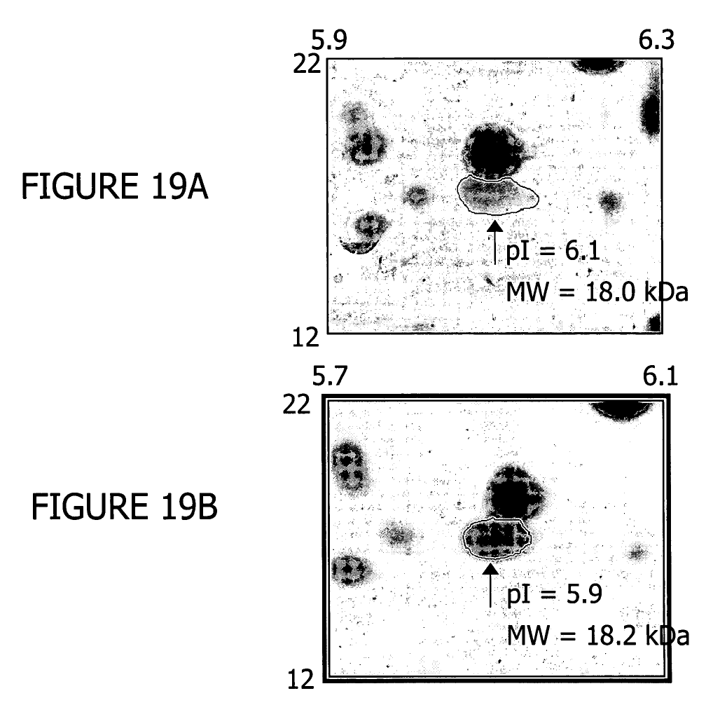

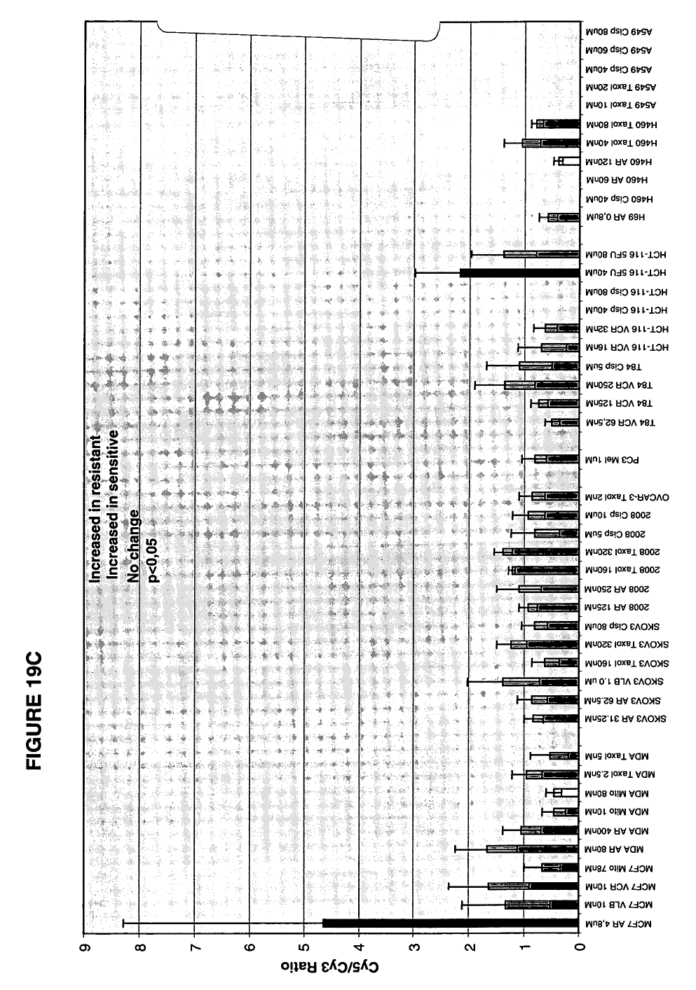

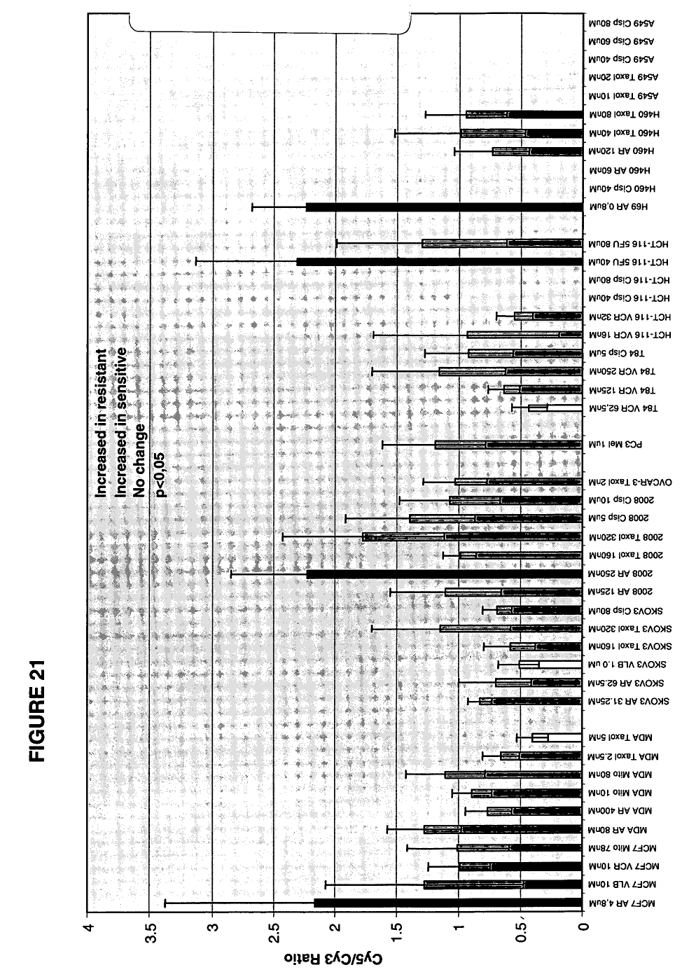

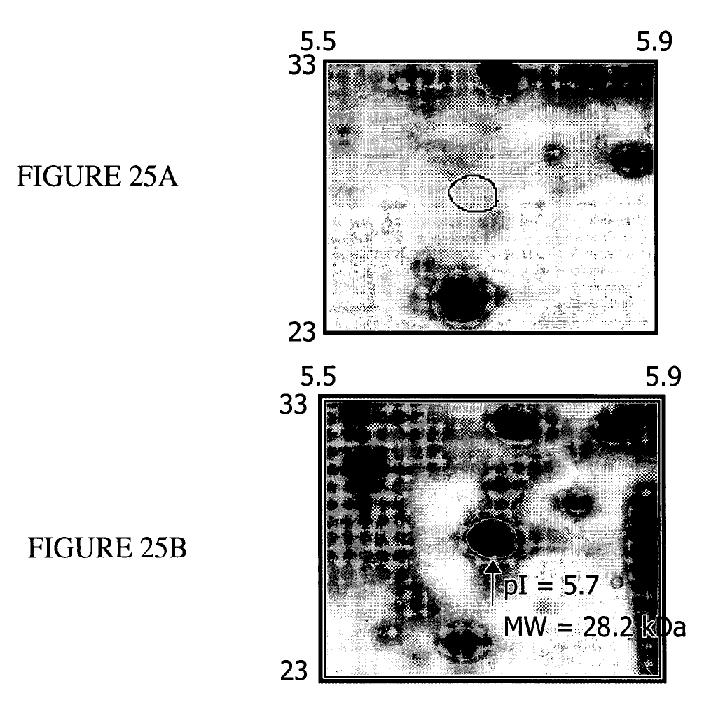

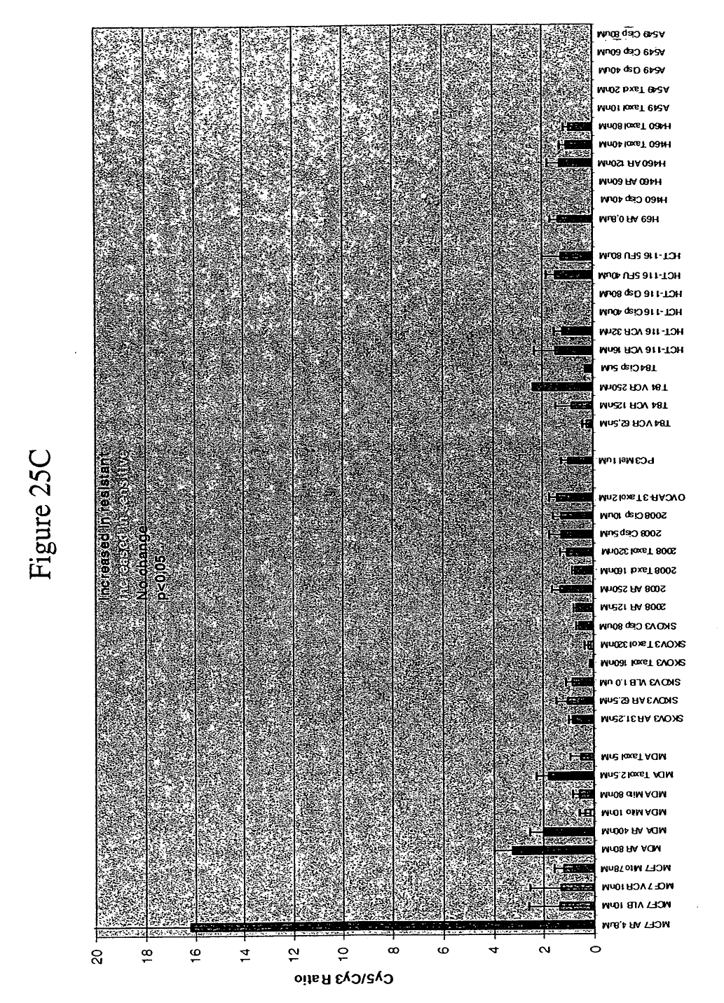

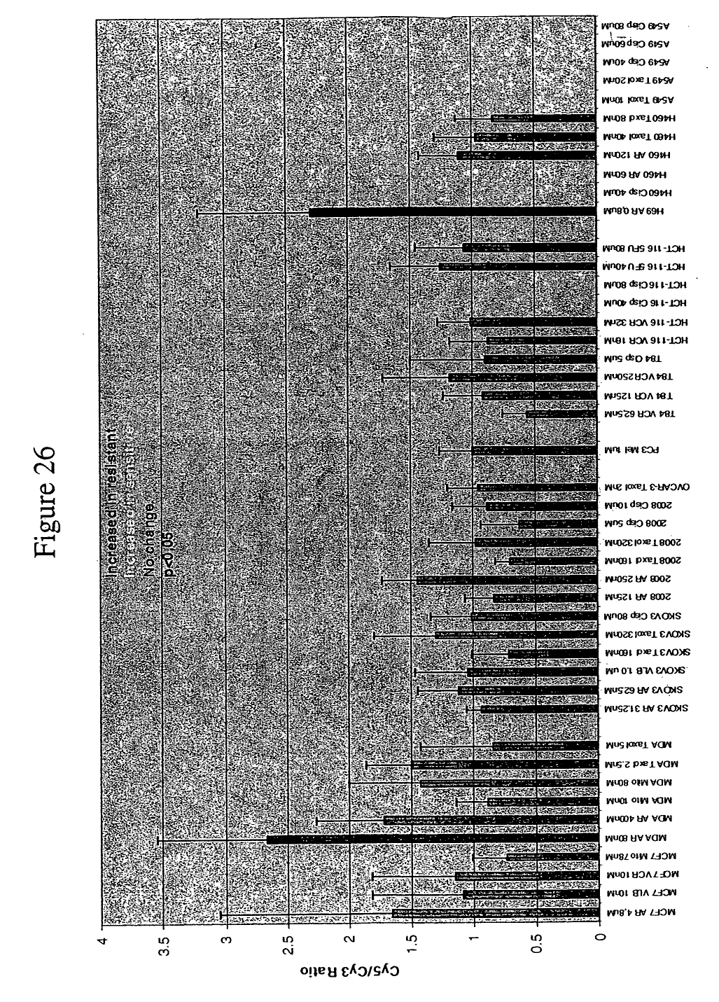

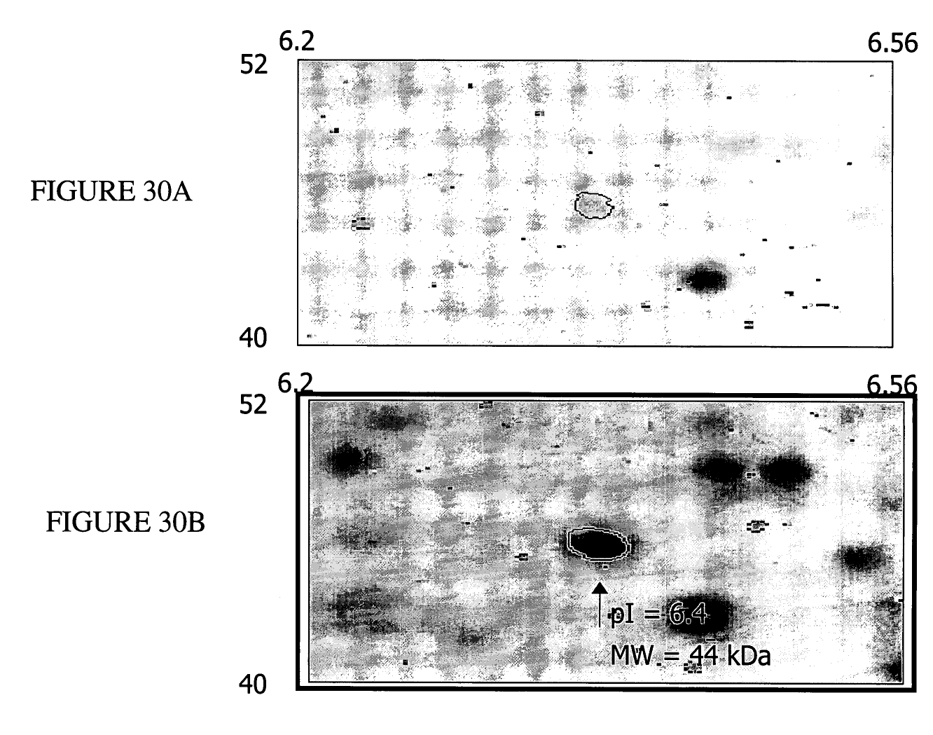

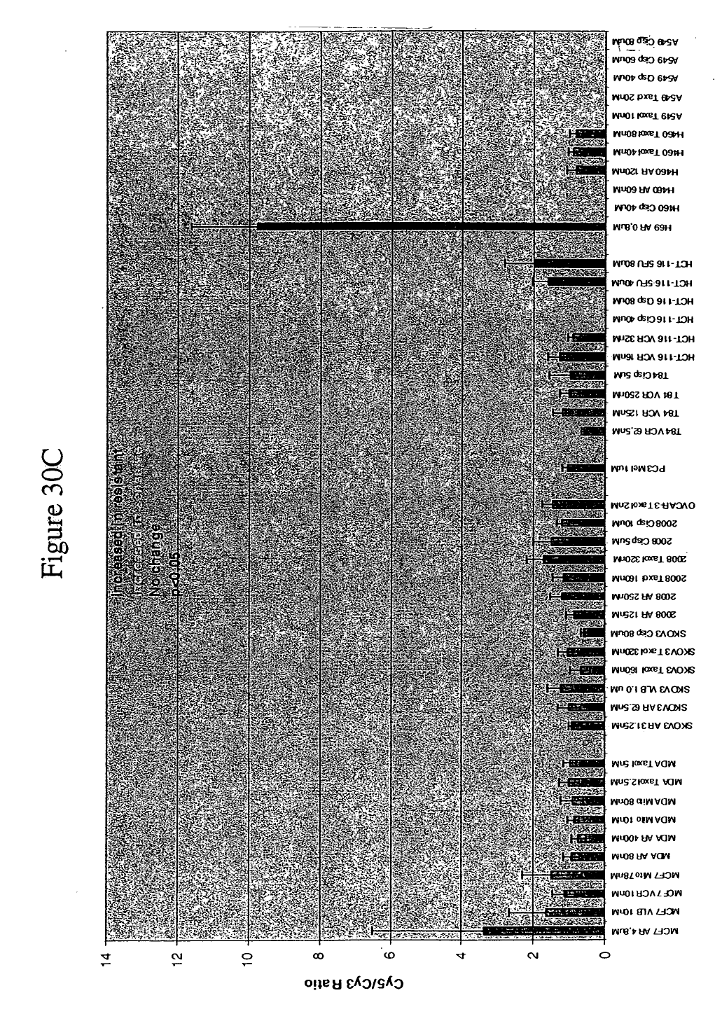

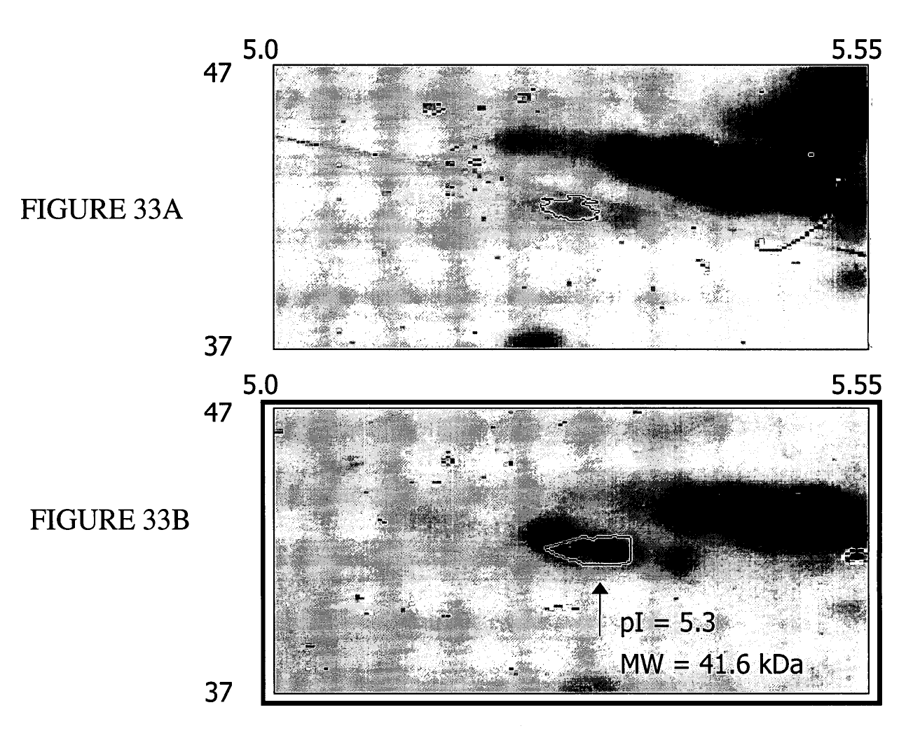

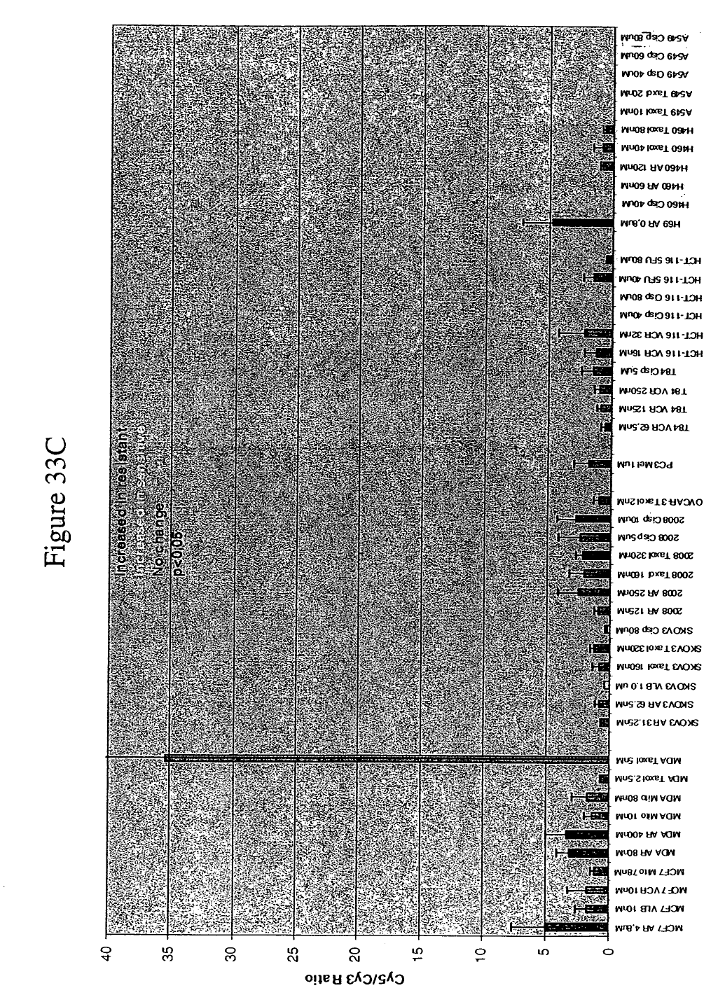

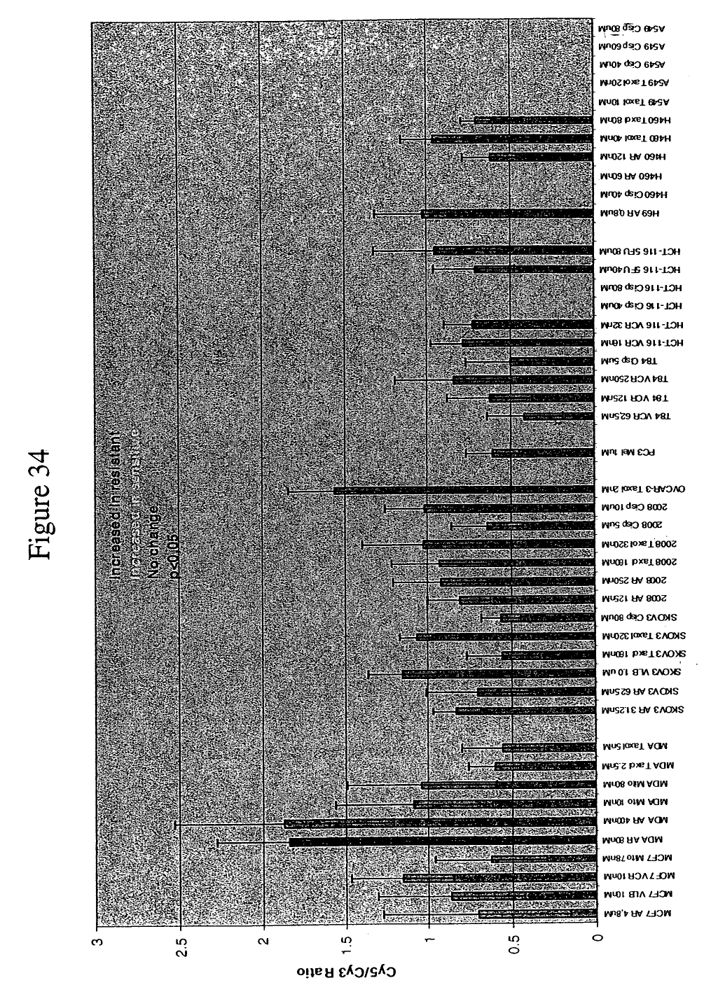

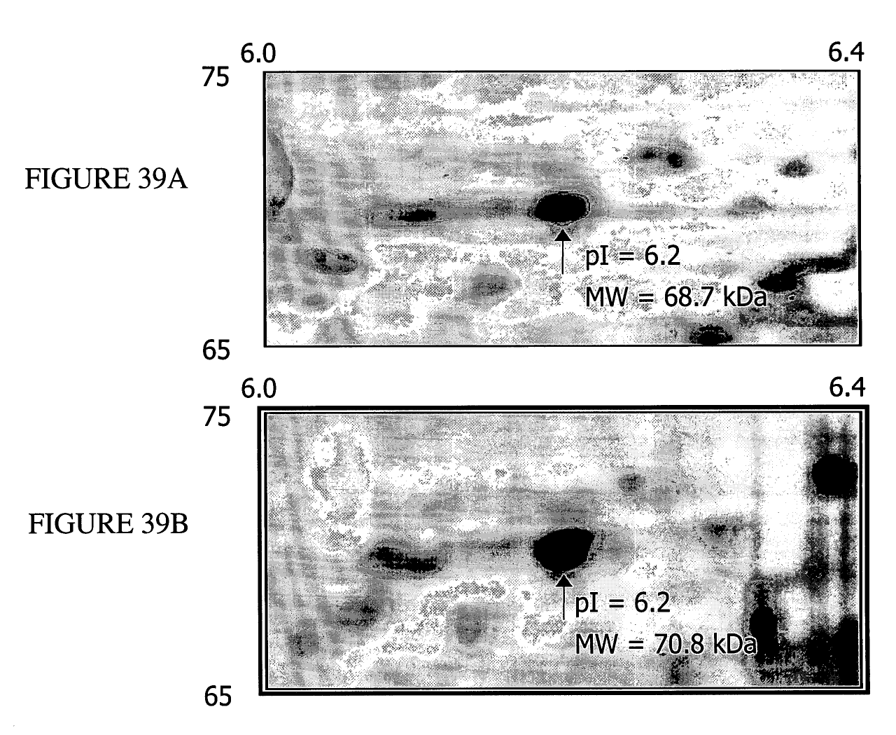

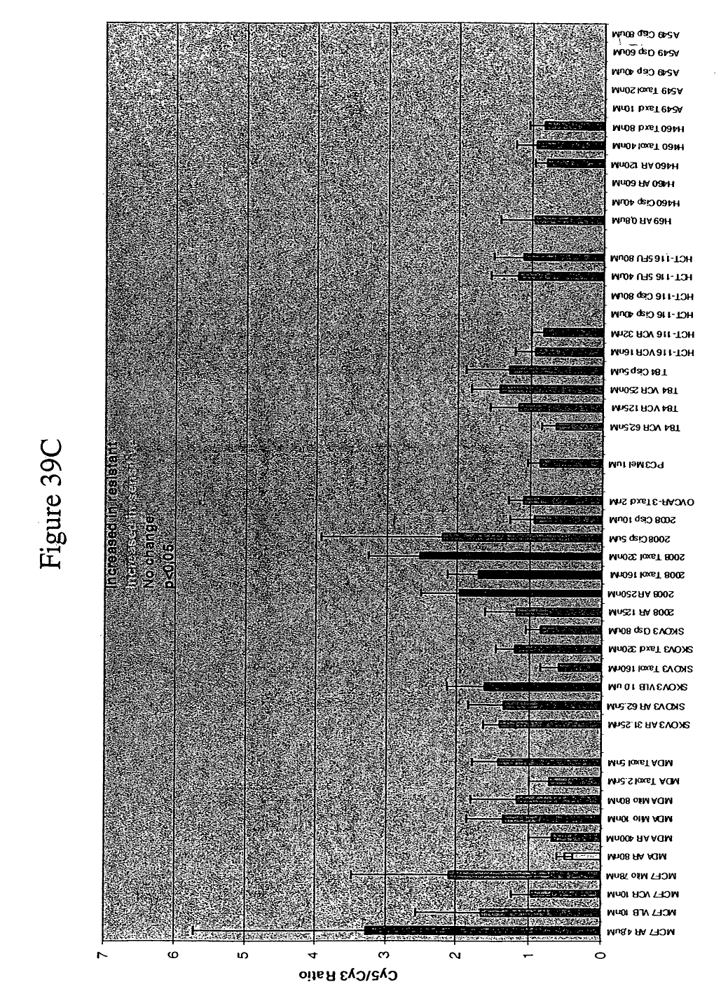

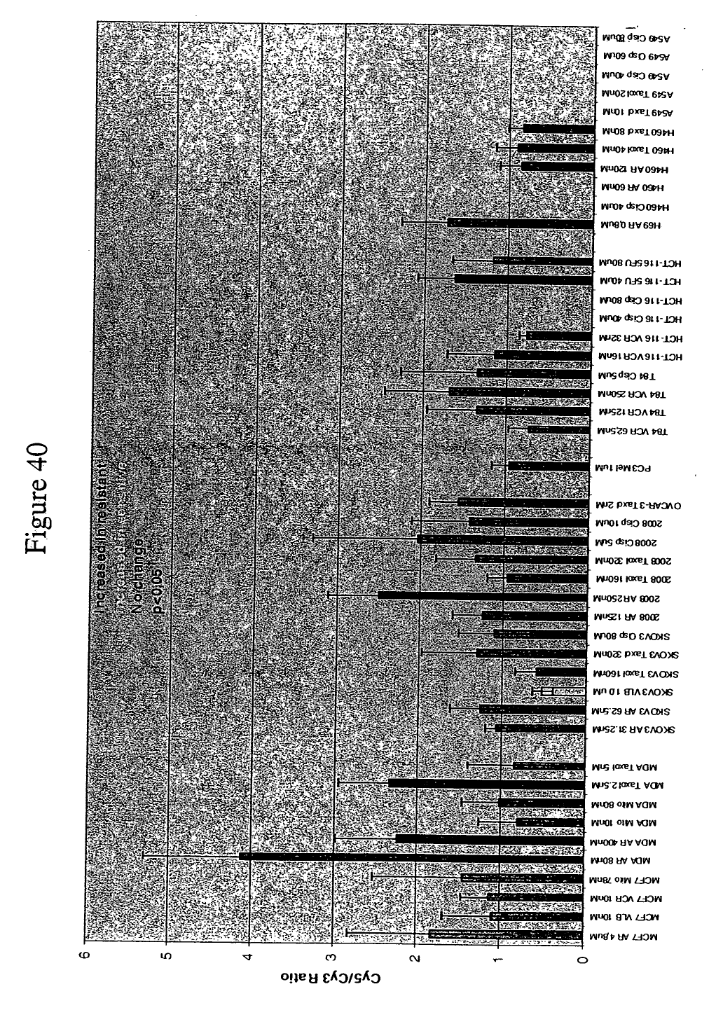

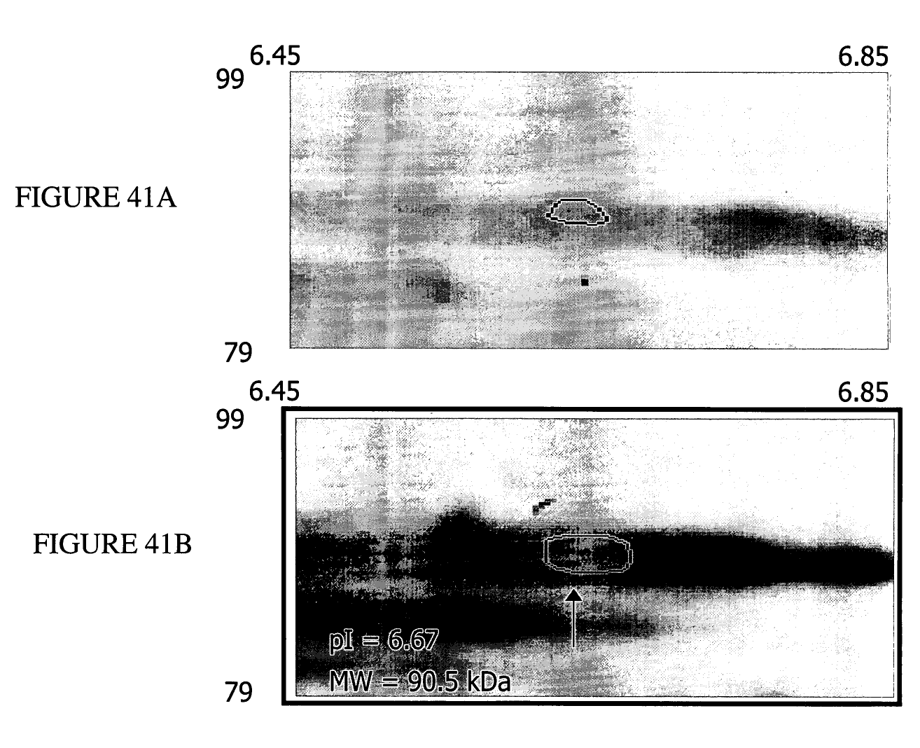

Microarray studies of adriamycin-resistant breast cancer cell samples also found that marker genes γ-actin (FIG. 9), vimentin (FIG. 10C), HSC70 (FIG. 14), galectin-1 (FIG. 15C), prosolin (FIG. 19C), β-tubulin (FIG. 21), GST-π (FIG. 25C), α-enolase (FIG. 30C), HSP27 (FIG. 16), tropomyosin 2 (FIG. 33C), PDI/ER-60 precursor (FIG. 39C), and SOD (FIG. 40) showed increased expression in adriamycin-resistant cell samples. Interestingly, marker genes γ-actin, vimentin, HSC70, galectin-1, prosolin, β-tubulin, GST-π, tropomyosin 2, and α-enolase were found in the MCF7 adriamycin-resistant cell sample, while marker genes HSP27 and SOD were found in the MDA cell sample. These results indicate that the microarray is capable of detecting differentially increased expression levels of mRNA between individual breast cancer cells.

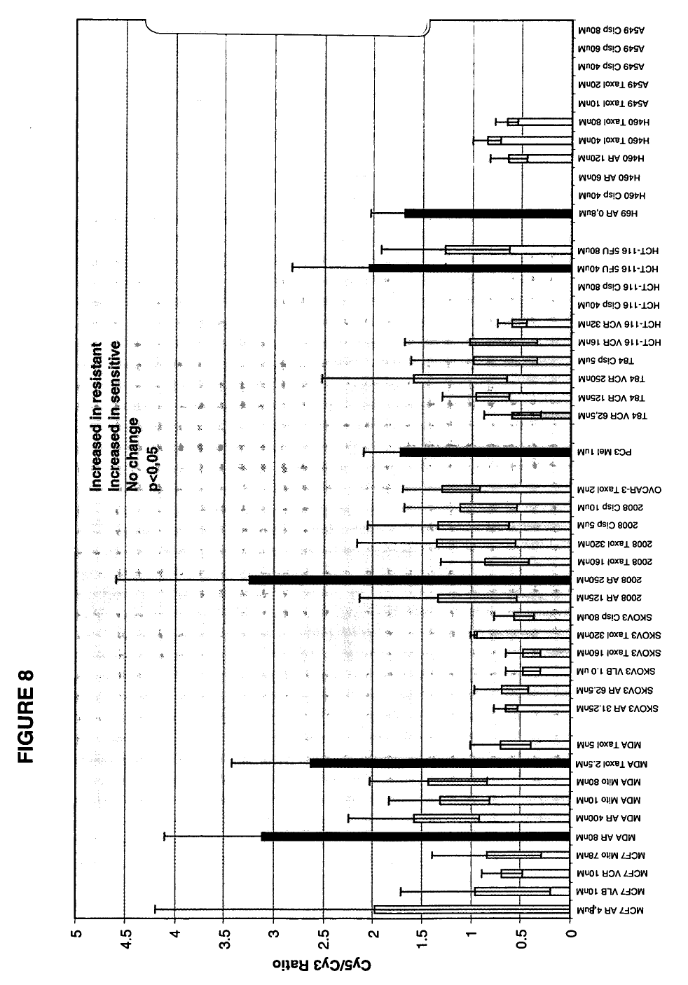

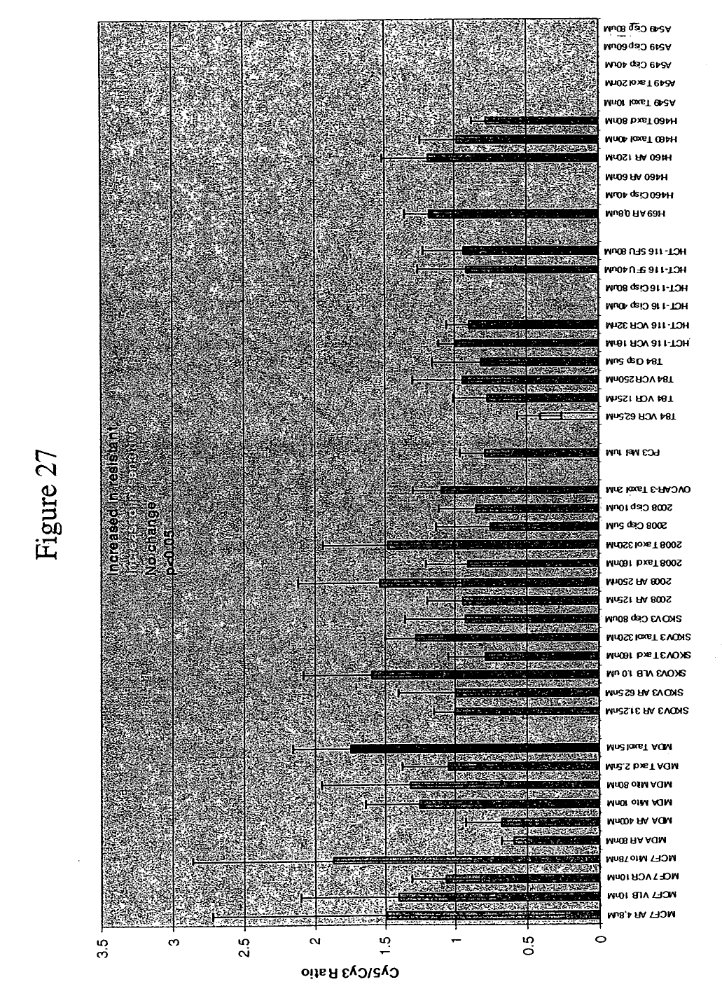

Labeled cDNA from a MDA taxol-resistant cell line was mixed with labeled cDNA from a MDA taxol-sensitive cell line for comparison of expression levels of marker genes in the drug-resistant cell line against the expression levels of marker genes in the drug-sensitive cell lines. As with adriamycin-resistant breast cancer cell lines, annexin-1 showed increased expression in the taxol-resistant cell lines (FIG. 12). Also, the marker genes GST-π (FIG. 25C), HSP27 (FIG. 16), and SOD (FIG. 40) were increased in taxol-resistant MDA cells when compared to control taxol-sensitive MDA cell lines. Additional marker genes such as PDI and HSP60 also showed statistically significant increases in expression levels in taxol-resistant cell samples (FIGS. 27 and 8). These results indicate that markers such as annexin-1 are indicators for drug resistance to multiple types of chemotherapeutic drugs. These results also indicate that marker gene profiles differ between cells resistant to different chemotherapeutic drugs.

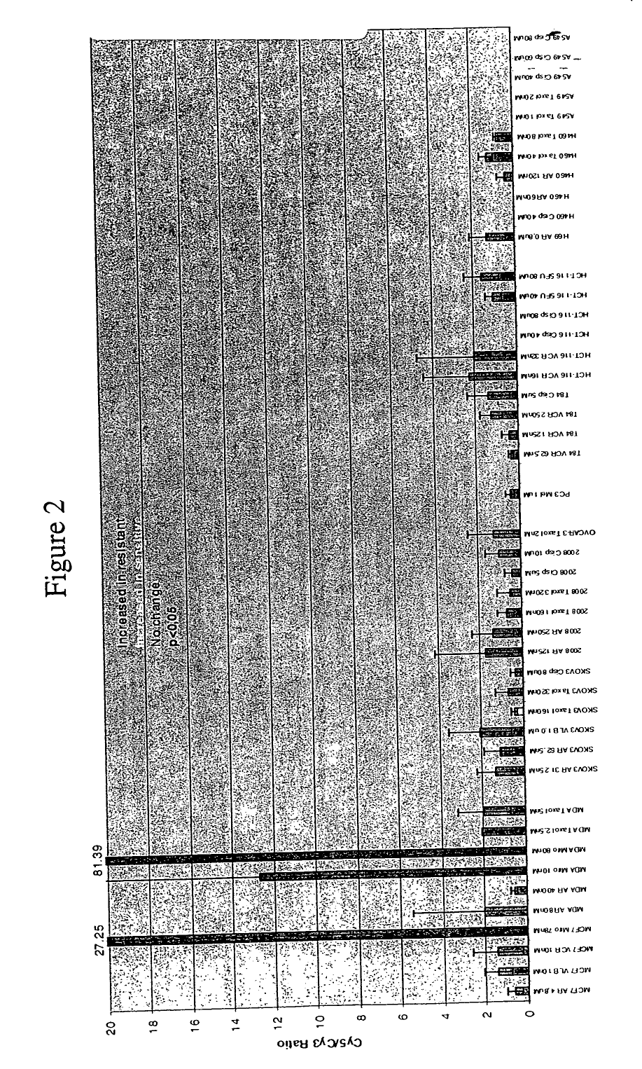

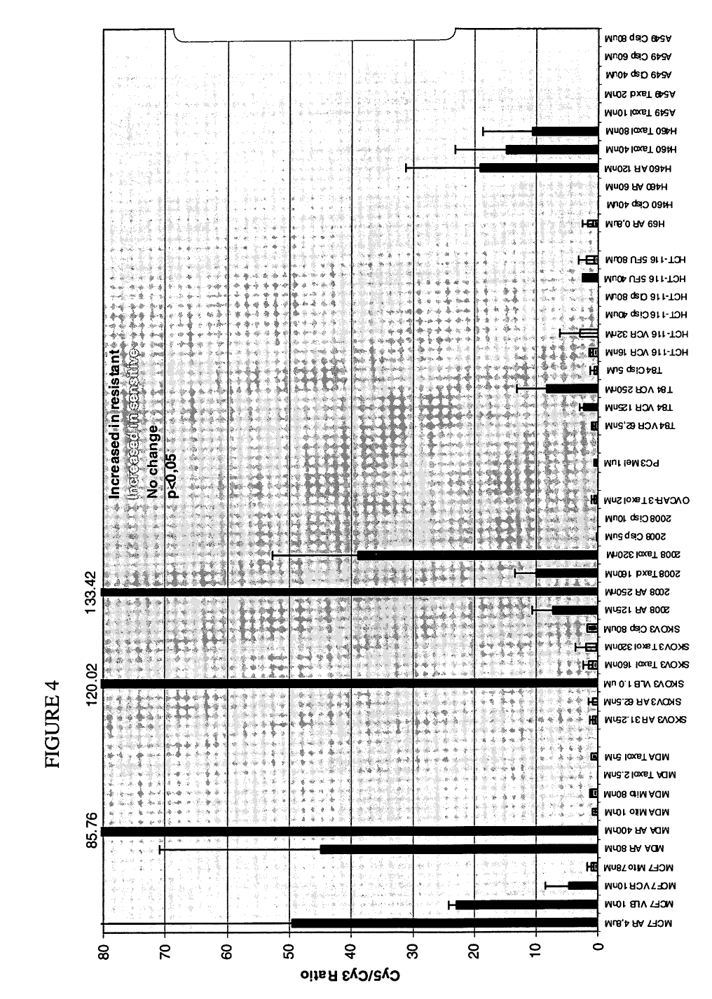

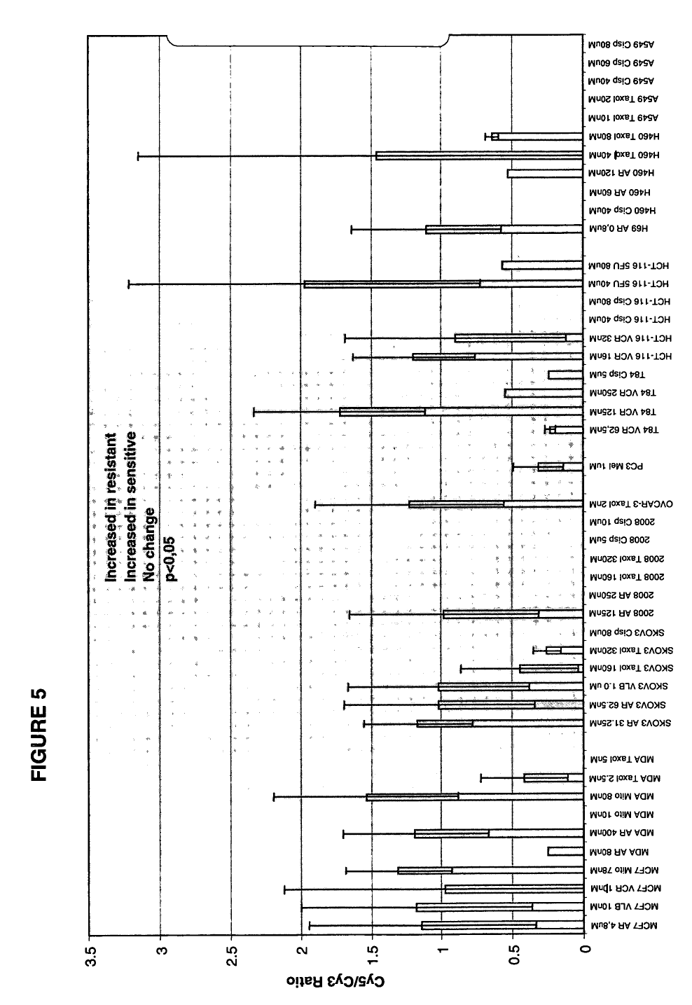

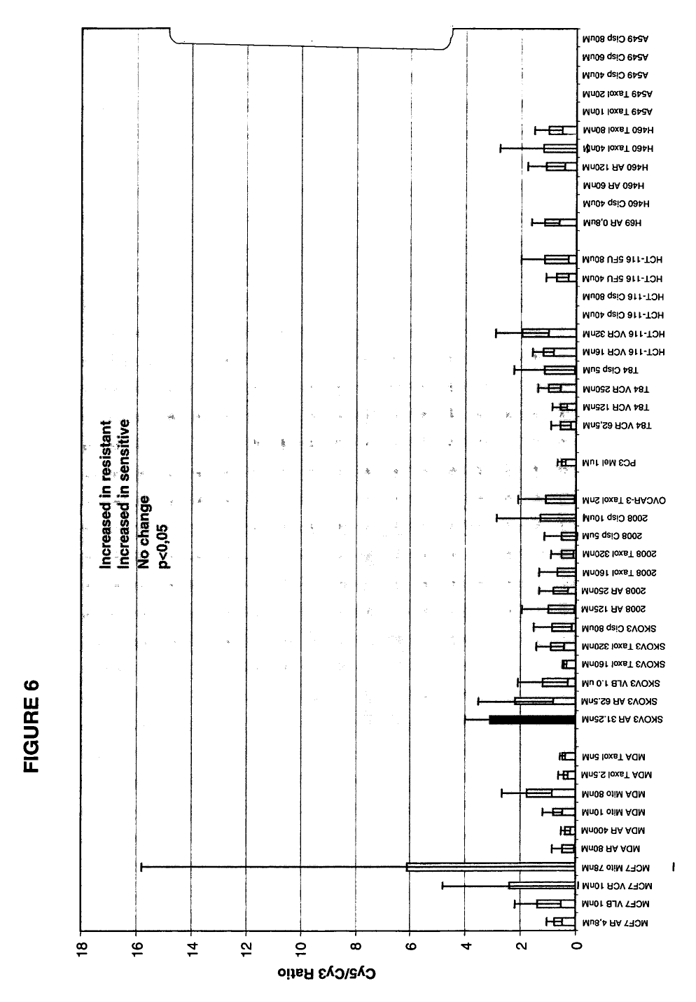

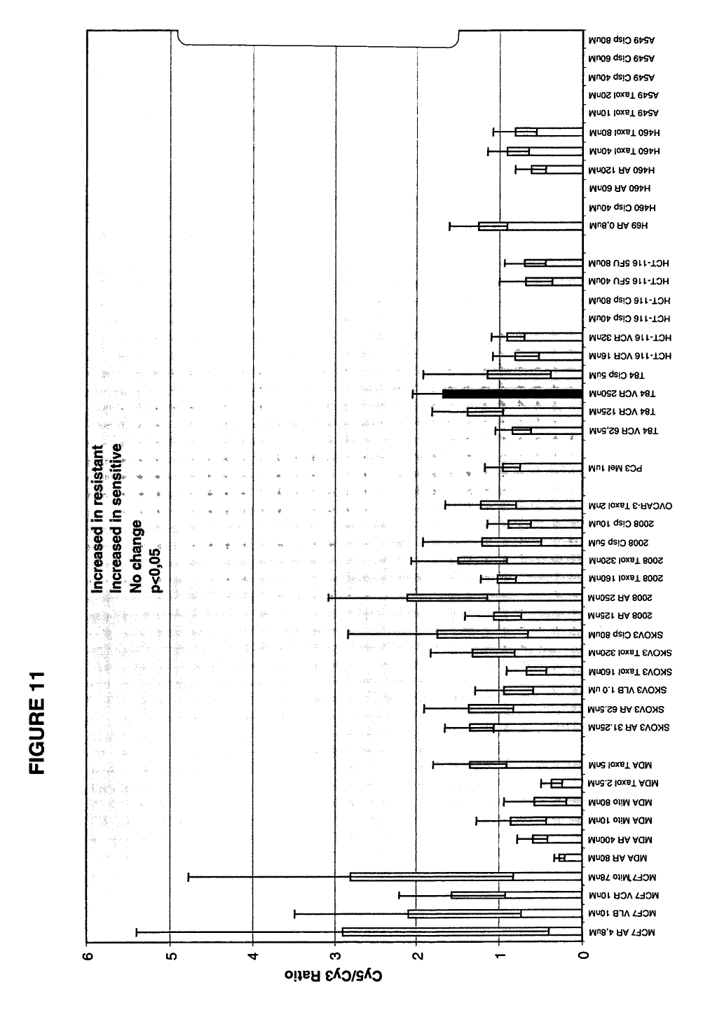

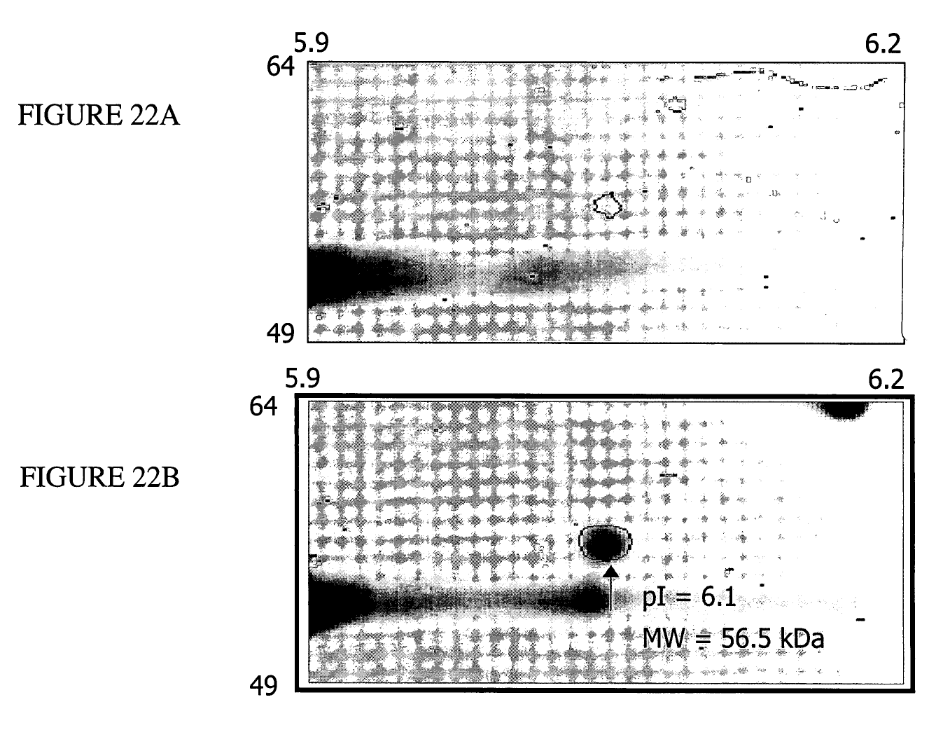

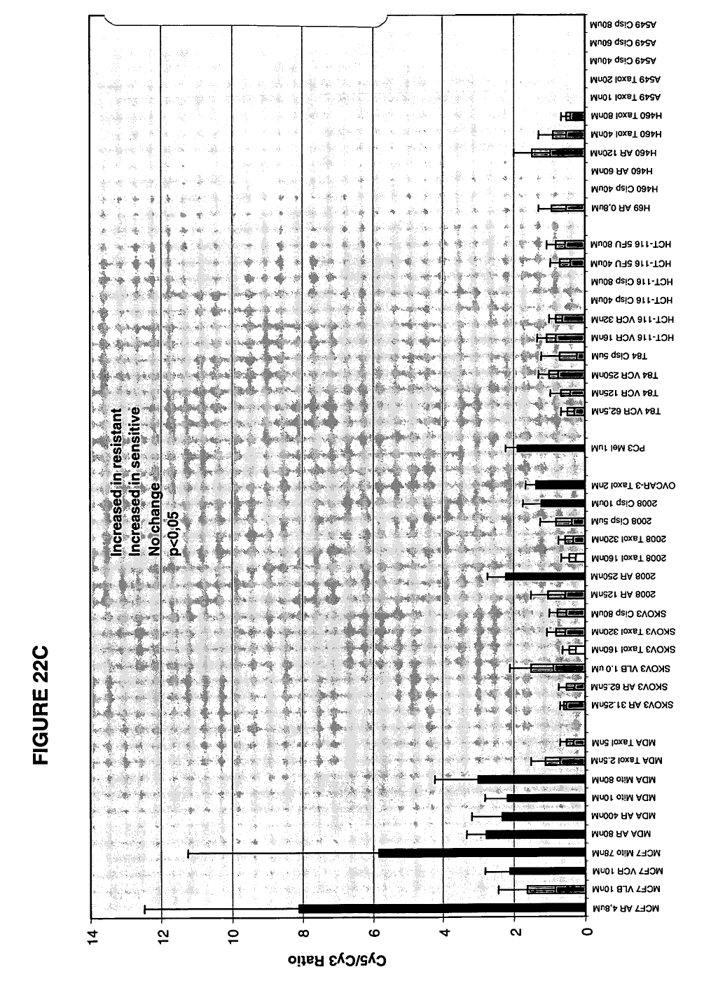

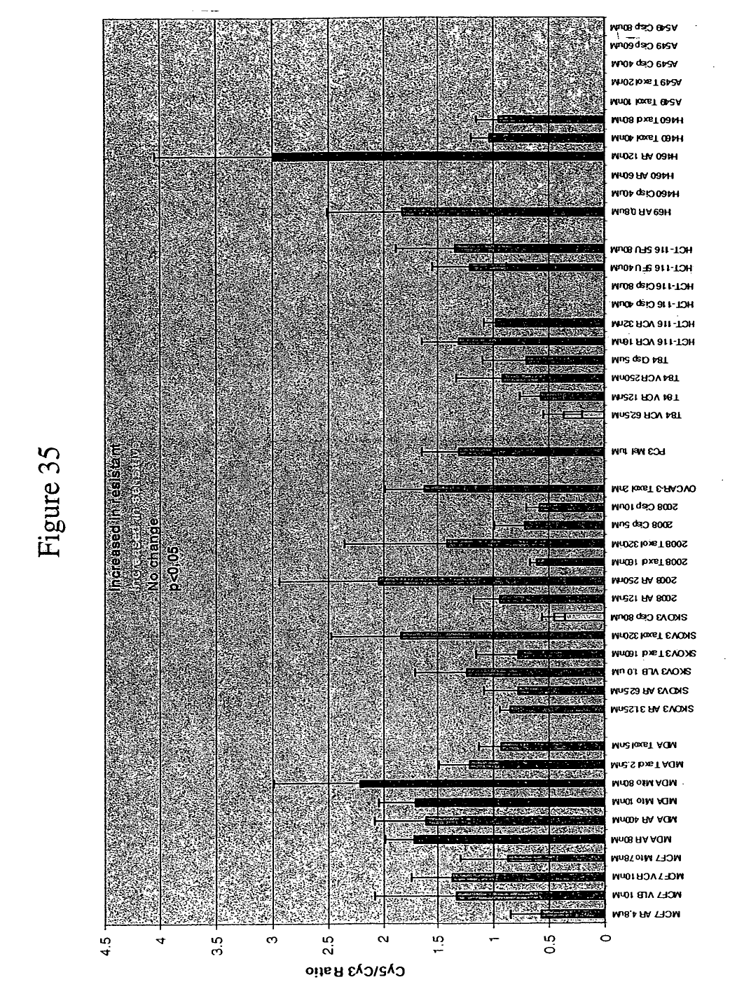

The focused microarray was tested for its capacity to identify marker genes with increased expression in vincristine-resistant MCF7 cell lines. The marker genes γ-actin (FIG. 9), HSP27 (FIG. 16), Pgp 1 (FIG. 4), and ezrin (FIG. 22B) were identified as having increased levels of expression in vincristine-resistant cell lines when compared to vincristine-sensitive cell lines. The marker genes γ-actin and HSP27 is expressed at greater levels in multiple cell lines that are resistant to different drugs (FIGS. 9 and 16). In addition to vincristine resistance, breast cancer cell lines resistant to mitoxantron were studied using the focused microarray. The studies indicated that the marker genes ezrin (FIG. 22C), BIP (FIG. 11), BCRP (FIG. 2), and keratin type II (FIG. 35) showed increased expression in the mitoxantron resistant MDA and MCF7 cell lines compared to mitoxantron sensitive cell lines. The cell markers γ-actin and ezrin showed increased expression in both MDA and MCF7 cell lines, indicating that these marker genes are generally expressed in breast cancer cells resistant to mitoxantron. The focused microarray also identified differential expression of marker genes between breast cancer cell lines, which establishes the sensitivity of the device to find minor differences in marker gene expression between cells obtained from the same tissue. This finding shows that the focused microarray can identify increased expression of marker genes that are not expressed in all cells of the same tissue type, but still indicate that drug resistance exists in the individual cancer cell sample.

An MCF7 breast cancer cell line resistant to vinblastin was also studied to determine the marker genes that were increased in expression in resistant breast cancer cell samples. The Pgp 1 marker gene was identified by a comparison of expression levels in a vinblastin-resistant cell sample to the expression levels in a vinblastin-sensitive cell sample (FIG. 4). The focused microarray has identified increased expression of Pgp 1 in cell lines resistant to vincristine, vinblastin, taxol and adriamycin. The cell lines were also derived from breast, ovarian, colon, and lung tissues.

To further elucidate the ability of the focused microarray to ascertain differences in marker gene expression between cancer cell samples, ovarian cancer cell lines were obtained for screening studies using the focused microarray (see Table 1). The focused microarray was hybridized with a mixture of labeled-cDNA produced from cell sample RNA obtained from the drug-resistant breast cancer cell lines and the drug-sensitive breast cancer cell lines. The hybridization of a mixture of cDNA represented the comparison of expression between the drug-sensitive cell sample and the drug-resistant cell sample. The cDNA was labeled with the Cy5/Cy3 fluorescent dye system. Hybridization of labeled cDNA to capture probes was analyzed as described for the breast cancer cell line samples.

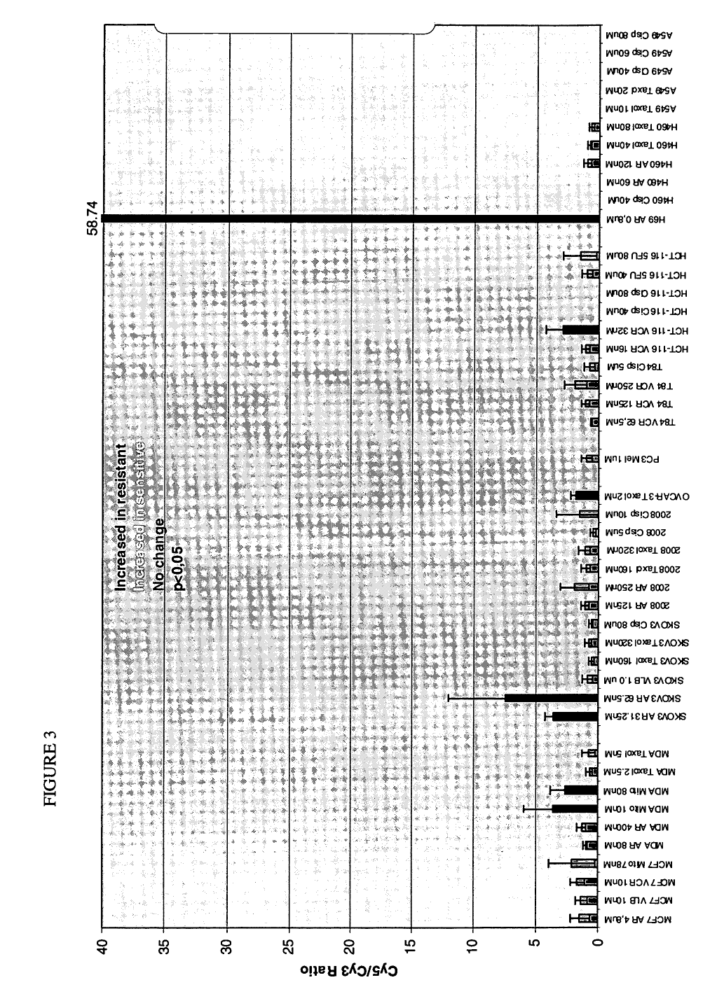

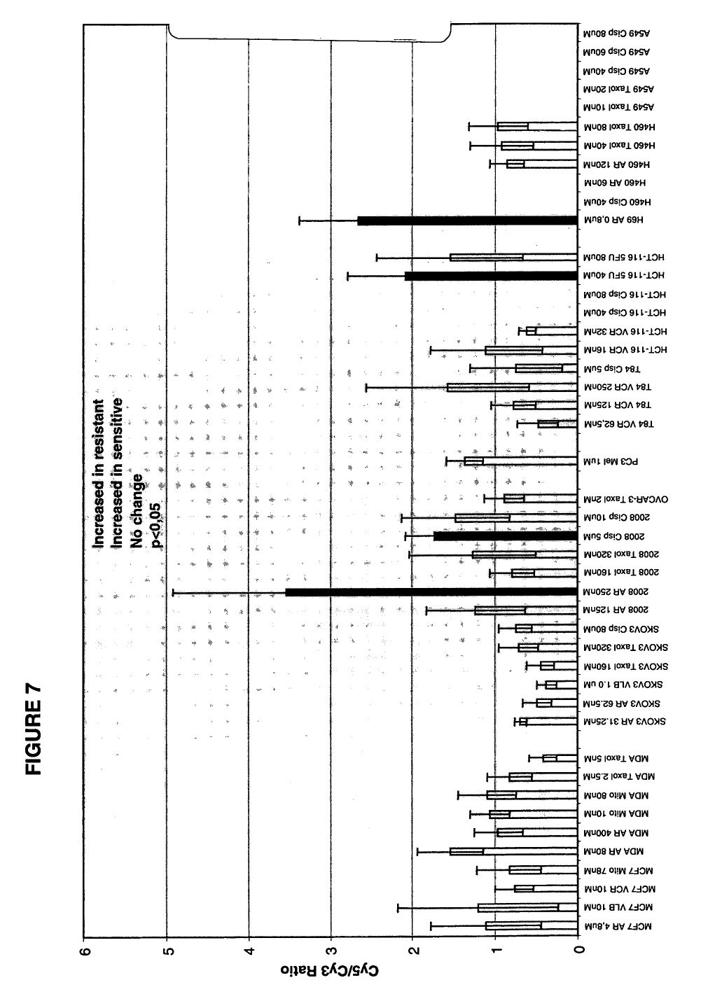

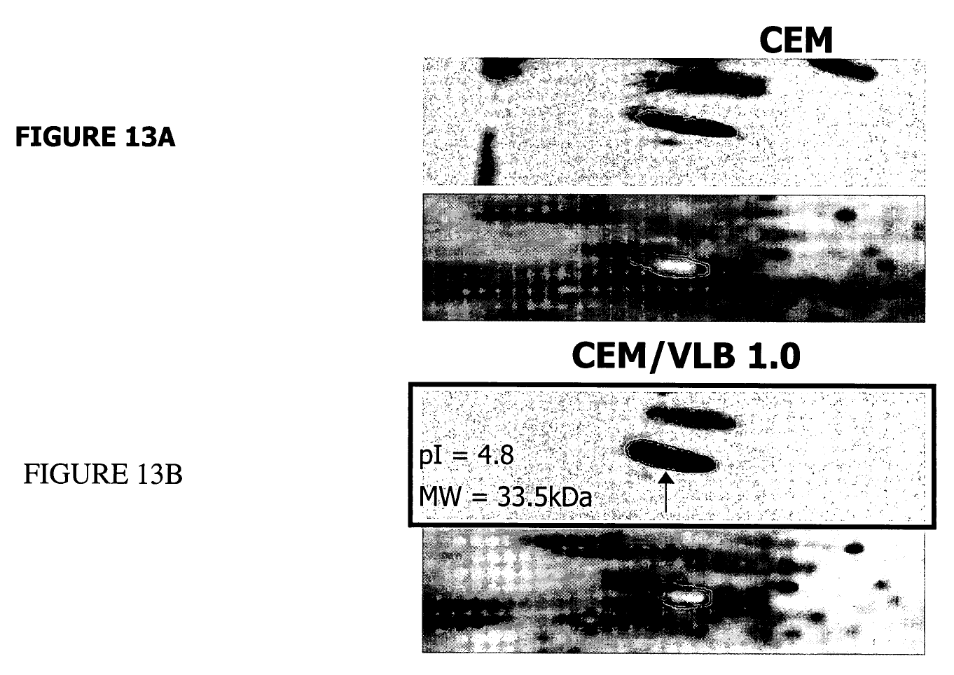

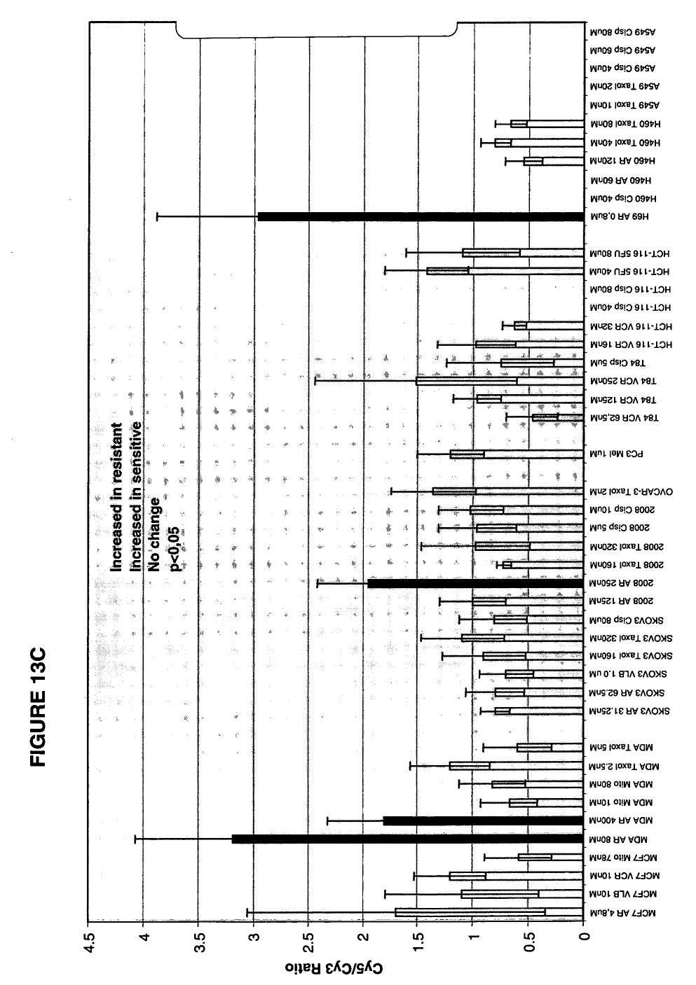

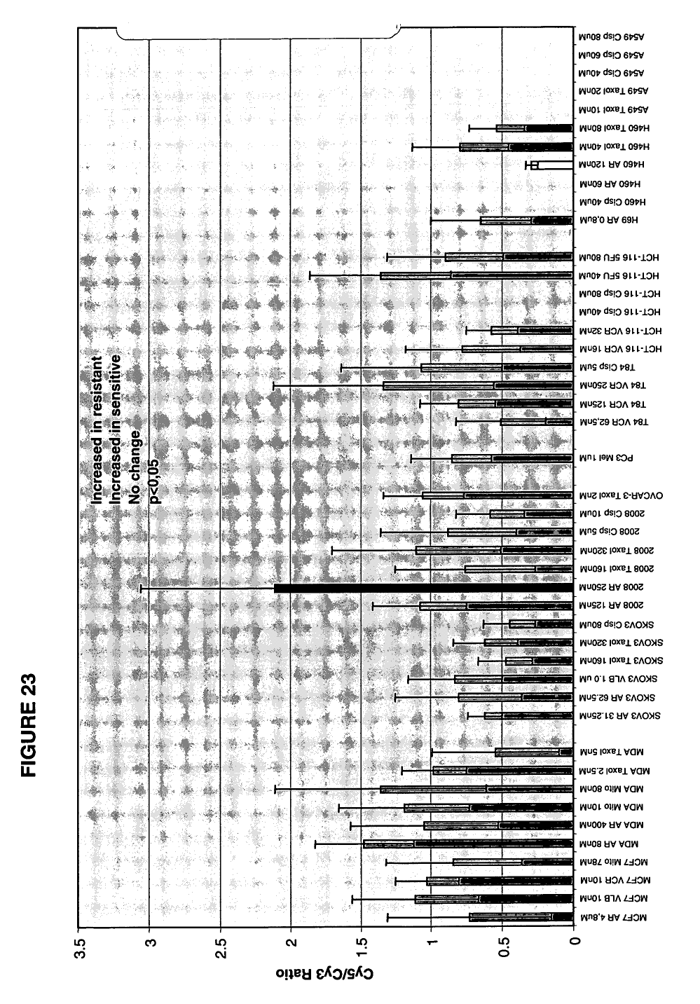

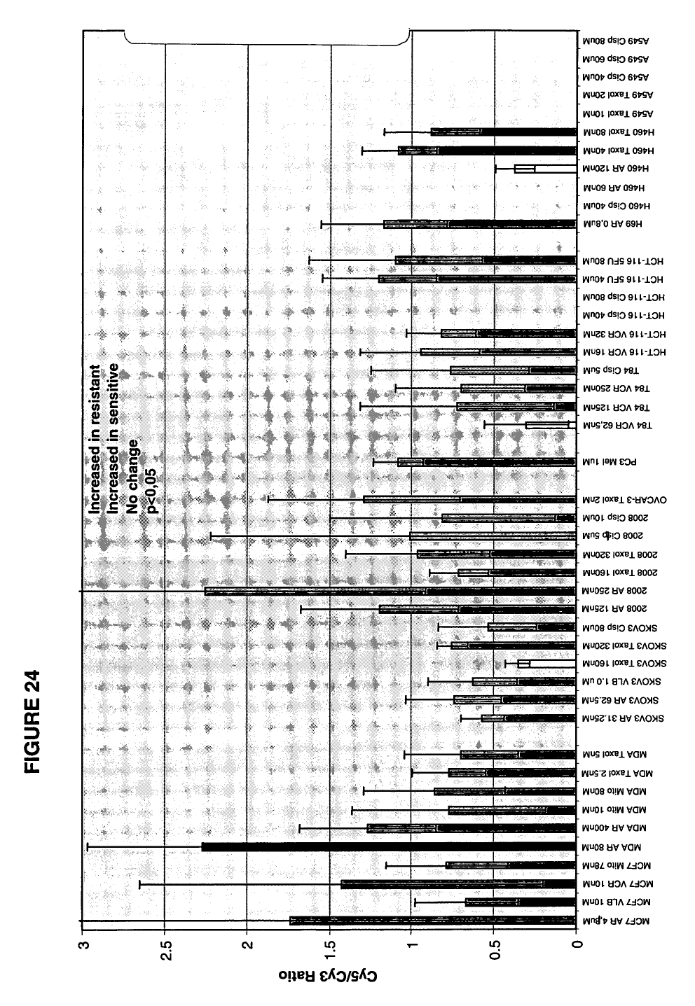

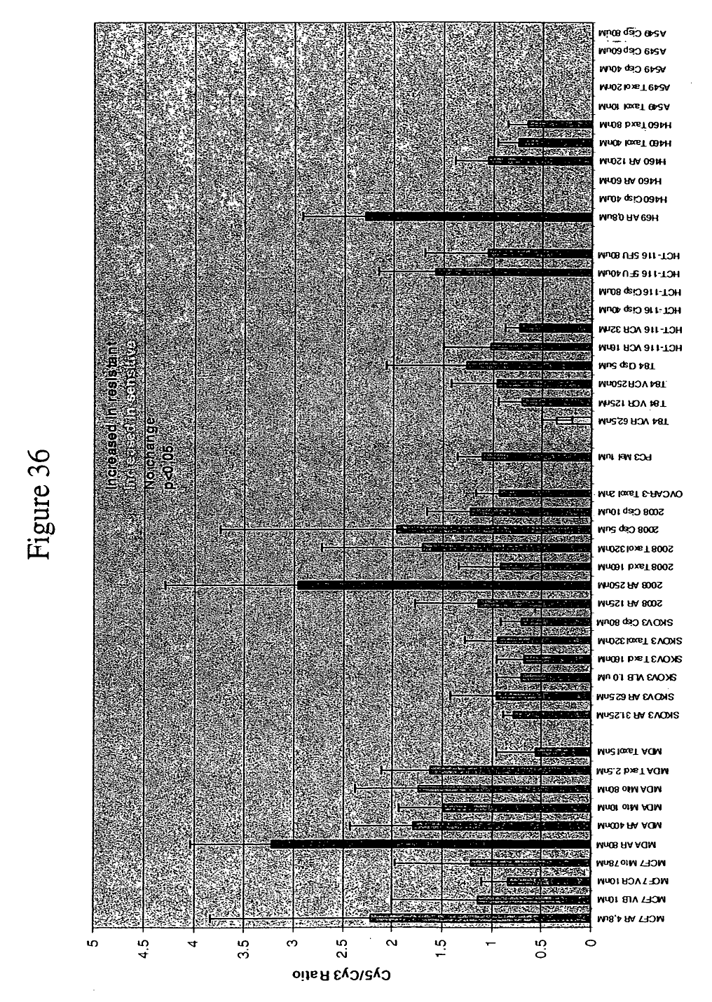

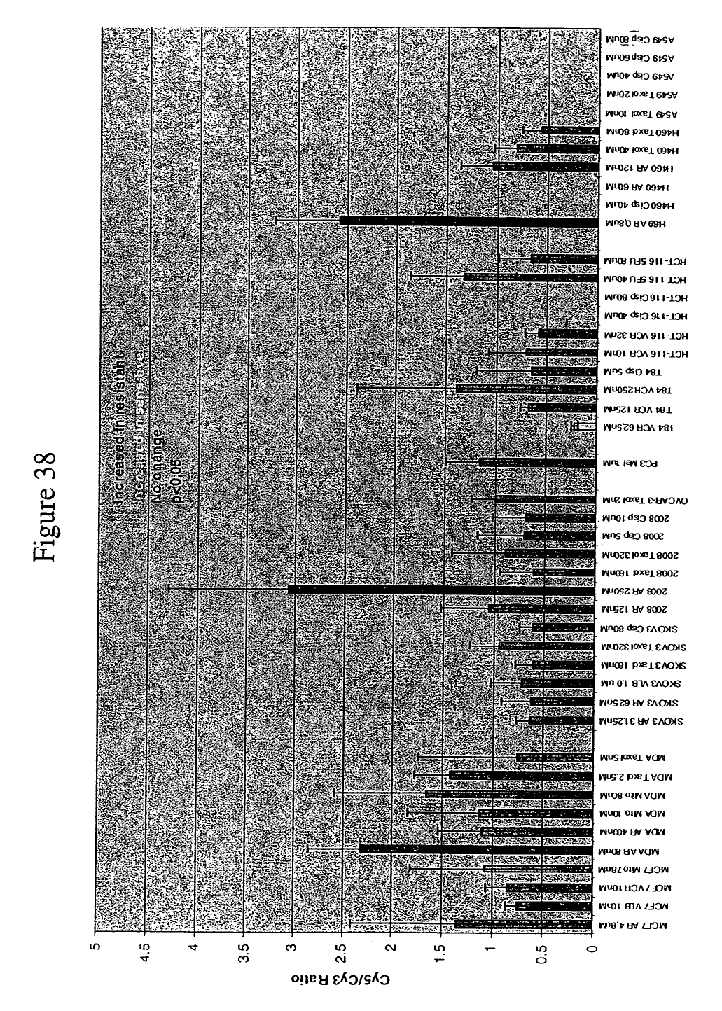

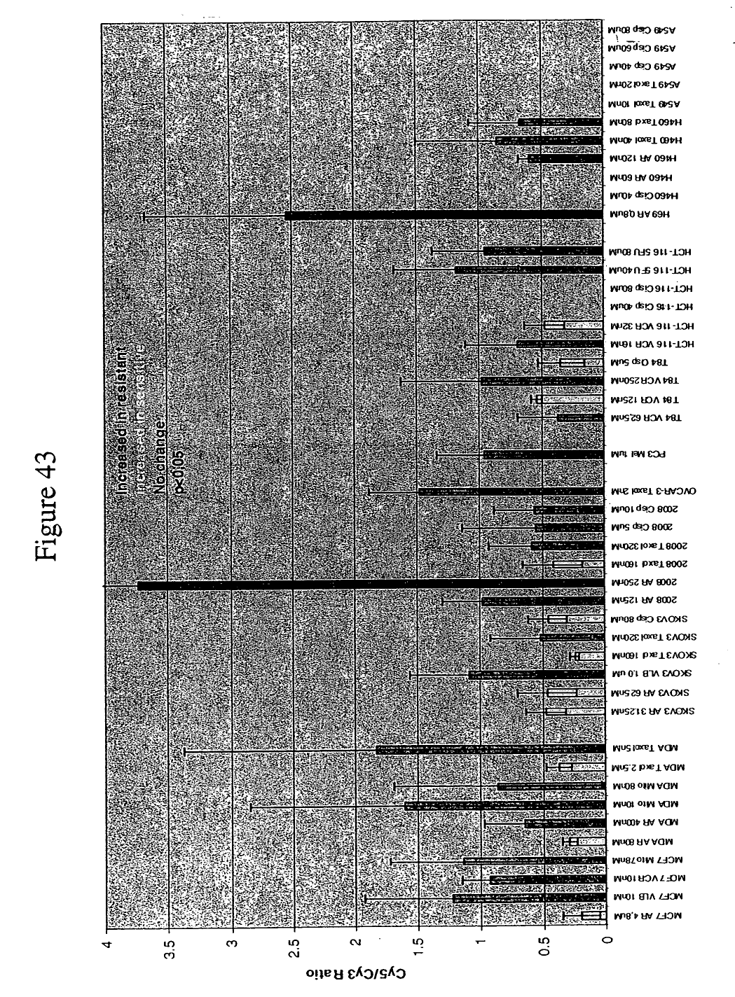

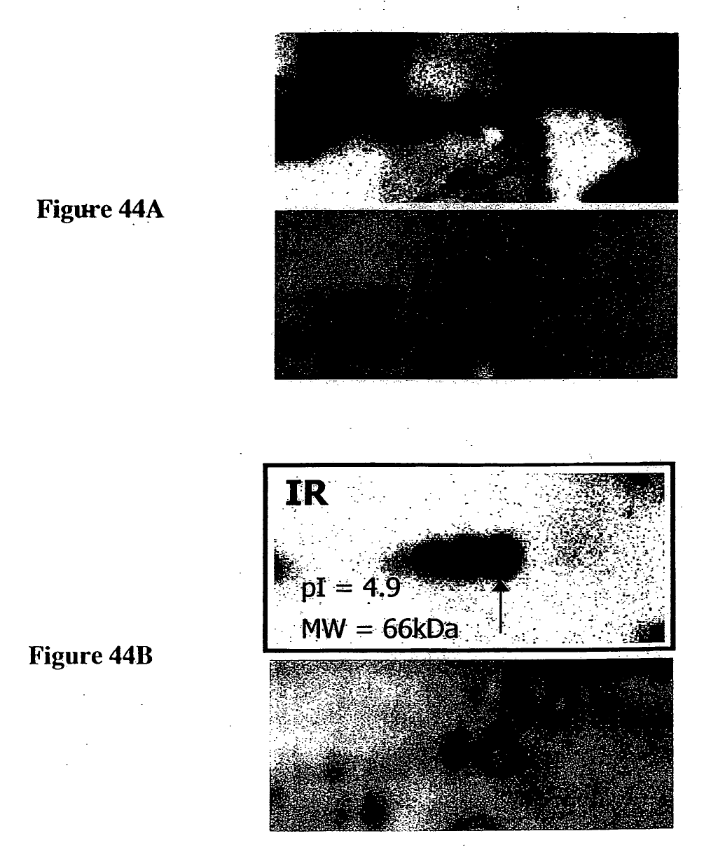

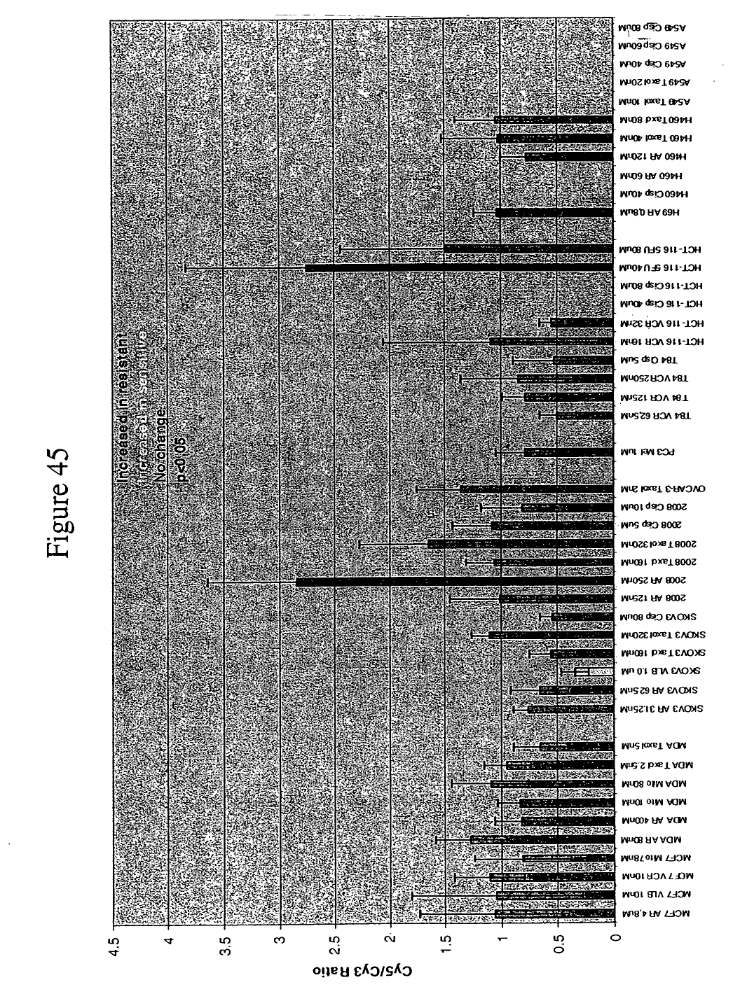

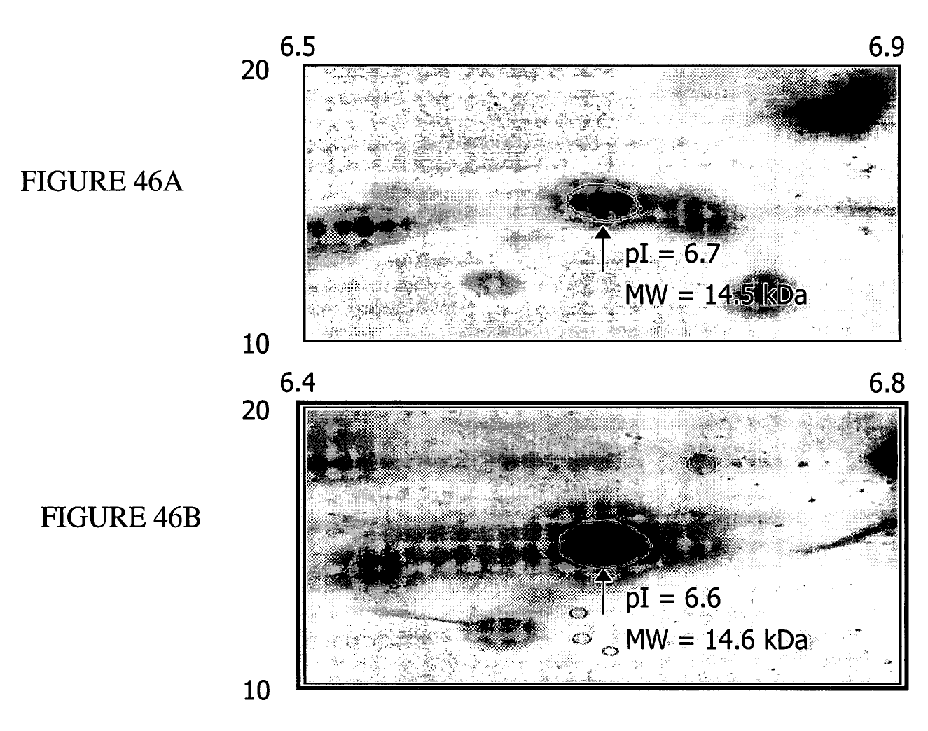

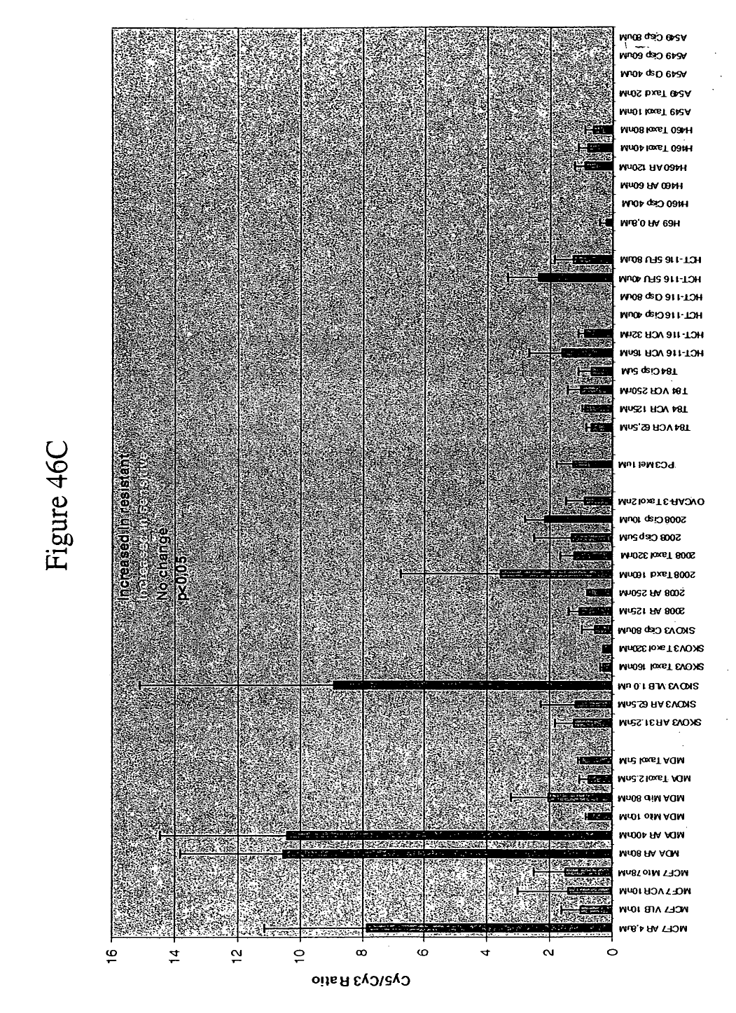

The study used ovarian cancer cell lines obtained from epithelial adenocarcinomas. The adriamycin-resistant 2008 and SKOV3 ovarian cancer cell line samples showed increased expression in annexin-1 (FIG. 12), HSC70 (FIG. 14), β-tubulin (FIG. 21), GST-π (25C), ezrin (FIG. 22C), galectin-1 (FIG. 15C), HnRNP (FIG. 46C), MRP1 (FIG. 3) and SOD (FIG. 40). As seen above, these marker genes were identified in drug-resistant breast cancer cell lines. These results indicate that these capture probes have utility for identifying drug resistance in multiple cell types. In particular, annexin-1 was identified in ovarian cancer cell samples and breast cancer cell samples. The 2008 cell line also showed increased expression in the marker genes HSP90 (FIG. 7), HSP60 (FIG. 8), nucleophosmin (FIG. 13C), KAP-1 (FIG. 23), prohibitin (FIG. 36), 5C5-2 (FIG. 38), PDI/ER-60 precursor (FIG. 39C), FAS (FIG. 43), rad23 homolog β (FIG. 44), and α-tubulin (FIG. 45). These marker genes were specifically identified with ovarian cancer cell lines. This result establishes the importance of identifying the appropriate marker genes for the particular tissue for easy analysis of results, which a pan-genomic microarray would not allow due to the large number of genes on the microarray. The focused microarray in the present studies allowed for ready identification of marker genes that were increased in expression in drug-resistant ovarian cancer cell lines, but are not found in breast cancer cell lines.

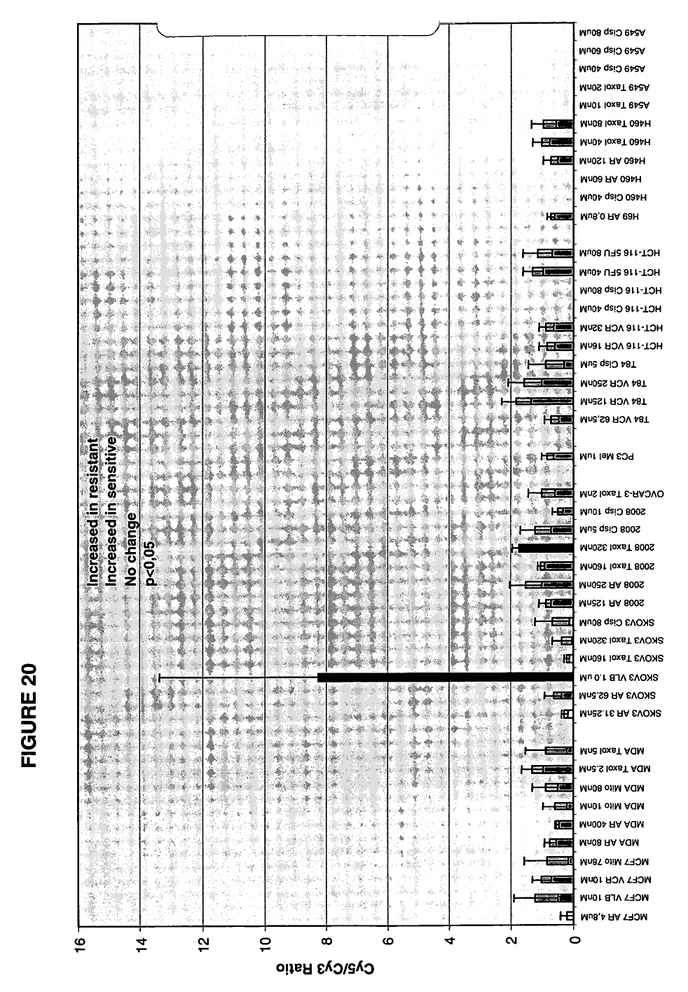

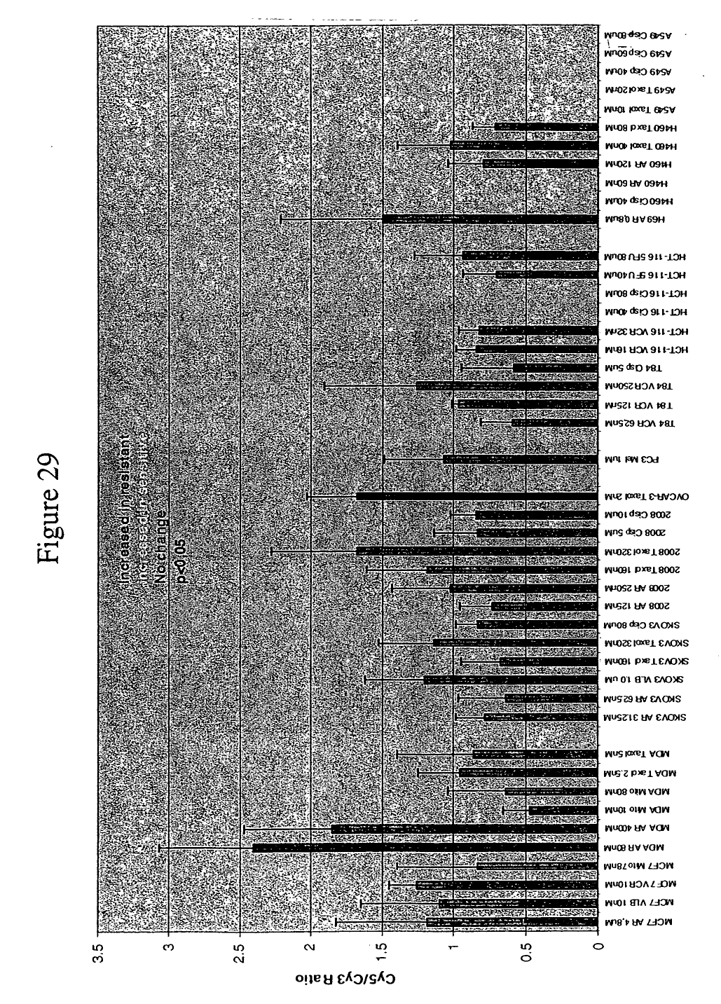

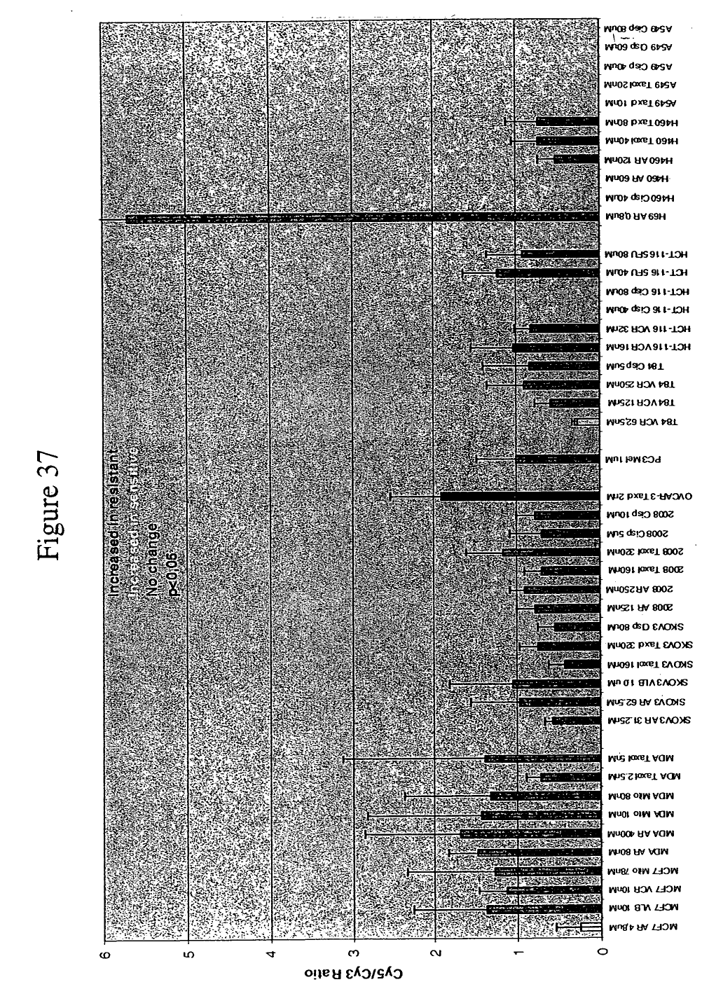

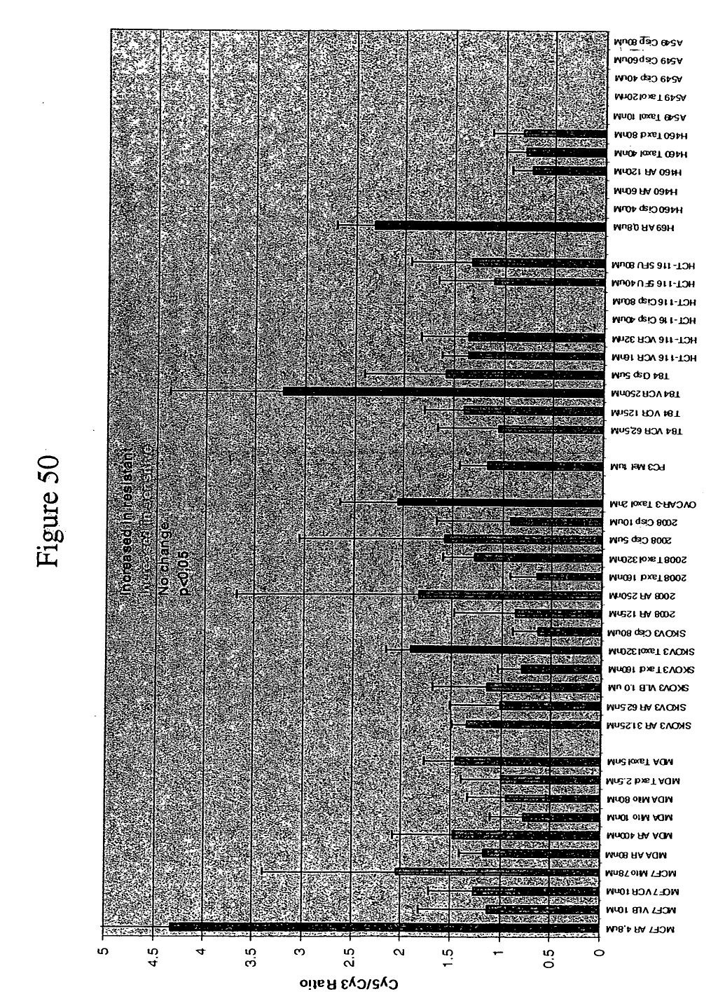

To elucidate the marker genes expressed at increased levels in 2008 and OVCAR3 ovarian cancer cell lines resistant to taxol, the focused microarray was used to compare resistant cell samples to 2008 and OVCAR3 taxol-sensitive cell lines. The OVCAR3 cell line is derived from an epithelial adenocarcinoma. The focused microarray identified ezrin (FIG. 22C), galectin-1 (FIG. 15C), HSP27 (FIG. 16), EF-2 (FIG. 29), calumenin (FIG. 50), PDI/ER-60 precursor (FIG. 39C), slc9A3R1 (FIG. 37), tropomyosin 2 (FIG. 33C), and Pgp 1 (FIG. 4). These marker genes were found to be differentially expressed between the cell lines. The 2008 taxol cell lines had increased expression of PDI/ER-60 precursor (FIG. 39C), thioredoxine peroxidase (FIG. 20), tropomyosin 2 (FIG. 33C), and Pgp 1 (FIG. 4). These markers were not identified in the OVCAR3 cell line, which indicates that the cell lines became resistant due to differing mechanisms. These results indicate that the mechanisms for drug resistance are complex, but drug resistance marker genes for individual taxol-resistant cells can be identified through the use of the focused microarray. Interestingly, cisplatinum-resistant 2008 cell lines expressed increased levels of ezrin (FIG. 22C), which was found to be increased in expression in taxol-resistant OVCAR3 cell lines. Tropomyosin 2 (FIG. 33C) showed increased expression in 2008 cisplatinum-resistant cell lines as well.

Also, the focused microarray was able to identify marker genes in vinblastin-resistant SKOV3 ovarian cancer cells. Resistant SKOV3 cells demonstrated increased expression levels of thioredoxine peroxidase (FIG. 20), PDI/ER-60 precursor (FIG. 39C), and E-FABP (FIG. 46C). The expression of thioredoxine peroxidase in resistant cells was increased in expression by greater than 5 times the level of expression found in vinblastin-sensitive cells (FIG. 20). Similarly, the level of expression for E-FABP was increased in expression greater than 6 times the levels found in vinblastin-sensitive cells (FIG. 46C). The results indicate that the increased expression of the marker genes in ovarian cell lines was significantly greater than that found in drug-sensitive cells.

To demonstrate the use of the focused microarray for lung cancer tissue, lung carcinoma cell lines were utilized in studies examining the expression of mRNA levels in lung cancer cell lines (Table 1). Adriamycin-resistant H69 lung cancer cell line samples were compared to adriamycin-sensitive samples. Labeled cDNA from the resistant and sensitive cell samples were mixed together and hybridized with the focused microarray. The marker genes identified as having increased levels of expression in resistant cell samples consisted of galectin-1 (FIG. 15C), calumenin (FIG. 50), HSP90 (FIG. 7), nucleophosmin (see FIG. 13C), p16INK4a (FIG. 48), FAS (FIG. 43), KAP-1 (FIG. 23), prohibitin (FIG. 36), 5C5-2 (FIG. 38), β-tubulin (FIG. 21), α-enolase (FIG. 30C), γ-actin (FIG. 9), annexin-1 (FIG. 12), vimentin (FIG. 10C), tropomyosin 2 β (FIG. 33C), and MRP1 (FIG. 3). These results indicate that marker genes are increased in expression in lung cancer cell lines. In particular, these marker genes were to be markers for drug resistance in other cell lines. This result illustrates that certain marker genes are increased in expression in multiple drug-resistant cell types.

To further illustrate the marker genes showing increased expression in lung cancer cells, the H460 cell line was used during studies on mRNA expression levels using the focused microarray. The adriamycin-resistant H460 cells were compared to drug-sensitive control H460 cell lines. The studies showed that galectin-1 (FIG. 15C), keratin type II (FIG. 35), and Pgp 1 (FIG. 4) had increased expression levels. It was also evident that these marker genes had showed increased expression levels in the H69 cell line.

The focused microarray was also used to determine the marker genes that were increased in expression in colon cancer cell lines. Colon cancer cell lines HCT-116 and T84, both of which are derived from colon epithelial cancers, were used for studies comparing the level of expression of marker genes in drug-resistant and drug-sensitive cell lines. The T84 vincristine-resistant cell line showed increased expression of the marker genes HSP27 (FIG. 16), Pgp 1 (FIG. 4), nucleophosmin (FIG. 13C), BIP (FIG. 11), and calumenin (FIG. 50). The increased expression levels in vincristine-resistant T84 cells ranged from 2 to 10 times the level of expression in vincristine-sensitive T84 control cells. By contrast, the HCT-116 vincristine-resistant cell line showed increased expression in the marker gene MRP1 (FIG. 3) and galectin 1 (FIG. 15C). The results establish the complexity of determining drug resistance using to small a set of capture probes. In this study, the focused microarray was able to identify marker genes in cell from the same tissue, even though the cells did not show increased expression in similar marker genes.

To determine the protein expression differences between drug-resistant and drug-sensitive cells, two-dimensional gel electrophoresis (“2D gel”) was utilized in studies of MCF7 and CEM cell lines. The studies established that changes in expression are also found at the protein level of expression in drug-resistant cells. During the studies, drug-resistant cell line samples were isolated and compared to isolated drug-sensitive controls through densitometry measurements of stained 2D gels of the cell markers.

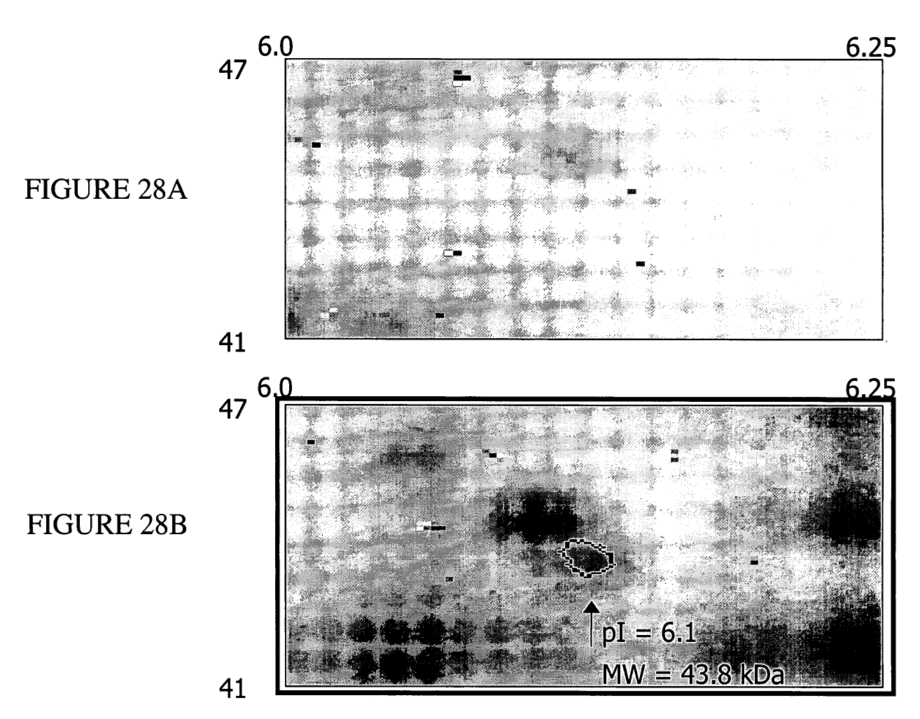

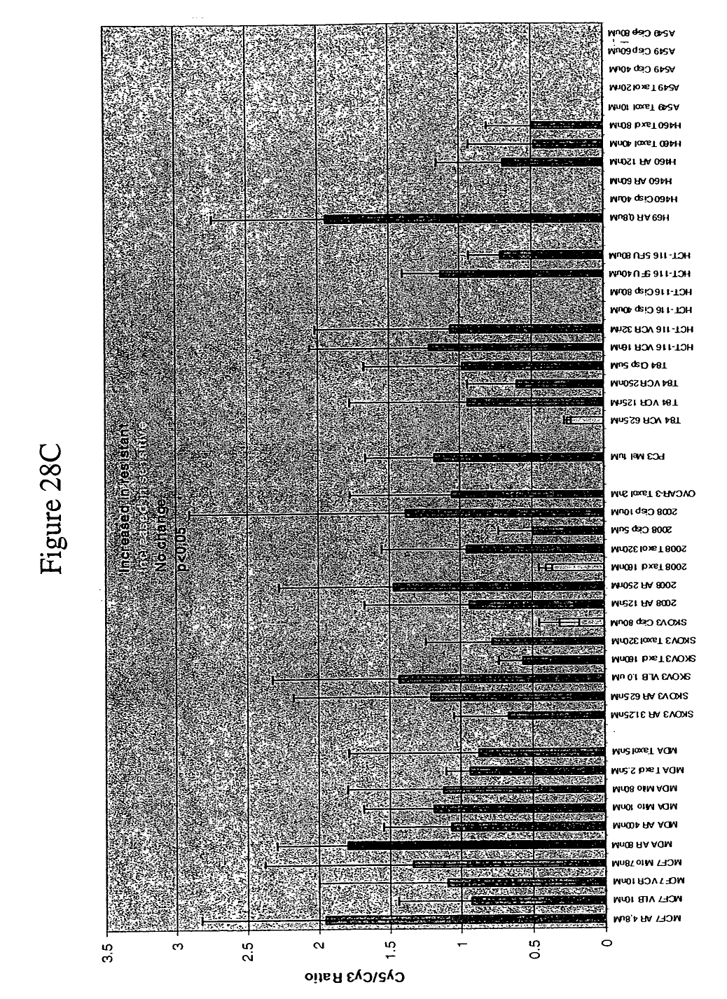

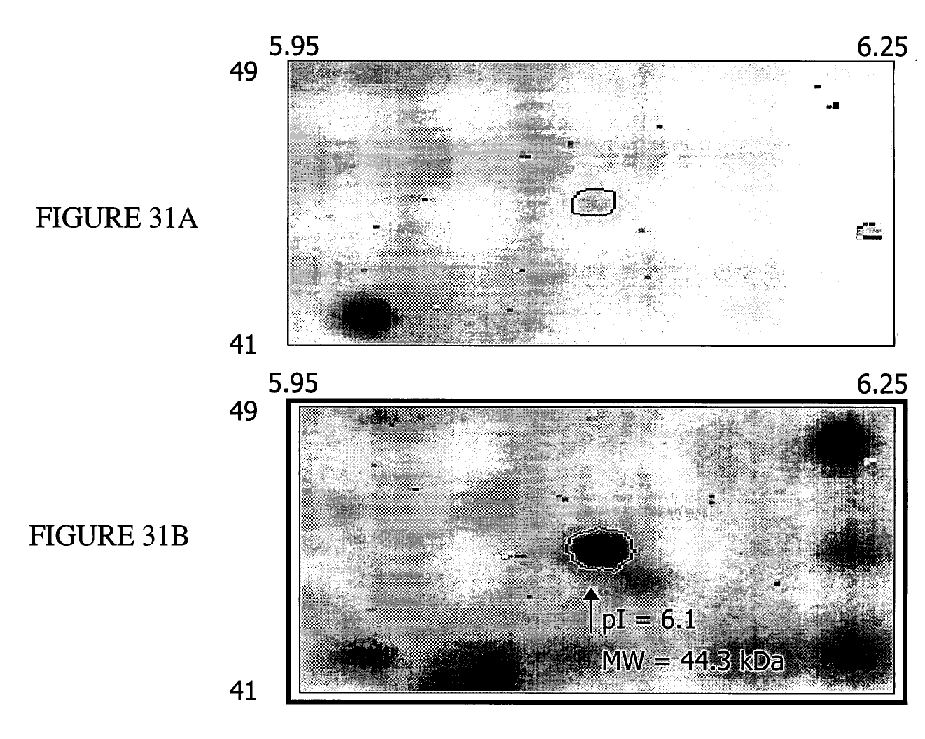

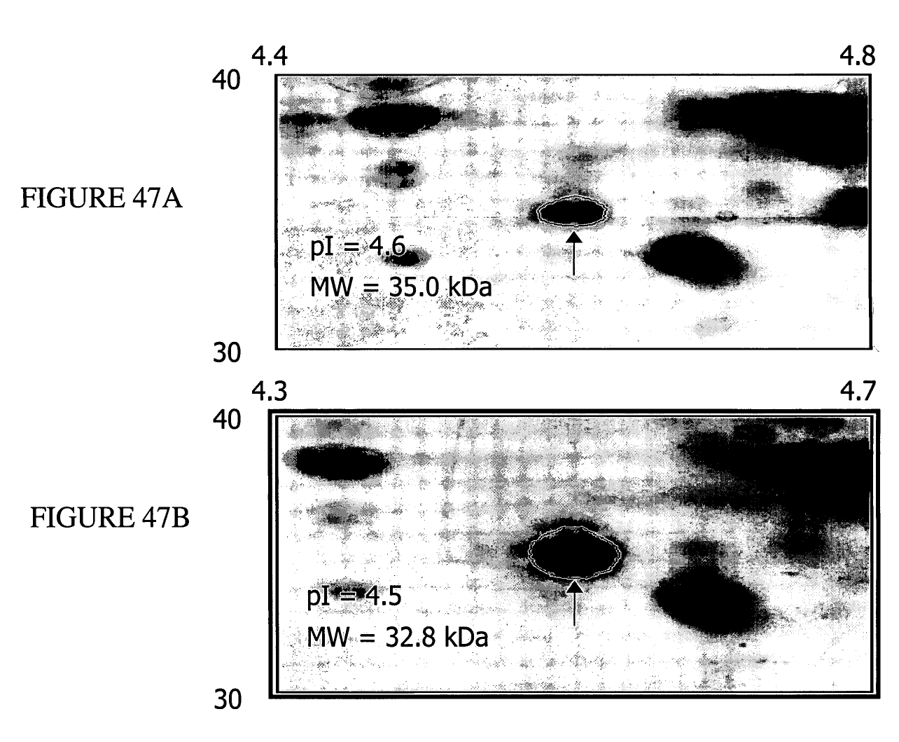

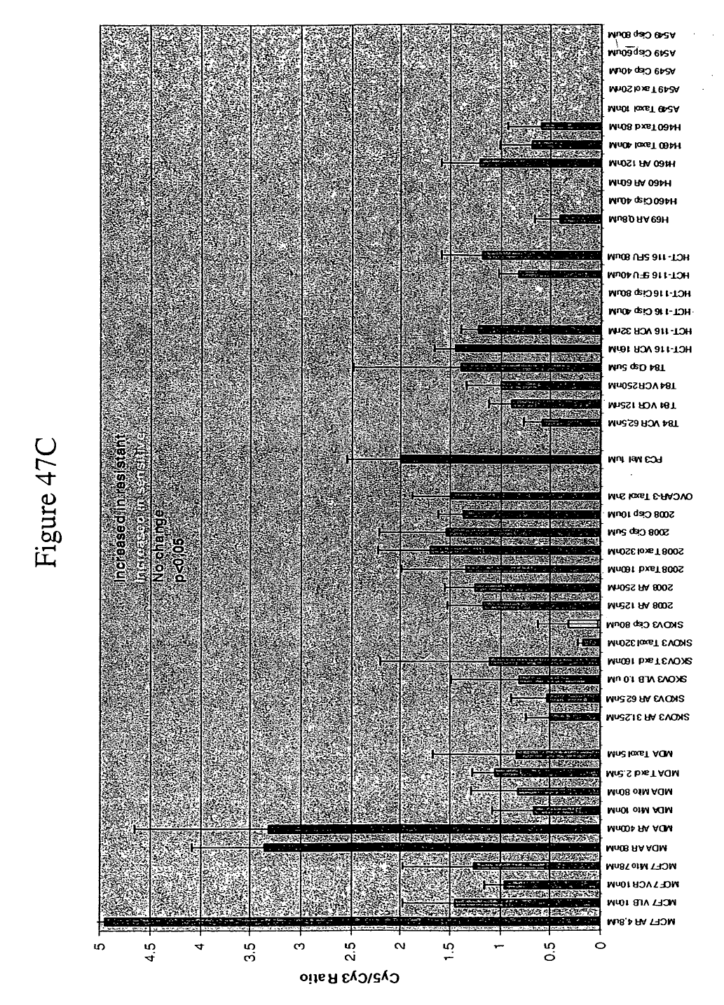

The MCF7 adriamycin-resistant cell lines showed increased cell marker expression in vimentin (FIG. 10A and 10B), galectin-1 (FIG. 15A and 15B), UCHL-1 (FIG. 17A and 17B), prosolin (FIG. 19A and 19B), and ezrin (FIG. 22A and 22B). Other markers increased in expression in adriamycin-resistant cells 2D gels included GST-π (FIG. 25A and 25B), DADEH1 (FIG. 28A and 28B), α-enolase (FIG. 30A and 30B), HnRNP (FIG. 32A and 32B), ETF3 subunit 2 (FIGS. 31A and 31B), tropomyosin 2 β (FIG. 33A and 33B), PDI-ER60 (FIG. 39A and 39B), E-FABP (FIG. 46A and 46B), and “similar to stratifin” (FIG. 47A and 47B). As the results indicate, many of these cell markers are also increased in expression at the mRNA levels. Additionally, certain cell markers such as ETF3 subunit 2 and DADEH1 have increased expression at the protein level. Such expression patterns can occur due to the various methods in which expression is modified by extrinsic signals, e.g., chemotherapeutic drug treatments (see, e.g., Giusti et al., (2004) J. Recept. Signal Transduct. Res. 24(4): 297-317). The results identify several cell markers that can be used during protein expression studies to determine whether a cancer cell sample is chemotherapeutic drug-resistant.

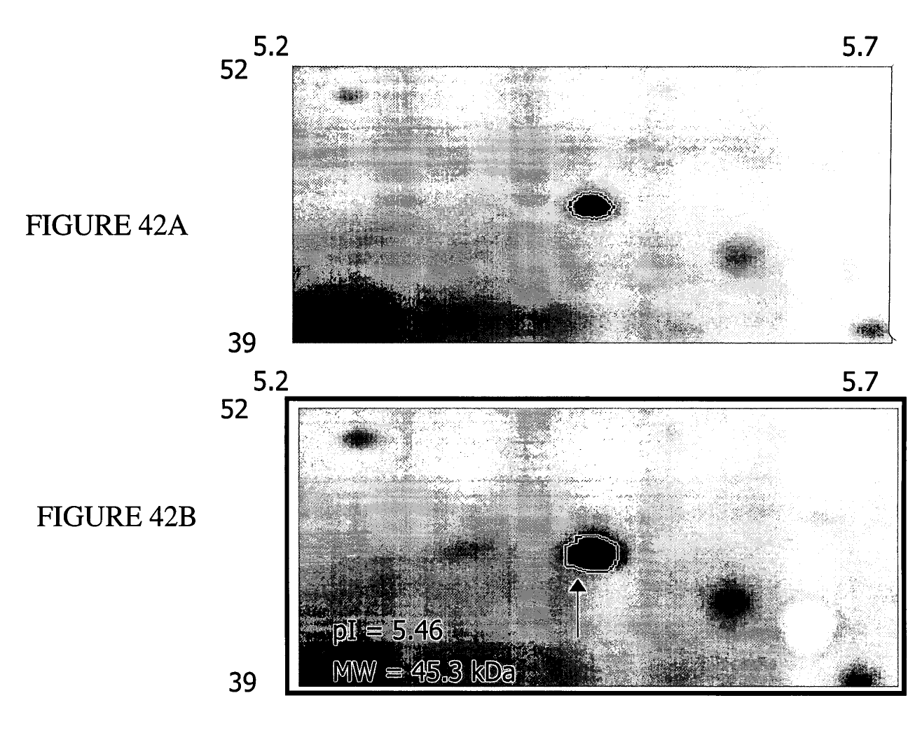

In addition to the MCF7 studies, CEM cell lines were utilized to determine cell markers that indicate chemotherapeutic drug resistance. The cell markers nucleophosmin (FIG. 13A and 13B) and NEM-sensitive factor attachment protein γ (FIG. 42) showed increased expression in vinblastin-resistant CEM cell lines compared to vinblastin-sensitive cell lines. In particular, vinblastin-resistant cells expressed NEM-sensitive factor attachment protein γ at levels determined to be 2 to 7 times greater than the levels found in vinblastin-sensitive cells. The results indicate that changes in protein expression are found in multiple cell types, which have developed resistance to chemotherapeutic drugs.

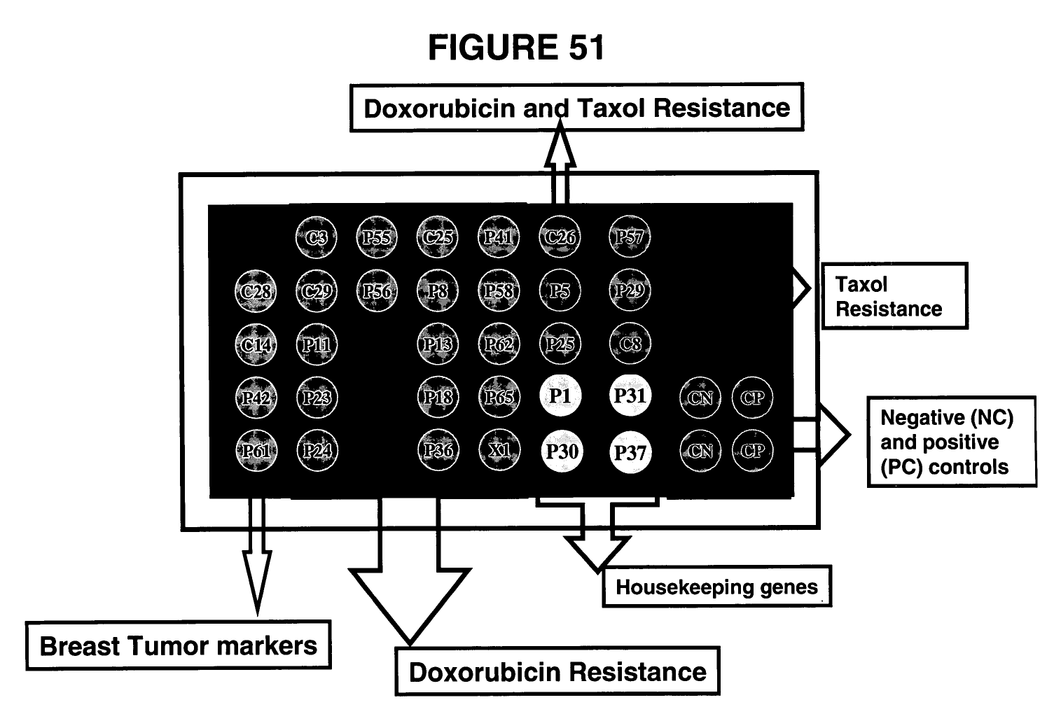

FIG. 51 shows the structure of focused microarrays for use in determining breast and ovarian chemotherapeutic drug resistance, respectively. The breast cancer resistant focused microarray is divided into several sets of capture probes, each of which can hybridize to probes generated from marker genes isolated from a cell sample or bind to cell markers isolated from a cell sample. The first set of capture probes is utilized to hybridize to marker genes that can be used to identify adriamycin resistance. Alternatively, the capture probes can be protein-binding agents capable of binding proteins from solution. The second set of capture probes is utilized to determine the expression level of marker genes that have changed expression when cancer cells are adriamycin and taxol-resistant. The third set of capture probes identifies marker genes that have altered expression levels when cells become tumorigenic. The focused microarray also contains capture probes that hybridize to probes generated from housekeeping genes that are used to normalize a signal. The housekeeping capture probes can also be protein-binding agents capable of binding housekeeping cell markers. In addition, the focused microarray has capture probes used to control for aberrant hybridization or binding of probes.

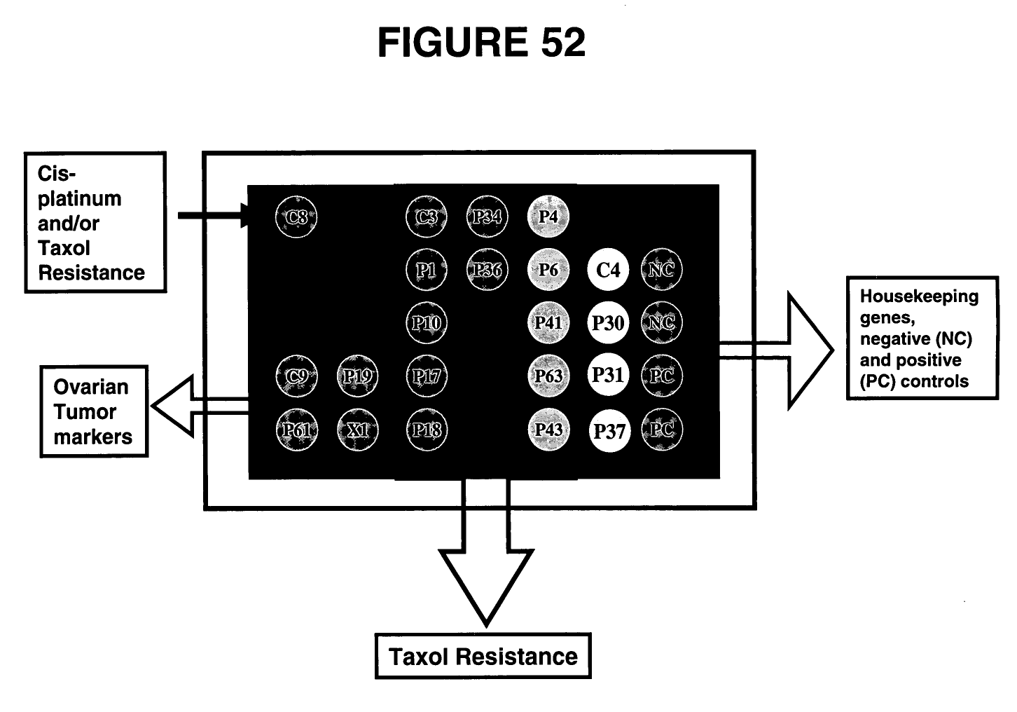

The ovarian focused microarray of FIG. 52 has a set of capture probes that are used to identify the expression level of marker genes or cell markers in a ovarian cancer cell sample. The capture probes can be used to identify taxol and cisplatinum resistance in an ovarian cancer cell sample. The focused microarray also contains a set of capture probes capable of identifying when an ovarian cell becomes tumorigenic. The set of housekeeping capture probes is used to identify the expression of housekeeping genes in the ovarian cancer cell sample, thereby allowing normalization of the microarray signal. Finally, the ovarian focused microarray has a set of positive and negative control capture probes used to control for aberrant hybridization or binding of probes.

TABLE 2
Arrangement of Markers on Breast Resistance Microarrays

| Markers on Breast Microarray | | Markers on Breast Microarray | |
|---|---|---|---|
| Position | Marker | Position | Marker |
| C28 | Keratin 19 | P57 | Cathepsin D |
| C11 | Estrogen Receptor α | P18 | EZRIN |
| C14 | c-erb-B-2/HER-2/neu | P13 | Prosolin |
| P42 | SLC9A3R1 | P53 | L-plastin |
| P61 | A-CRABP II | P60 | Calumenin |
| C17 | PCNA | P41 | Prohibitin |
| C6 | Topoisomerase IIα | P14 | Thioredoxine peroxidase 1 |
| P15 | β-Tubulin | P6 | B23 |
| P26 | CBX3 | P29.1 | PDI |
| C8 | Cathepsin B | P54 | α-tubulin |
| C27 | BAX | P65 | Interleukine 18 precursor |
| P59 | APRT | C2 | MRP1 |
| P2 | γ-actin | P12 | ATP synthase β |
| P58 | p16INK4a | P21 | UCK |
| X1 | Prefoldin subunit 1 | P62 | MYL16 |
| P8 | HSC 70 | C4 | FABP7 |
| P48 | n-ethyl-sensitive factor γ | P30 | DADEH1 |
| P31 | EF2 | P1 | HSP 60 |
| P37 | EIF-4B | | |

TABLE 3
Arrangement of Markers on Ovarian Resistance Microarrays

| Markers on Ovarian Microarray | | Markers on Ovarian Microarray | |
|---|---|---|---|
| Position | Marker | Position | Marker |
| C24 | Prostasin | P26 | CBX 3 |
| P61 | A-CRABP II | C9 | p53 |
| C29 | Cytokeratin 7 | C14 | c-erb-B-2/HER-2/neu |
| P8 | HSC 70 | P44 | PDI/ER-60 precursor |
| P19 | KAP-1 | C4 | FABP7 |
| P62 | MYL 16 | C7 | FABP3 |
| X1 | Prefoldin subunit 1 | P30 | DADEH1 |
| P59 | APRT | P31 | EF2 |
| P37 | EIF-4B | | |

The results using the microarray demonstrate that marker genes and cell markers have increased expression in chemotherapeutic drug-resistant cancer cells compared to controls, and that the expression of cell markers and marker genes can be identified with capture probes affixed to a solid surface.

The cell markers of the invention have been identified using 2D gel technology. At the protein level, cell markers showed increased levels of expression in chemotherapeutic drug-resistant cancer cells relative to chemotherapeutic drug-sensitive controls. The cell markers are from a group such as ezrin, HnRNP, UCHL-1, E-FABP, “similar to stratifin”, vimentin, galectin-1, GST-π, α-enolase, NEM factor attachment protein γ, PDI/ER-60 precursor, Rad23 homolog β, prosolin, tropomyosin 2β, nucleophosmin and ETF3 subunit 2. The antibody microarray identifies cell markers that show higher levels of expression in drug-resistant cancer cells relative to drug-sensitive controls. When determining drug-resistance in a cancer cell sample, the results from the antibody microarray should be the same as those obtained from 2D gel studies of protein expression.

The microarrays according to the invention can be used to perform clinical studies on tumor tissues isolated from patients are performed using the focused microarray. For example, breast tumors isolated from patients show results similar to those found in the breast cancer cell line studies. Chemotherapeutic drug-resistant breast cancer cells from patient samples show increased expression in a plurality of markers identified by a capture probes on the focused microarray. Examples of potential nucleic acid marker genes that can be identified in a study of resistant breast cancer clinical samples are γ-actin, vimentin, HSC70, galectin-1, prosolin, β-tubulin, GST-Π, α-enolase, HSP27, and SOD. Furthermore, a plurality of marker genes such as UCHL-1, ezrin and “similar to stratifin” can potentially show increased expression in the drug-resistant cells due to their increased expression in breast cancer cell lines resistant to chemotherapeutic drug treatment.

Further studies can be performed on clinical samples from ovarian tissue. The samples are, in some cases, drug-resistant to one or more chemotherapeutic drugs. The focused microarray identifies marker genes that are increased in expression in the ovarian tumor tissues when compared to a drug-sensitive control ovarian cancer sample. These markers are from a group such as Pgp 1, P53, annexin-1, ezrin, KAP-1, HnRNP, E-FABP, HSP27, SOD, γ-actin, vimentin, HSC70, galectin-1, prosolin, β-tubulin, α-enolase, HSP90, HSP60, nucleophosmin, FAS, Rad23 homolog β, α-tubulin, MRP1, keratin type II, tropomyosin 2β, prohibitin, calumenin, 5C5-2, SLC9A3R1, pyrophosphatase inorganic, MB-COMT, EF2, PDI, and PDI/ER 60 precursor protein. These marker genes exhibit increased expression in drug-resistant ovarian cancer cell lines. It is likely that these genes can exhibit the same characteristics in tumor tissues isolated from patients.

### EXAMPLES

This invention is further illustrated by the following examples, which should not be construed as limiting. Those skilled in the art will recognize, or be able to ascertain, using no more than routine experimentation, numerous equivalents to the specific substances and procedures described herein. Such equivalents are intended to be encompassed in the scope of the claims that follow the examples below.

### Example 1

### Preparation and use of the Focused Microarray on Drug Resistant Cell Lines

1. Total RNA Isolation and cDNA Labeling

Drug-resistant mRNA samples were isolated from MCF7 cell lines (ATCC, #HTB-22) that were resistant to adriamycin concentrations of 4.8 μM (Table 1). Resistant cell lines and their sensitive counterparts were grown in cell specific medium conditions at 37° C./5% CO₂. Drug-sensitive cell samples were isolated from MCF7 (ATCC, #HTB-22), and were used as control cell samples (Table 1). Cell lysis and RNA extraction was done with the RNEasy kit, (# 74104) (Qiagen, Inc., Valencia, Calif.) following the manufacturer's protocol. RNA was quantified by spectrophotometry using an Ultrospec 2000 spectrophotometer (Amersham-Biosciences, Corp., Piscataway, N.J.). RNA samples were dissolved in 10 mM Tris, pH 7.5 to determine the A_{260/280} ratios. Samples with ratios between 1.9 and 2.3 were kept for probe preparation, while samples with ratios lower than 1.9 were discarded. RNA samples were dissolved in 1 μl DEPC-H₂O for total nucleic acid quantification. Total RNA from control and treated samples was dried by speed vacuum using a Heto Vacuum centrifuge system (KNF Neuberger, Inc., Trenton, N.J.) at varying time intervals. The total RNA was resuspended in 10 μl of DEPC-H₂O and stored at −20° C. until the labeling reaction.

First strand cDNA labeling was accomplished using 1-15 μg total RNA (depending on the cell lines to be tested) for the resistant and the sensitive cell lines separately. Total RNA was incubated with 4 ng control positive Arabidopsis thaliana RNA, 3 μg of Oligo (dT)₁₂₋₁₈ primer (# Y01212) (Invitrogen, Corp., Carlsbad, Calif.), 1 μg PdN6 random primer (Amersham, #272166-01) for 10 min. at 65° C., and immediately put on ice for 1 min. The mixture was then diluted in 5× First strand buffer (250 mM Tris-HCl, pH 8.3; 375 mM KCl; 15 mM MgCl₂) containing 0.1 M DTT, 0.5 μM dNTPs mix (dTTP, dGTP, dATP) (Invitrogen, #10297-018), 0.05 μM dCTP (Invitrogen, #10297-018), 5 μM Cy3-dCTP (#NEL 576) (NEN Life Science/Perkin Elmer, Boston, Mass.), 2.5 μM Cy5-dCTP (#NEL 577) (NEN Life Science/Perkin Elmer, Boston, Mass.) and 400 units SuperScript III RNAse H⁻ RT (Invitrogen, #I 8064-014). After incubating the reaction mixture for 5 min. at 25° C., the reaction mixture was incubated at 42° C. for 90 min. Finally, a total of 400 units of SuperScript II RNAse H⁻ RT (Invitrogen, #18064-014) were added and the reaction was incubated at 42° C. for another 90 min.

Digestion of the labeled cDNA with 5 units RNAse H (#M0297S) (NEB, Beverly, Mass.) and 40 units RNAse A (Amersham, # 70194Y) was done at 37° C. for 30 min. The labeling probe was purified with the QIAquick PCR purification kit (Qiagen, Inc.) protocol with some modifications. Briefly, the reaction volume was completed to 50 μl with DEPC-H₂O and 2.7 μl of 12 M NaOAc pH 5.2 was added. The reaction was diluted with 200 μl PB buffer, put on the purification column, spun 15 sec. at 10 000 g, followed by 3 washes of 500 μl PE buffer (15 sec.; 10 000 g) and eluted 2 times in 50 μl DEPC-H₂O total (1 min.; 10 000 g). Frequency of incorporation and amount of cDNA labeled produced were evaluated for both labeled dCTPs by spectrophotometer (Ultrospec 2000, Pharmacia Biotech) at A_{260 nm}, A_{550 nm} and A_{650 nm}. The labeling material was dry by speed vacuum (Heto Vacuum centrifuge system, LaboPort) and resuspended in 3.75 μl H₂O total for both Cy5 (resistant cell line) and Cy3 reactions (sensitive cell line).

2. Capture Probe Preparation

Capture probes, approximately 68 nucleotides in length, corresponding to targets of interest were designed using sequences showing less identity base to base (<30%) with other coding sequences (cds) submitted to NCBI bank. The comparisons between sequences were done by BLAST research (www.ncbi.nlm.nih.gov/BLAST). For BioChip ver1.0 and ver2.0, a basic melting point temperature at a salt concentration of 50 mM Na⁺ (Tm) for each capture probe was calculated: the overall average was 76.97° C.±3.72° C. GC nucleotide content averaged 51.2%±9.4%. For the present invention, two negative controls (68 bp of the antisense cds of the BRCP and nucleophosmin targets) were synthesized.

The targets present on the oligonucleotide array were: Breast cancer resistance protein (BCRP) (gi # 12414050) 1-68 bp of cds; Multidrug resistance-associated protein 1 (MRP-1) (gi# 9955961) 303-370 bp of cds; Multidrug resistance-associated protein 1 (MRP-1)(gi# 9955961) 4501-4568 bp of cds; P-glycoprotein 1 (Pgp 1) (gi# 7669470) 201-268 bp of cds; Pgp 1l (gi# 7669470) 3061-3128 bp of cds; Fatty acid binding protein 7 (FABP7) (gi# 16950660) 330-398 bp of cds; Lung resistant protein (gi#19577289) 2400-2468 bp of cds; topoisomerase IIα (gi# 19913405) 4500-4568 bp of cds; Fatty acid binding protein 3 (FABP3) (gi# 10938020) 334-402 bp of cds; cathepsin β (gi# 22538429) 942-1010 bp of cds; p53 (gi# 35213) 1073-1141 bp of cds; Heat shock protein 90 (HSP90) (gi# 184422) 2100-2168 bp of cds; Heat shock protein 60 (HSP60) (gi# 14730099) 1801-1868 bp of cds; γ-actin (gi# 11038618) 1000-1068 bp of cds; Vimentin (gi# 4507894) 1-68 bp of cds; vimentin (gi# 4507894) 1261-1328 bp of cds; BIP (gi# 6470149) 1631-1698 bp of cds; annexin-1/p-40 (gi# 4502100) 1-68 bp of cds; p-40 (gi# 4502100) 823-890 bp of cds; nucleophosmin (gi# 10835062) 543-610 bp of cds; nucleophosmin (gi# 10835062) 813-880 bp of cds; Heat shock 70 kDa protein 8 (HSC70)(gi# 5729876) 1451-1518 bp of cds; Heat shock 70 kDa protein 8 (HSC70) (gi# 5729876) 1645-1712 bp of cds; galectin-1 (gi# 6006015) 341-408 bp of cds; Heat shock protein 27 (HSP27) (gi# 4996892) 61-128 bp of cds; ubiquitin C-term hydrolase isozyme L1 (UCHL-1) (gi# 18558293) 213-280 bp of cds; ubiquitin C-term hydrolase isozyme L1 (UCHL-1) (gi# 18558293) 471-538 bp of cds; ATP synthase β (gi# 179280) 1033-1200 bp of cds; prosolin (gi# 13518023) 351-418 bp of cds; thioredoxine peroxidase 1 (gi# 440307) 529-597 bp of cds; β-tubulin (gi# 3387928) 400-468 bp of cds; guanine nucleotide binding protein, β polypeptide 3 (GNBP β3) (gi# 183412) 350-398 bp of cds; MB-COMT (gi# 6466451) 101-168 bp of cds; EZRIN (gi# 21614498) 1011-1078 bp of cds; KAP-1 (gi# 1699026) 1-68 bp of cds; UMP-CMP kinase (gi# 5730475) 391-458 bp of cds; alternative splicing factor (ASF-2) (gi# 179073) 811-878 bp of cds; pyrophosphatase inorganic (gi# 12735403) 533-600 bp of cds; GST-π α chain (gi# 31947) 565-633 bp of cds; ATP synthase D (gi# 5453558) 213-280 bp of cds; chromobox homolog 3 (CBX3) (gi# 15082257) 31-98 bp of cds; protein disulfide isomerase precursor (PDI) (gi# 20070124) 543-610 bp of cds; dimethylarginine dimethylaminohydrolase 1 (DADEH1) (gi# 6912327) 399-456 bp of cds; dimethylarginine dimethylaminohydrolase 1 (DADEH1) (gi# 6912327) 651-718 bp of cds; Elongation factor 2 (EF2) (gi# 181968) 833-900 bp of cds; α-enolase (gi# 2661038) 943-1010 bp of cds; eukaryotic translation factor 3 subunit 2 (ETF3-subunit 2) (gi# 4503512) 833-900 bp of cds; heterogenous nuclear ribonucleoprotein F (HnRNP) (gi# 14141150) 771-838 bp of cds; tropomyosin 2 β (gi# 20070122) 550-617 bp of cds; eukaryotic translation initiator factor 4B (EIF 4B) (gi# 4503532) 901-968 bp of cds; hepatoma derived growth factor (gi# 4758515) 393-460 bp of cds; keratin type II cytoskeletal (gi# 12737278) 1171-1238 bp of cds; prohibitin (gi# 6031190) 713-780 bp of cds; solute carrier family 9 isoform 3 regulatory factor 1 (slc9A3R1) (gi# 4759139) 631-738 bp of cds; 5C5-2 (gi# 4324471) 141-208 bp of cds; protein disulfide isomerase ER-60 precursor (PDI-ER60) (gi# 1208427) 833-900 bp of cds; β-spectrin (gi# 338439) 3100-3168 bp of cds; β-spectrin (gi# 338439) 4000-4068 bp of cds; Superoxide dismutase (SOD) (gi# 4507148) 391-458 bp of cds; caspase recruitment domain protein 14 (gi# 13653996) 895-968 bp of cds; N-ethylmaleimide-sensitive factor attachment protein γ (NEM-sensitive factor attachment protein γ) (gi# 4505330) 732-800 bp of cds; fatty acid synthase (FAS) (gi# 4758341) 1-68 bp of cds; fatty acid synthase (FAS) (gi# 4758341) 7233-7300 bp of cds; triosephosphate isomerase (TPI) (gi# 339840) 400-467 bp of cds; Rad23 homolog β (gi# 19924138) 900-968 bp of cds; L-Plastin (gi# 16307447) 1600-1668 bp of cds; α-tubulin (gi# 3420928) 1288-1356 bp of cds; fatty acid binding protein, epidermal (E-FABP) (gi# 4557580) 1-68 bp of cds; fatty acid binding protein, epidermal (E-FABP) (gi# 4557580) 341-408 bp of cds; “similar to stratifin” (gi# 16306736) 314-382 bp of cds; cathepsin δ (gi# 18577791) 411478 bp of cds; p16INK4a (gi# 16753086) 168 bp of cds; p16INK4a (gi# 16753086) 1-68 bp of cds; adenine phosphoribosyltransferase (APRT) (gi# 4502170) 100-168 bp of cds; calumenin (gi# 14718452) 880-943 bp of cds.

The capture probes were synthesized by the BRI Institute (Biotechnology Research Institute, Clear Water Bay, Kowloon, Hong Kong, China) with the Expedilite™ Synthesizer at a coupling efficiency of over 99.5% (Applied Biosystems, Foster City, Calif.). The oligonucleotides were verified by polyacrylamide gel electrophoresis. Oligonucleotide quantification was done by spectrophotometry at A_{260 nm}.

3. Printing of Capture Probes and Production of the Focused Microarray

Prior to printing of capture probes, different dilutions of Arabidopsis thaliana chlorophyll synthetase G4 DNA (undiluted solutions at 0.15 μg/μl and at 0.2 μg/μl; 1:2; 1:4; 1:8; 1:16) were printed on each grid as a positive control, and for normalization of results. Preparation of Arabidopsis thaliana control capture probes was performed as follows. Briefly, five micrograms of a Midi preparation using a HiSpeed™ Plasmid Midi kit (Qiagen, Inc.) of the Arabidopsis thaliana plasmid (gift of BRI) was digested with 40 units of Sac I enzyme (NEB) for 2 hr. at 37° C., purified with the QIAquick PCR purification kit (Qiagen,) and verified by 1% agarose migration. In vitro transcription of 2 μg Sac I digestion was performed in 10× transcription buffer (400 mM Tris-HCI, pH 8.0; 60 mM MgC12; 100 mM DTT; 20 mM Spermidin) containing 2 μl of 10 mM NTP mix (Invitrogen), 20 units RNAse OUT (Invitrogen, #10777-019) and 50 units T7 RNA polymerase (NEB) for approximately 2 hr. to 30 hr. at 37° C. The reaction was then treated with 2 units DNAse I (Invitrogen) in 10× DNAse buffer (200 mM Tris-HCI pH 8.4; 20 mM MgC1₂; 500 mM KC1) for 15 min. at 37° C. The RNA was cleaned with the RNEasy kit (Qiagen) and quantified by spectrophotometry using an Ultrospec 2000 (Amersham Biosciences, Corp.).

After the control capture probes were generated and printed, the capture probes complementary to marker genes from the cancer cell samples were printed at concentrations of 25 μM in 50% DMSO on CMT-GAPS II Slides (# 40003) (Coming, 45 Nagog Park, Acton, Mass.) by the VersArray CHIP Writer Prosystems (BioRad Laboratories) with the Stealth Micro Spotting Pins (#SMP3) (Telechem International, Inc., Sunnyvale, Calif.). Each capture probe was printed in triplicate on duplicate grids. Buffer and Salmon Testis DNA (Sigma D-7656) were also printed for the BioChip analysis step. After printing was completed, the slides were dried overnight by incubation in the CHIP Writer chamber. Chips were then treated by UV (Stratagene, UV Stratalinker) at 600 mjoules and baked in an oven for 6-8 hr.

4. Ouality Control of Focused Microarray

Prior to testing the invention on cancer cell samples, the focused microarray was tested at the BRI Institute (Kowloon Bay, Hong Kong). One slide for each printed batch was quality control tested using a terminal deoxynucleotidyl transferase (Tdt)-mediated nick end labeling assay protocol (see, e.g., Yeo et. al., (2004) Clin. Cancer Res. 10(24): 8687-96). Additionally, controls were performed to verify the specificity of the hybridization using three independent grids on the same focused microarray.

As a first quality control, a test was done by the BRI Institute on one slide for each batch printed with the following Tdt transferase protocol. Briefly, the slide was prehybridized in a Hybridization Chamber (#2551) (Coming, Inc., Life Sciences, 45 Nagog Park, Acton, Mass.) with 80 μl of preheated prehybridization buffer (5×SSC (750 mM NaCl; 75 mM sodium citrate); 0.1% SDS; 1% BSA (Sigma, #A-7888) at 37° C. for 30 min. Slides were washed in 0.1×SSC (15 mM NaCl; 1.5 mM sodium citrate) and air-dried. Fifty micro-liters of TdT reaction mixture [5× TdT buffer (125 mM Tris-HCl, pH 6.6, 1 M sodium cacodylate, 1.25 mg/ml BSA); 5 mM CoCl₂; 1 mM Cy3-dCTP (NEN Life Science, NEL 576); 50 units TdT enzyme (#27-0730-01) (Amersham BioSciences)], was added to the entire area of the BioChip. The slide was incubated in the Hybridization Chamber for 60 min. at 37° C. following by a first wash in 1×SSC (150 mM NaCl; 15 mM sodium citrate)/0.2% SDS (preheated at 37° C.) for 10 min., a second wash of 5 min. in 0.1×SCC (15 mM NaCl; 1.5 mM sodium citrate)/0.2% SDS at room temperature and finally a last wash of 5 min. at room temperature in 0.1×SSC (15 mM NaCl; 1.5 mM sodium citrate). The slide was scanned with the ScanArray™ Lite MicroArray Scanner (Packard BioSciences, Perkin Elmer, San Jose, Calif.).

As a second quality control step, the PARAGON™ DNA Microarray Quality Control Stain kit (Molecular Probes) was incubated with the microarray according to the manufacturer's recommendations (FIGS. 1A-1C)

5. Focused Microarray Hybridization with Labeled cDNA Probes

Focused microarray slides were pre-washed before the prehybridization step as follows. First, slides were washed for 20 min. at 42° C. in 2×SSC (300 mM NaCl; 30 mM sodium citrate)/0.2% SDS under agitation. The second wash was for 5 min. at room temperature in 0.2×SSC (30 mM NaCl, 3 mM Sodium citrate) under agitation, and then followed by a wash for 5 min. at room temperature in DEPC-H₂O with agitation. The slides were spin dried at 1000 g for 5 min. and prehybridized in Dig Easy Hyb Buffer (#1,603,558) (Roche Diagnostics Corporation, Indianapolis, Ind.) containing 400 μg Bovine Serum Albumin (Roche, #711,454) at 42° C. in humid chamber for 3 hr. then washed 2 times in DEPC-H₂O, and once in Isopropanol (Sigma, 1-9516) and spun dry at 1000 g for 5 min.

To the mixed Cy5/Cy3 probe, 15 μg Baker tRNA (#109,495) (Roche Diagnostics Corp., Indianapolis, Ind.) and 1 μg Cot-1 DNA (Roche, #1,581,074) were added and the probe was incubated 5 min. at 95° C., put on ice for 1 min., and diluted with 14 μl Dig Easy Hyb buffer (Roche, #1,603,558). After a 2 min. spin at 100 g, the probe was incubated at 42° C. for at least 5 min.

The three supergrids on the slide were separated by a Jet-Set Quick Dry TOP Coat 101 line (#FX268) (L'Oreal, Paris, FR) (FIGS. 1A-1C). Each probe was added to its respective supergrid and covered by a preheated (42° C.) coverslip (Mandel, #S-104 84906). The slide was incubated at 42° C. in humid chamber for at least 15 hr.

The coverslips were removed by dipping in 1×SSC (150 mM NaCl; 15 mM sodium citrate)/0.2% SDS solution preheated at 50° C.). The slide was washed three times for 5 min. with agitation in 1×SSC (150 mM NaCl; 15 mM sodium citrate)/0.2% SDS solution preheated at 50° C.), and then washed three times with agitation in 0.1×SSC (15 mM NaCl; 1.5 mM sodium citrate)/0.2% SDS solution preheated at 37° C.). Finally, the slide was washed once in 0.1×SSC (15 mM NaCl; 1.5 mM sodium citrate) with agitation for 5 min. The slide was dipped several times in DEPC-H₂O and spun dry at 1000 g for 5 min.

6. Scanning and Statistical Analysis

The slides were scanned with a ScanArray™ Lite MicroArray Scanner (Packard BioSciences, Perkin Elmer, San Jose, Calif.) and the analysis was performed with a QuantArrayR Microarray Analysis software version 3.0 (Packard BioSciences, Perkin Elmer, San Jose, Calif.).

The QuantArray® data results were analyzed according to the following procedures. All analysis of the results was performed with the spot background subtracted values for Cy5 and Cy3. Spots with lower signal ratio to noise lower than 1.5 were discarded. Normalization of the ratios with the spike positive control (Arabidopsis thaliana) was done to have a ratio equal to one for that control on each slide. Slides were discarded on which the negative and/or positive controls did not work. Also, slides were discarded with high background and with different mean no offset correction (ArrayStat software). Mean for each target was calculated with at least six different experiments (including two reciprocal labeling reactions), each experiment using different total RNA preparations. Statistical analysis was accomplished with the ArrayStat 1.0 (Imaging Research Inc., Brock University, St. Catherine's, Ontario, Calif.). A log transformation of the ratio data is followed by a Student T test for two independent conditions using a proportional model without offsets at a p<0.05 threshold. Significant increases (ratio Cy5/Cy3 higher than 1.5) or decreases (ratio Cy5/Cy3 lower than 0.5) were considered to be significant if the p value was lower than 0.05.

### Example 2

### Identification of Drug Resistant Cancer in Patient Samples

1. Patient Samples and RNA Isolation

Patients with ductal adenocarcinoma were included in the breast cancer data group. Asterand pathologists confirmed pathological diagnosis. Standard clinical and pathological reports were available for each patient included in this study. Breast normal total RNA was purchased from Stratagene (La Jolla, Calif.). The first total RNA sample was from a 56 year-old woman. Breast total RNA pool was purchased from (Biochain Inc, Hayward, Calif.).

All patient material was purchased from (Asterand, Inc., Detroit, Mich.). Asterand Inc extracted total RNA from frozen tissue samples with a derived Trizol extraction procedure. Total RNA was then treated with RNA-free DNAse I and purified with the RNEasy kit (Qiagen GmbH, Hilden, Germany). The isolated RNA was analyzed with an Agilent BioAnalyzer (Agilent Technologies, Palo Alto, Calif.). Total RNA was quantified with a spectrophotometer and A_{260/280 nm} ratio was calculated using an Ultrospec 2000 (Amersham-Pharmacia Corp., Piscataway, N.J.).

2. Use of the Focused Microarray to Identify Drug-Resistant Cancer Cell Samples

The capability of the focused microarray to identify chemotherapeutic resistance in a cancer cell sample isolated from a patient will be determined by practicing the following example. Focused microarray slides will be pre-washed before the prehybridization step as follows. First, slides will be washed for 20 min. at 42° C. in 2×SSC (300 mM NaCl; 30 mM sodium citrate)/0.2% SDS under agitation. The second wash is for 5 min. at room temperature in 0.2×SSC (30 mM NaCl, 3 mM sodium citrate) under agitation, and then the slide will be washed for 5 min. at room temperature in DEPC-H₂O with agitation. The slides spin at 1000 g for 5 min. until dry and prehybridize in Dig Easy Hyb Buffer (#1,603,558) (Roche Diagnostics Corp., Indianapolis, Ind.) containing 400 μg bovine serum albumin (Roche, #711,454) at 42° C. in humid chamber for 3 hr. The slide is washed twice in DEPC-H₂O, then once in isopropanol (#1-9516) (Sigma-Aldrich Co., St. Louis, Mo.) and is spun dry at 1000 g for 5 min.

To the mixed Cy5/Cy3 probe, add 15 μg Baker tRNA (Roche, #109,495) and 1 μg Cot-1 DNA (Roche, #1,581,074) and incubate the probe for 5 min. at 95° C. After the incubation is complete, the mixture is put on ice for 1 min. and diluted with 14 μl Dig Easy Hyb buffer (Roche, #1,603,558). After a 2-min. spin at 100 g, the probe is incubated at 42° C. for at least 5 min. The probe mixture is added to the slide and a coverslip is placed over the mixture. The slide should be incubated at 42° C. in humid chamber for at least 15 hr.

The coverslips are removed by dipping in 1×SSC (150 mM NaCl; 15 mM sodium citrate)/0.2% SDS solution preheated at 50° C. The slides are washed three times for 5 min. with agitation in 1×SSC (150 mM NaCl; 15 mM sodium citrate/0.2% SDS solution preheated at 50° C.). The slides are also washed three times with agitation in 0.1×SSC (15 mM NaCl; 1.5 mM sodium citrate)/0.2% SDS solution preheated at 37° C. and once with agitation in 0.1×SSC (15 mM NaCl; 1.5 mM sodium citrate) for 5 min. Slides are dipped several times in DEPC-H2O. Slides are dried by centrifugation at 1000 g for 5 min.

3. Scanning and Statistical Analysis

The slide scanning is performed with a ScanArray™ Lite MicroArray Scanner (Packard BioSciences, Perkin Elmer, San Jose, Calif.). The analysis is performed with a QuantArrayR Microarray Analysis software version 3.0 (Packard BioSciences, Perkin Elmer, San Jose, Calif.).

The results are analyzed using QuantArray® software according to the following procedures. All analysis of the results subtracts the spot background values for Cy5 and Cy3 from the experimental results. Spots with lower signal ratio to noise lower than 1.5 should be discarded. Normalization of the ratios with the spike positive control (Arabidopsis thaliana) allows a ratio equal to one for that control on each slide. Slides are discarded on which the negative and/or positive controls do not work. Also, slides are discarded with high background and with different mean no offset correction as determined by ArrayStat software. The calculation of means for each target requires at least six different experiments (including two reciprocal labeling reactions), each experiment uses different total RNA preparations. Statistical analyses are accomplished with the ArrayStat 1.0 (Imaging Research Inc.). A log transformation of the ratio data is followed by a Student T test for two independent conditions using a proportional model without offsets at a p<0.05 threshold. Significant increases (ratio Cy5/Cy3 higher than 1.5) or decreases (ratio Cy5/Cy3 lower than 0.5) are significant if the p value was lower than 0.05.

### Example 3

### Two Dimensional Gel Electrophoretic Analysis of Cell Marker Expression in Drug Resistant Cell Lines

1. Preparation of Cell Extracts

Briefly, cultured cells were rinsed 2 times with 15 mL PBS 1×, and harvested by trypsinization. Cells were collected in a 15 mL tube by centrifugation at 1000 rpm for 5 min. The supernatant was discarded and cells were washed 3 times with PBS 1×. The cell pellet was transferred to an Eppendorf tube and 500 mL of PBS 1× were added. Cells were centrifuged 5 min. at 3000 rpm in an Eppendorf Microfuge. The supernatant was removed and cells were then lysed in 50-150 ml of lysis buffer (50 mM Tris pH 7.5, 150 mM NaCl, 1% NP-40, 0.5% sodium deoxycholate), containing protease inhibitors (1 mg/ml pepstatin, 1 mg/ml leupeptin; 1 mg/ml benzamidine; 0.2 mM PMSF) and incubated 5 min. on ice. The cell lysates were then centrifuged at 14,000×g for 10 min. at 4 C. The protein concentration of the supernatants was determined by the DC Protein assay (BioRad); samples were subsequently stored at −80° C. until ready for analysis.

2. Two Dimensional Gel Electrophoresis of Cell Extracts

Total cell lysates were thawed and then incubated with 1 U/mL DNAse I, 5 mM MgCl2 (final concentration) for 2 hr. on ice. Their protein concentration was determined using the RC DC protein assay kit from BIORAD according to manufacturer's instructions (BioRad Laboratories, Hercules, Calif., USA) (see also Lowry et al., J. Biol. Chem. 193: 265-275, 1951). Finally, urea was added to the cell lysates to obtain a final concentration of 8M. Equivalent amounts of proteins (250 mg) from total cell extracts from sensitive and multidrug resistant cells were analyzed by two-dimensional (2D) gel electrophoresis and visualized by silver staining. This allowed resolution of protein samples according to differences in their isoelectric points in the first dimension and molecular masses in the second dimension. For the first dimension, isoelectric focusing (IEF) was achieved using immobilized pH gradient gel (IPG) strips (pH 4-7, 24 cm, Amersham Pharmacia Biotech, Piscataway, N.J., USA). Briefly, 24 cm strips were rehydrated in a ceramic strip holder in 450 ml rehydration buffer (8 M urea, 2% (w/v) CHAPS, 0.5% (v/v) IPG buffer and 0.0125% bromophenol blue) containing the protein samples for 15 hr. at 30 volts. Electrode pads were then placed over each electrode and the proteins separated on an IPgp 1hor unit using the following program: 24 cm strips (pH 4-7): −500V for 500 Vh, −1000V for 1000 Vh, −8000V for 32000 Vh

Upon completion of IEF, strips were then slightly rinsed with water and equilibrated in 1% DTT in equilibration buffer (50 mM Tris/HCl, pH 8.8, 6 M urea, 30% glycerol, 2% (w/v) SDS and 0.0125% bromophenol blue) for 15 min, followed by 4% iodoacetamide in equilibration buffer for 15 min.

For the second dimension, the above isoelectric strips were subject to sodium dodecyl sulfate-polyacrylamide gel electrophoresis (SDS-PAGE) using a 12.5% gel, according to the method of Laemmli (Laemmli U.K., Nature 227:680-685, 1970). Molecular weight markers were loaded onto a 2×3 mm filter paper and placed at one end of the strip. The strip and molecular weight marker filter were then sealed onto the polyacrylamide gel with a 0.5% agarose solution in running buffer. The gels were run at constant current (5 mA/gel) for 30 min., and then the current was increased to reach 10 mA/gel for 6 hr.

Two-dimensional gels were fixed in 40% (v/v) methanol, 10% (v/v) acid acetic solution for 24 h at room temperature and then silver stained. Briefly, gels were incubated in 750 mL of a sensitizing solution (30% EtOH, 10 mM potassium tetrathionate, 500 mM potassium acetate in nanopure water) for 40 min., then washed 6 times with 750 mL of nanopure water, incubated 30 min. in 750 mL of a staining solution (12.5 mM silver nitrate in nanopure water), washed again 15 sec. in 750 mL of nanopure water and developed in 750 mL of developing solution (250 mM potassium carbonate, 0.00125% (w/v) sodium thiosulfate, 0.01% formaldehyde in nanopure water). The development of the gels was stopped when the desired intensity of staining was reached by transferring the gels in the stopping solution (300 mM Tris, 2% acetic acid in nanopure water). The 2D maps of total cell extracts were compared by using ImageMaster 2D Elite software (Amercham Pharmacia Biotech) and checked manually.

3. Mass Spectrometry Analysis of Peptides from Proteins-of-Interest

Spot of interest was excised with a clean (clean; acid washed) razor blade and cut into small pieces on a clean glass plate and transfer into a 200 μl PCR tube (MeOH treated). The gel pieces were mixed with 50 μl destainer A and 50 μl destainer B (provided with SilverQuest kit, Life Technologies) (or 100 μl of the destainers premix prepared fresh) and incubated for 15 min at room temperature without agitation. The destaining solution was removed using a capillary tip. Water was added to the gel pieces, mix and incubate 10 min at room temperature. The latter step was repeated three times. The gel pieces were then dehydrated in 100 μl 100% methanol for 5 min. at room temperature, followed by rehydration in 30% methanol/water for 5 min. Gel pieces were then washed 2 times in water for 10 min. and 2 times in 25 mM Ambic (ammonium bicarbonate), 30% (v/v) acetonitrile for 10 min.

After complete drying in a speed vac for 20 min., tryptic digestion of the destained and washed gel pieces was performed by adding ˜1 volume of trypsin solution (130 ng of trypsin (Roche Diagnostics, Laval, Qc, Canada) in 25 mM ammonium bicarbonate, 5 mM CaCl2) to 1 volume of gel pieces and samples left on ice for 45 min. Fresh digestion buffer was added and digestion allowed to proceed for 15-16 hrs at 37° C. Digested peptides were extracted with acetonitrile for 15 min. at room temperature with shaking. The gel pieces/solvent were sonicated 5 min. and re-extracted with 5% formic acid: 50% acetonitrile:45% water freshly prepared. The extraction step was repeated several times and the collected material combined and lyophilized to dryness. The extracted peptides were resuspend in 5% methanol with 0.2% trifluroacetic acid then loaded on an equilibrated C18 bed (Ziptip from Millipore, Bedford, Mass., USA). The loaded Ziptip was washed with 5% acetonitrile containing 0.2% TFA and then eluted in 10 ml of 60% acetonitrile. Eluted peptide solution was dried and analyzed using MALDI mass spectroscopy (Mann M, et al. Ann. Rev. Biochem.70: 437-473, 2001). The resulting peptides list were analyzed using the sequence database search shareware software program ProFound™ (http://www.proteomics.con/prowl-cgi/Profound.exe) to obtain protein identity. PROFOUND was used to search public databases for protein sequences (e.g., non-redundant collection of sequences at the US National Center for Biotechnology Information (NCBInr)). The NCBInr database contains translated protein sequences from the entire collection of DNA sequences kept at Genbank, and also the protein sequences in the PDB, SWISS-PROT and PIR databases.

### Example 4

### Detection of Cell Marker Expression Levels using an Antibody Microarray

1. Antibody Microarray Production

An antibody microarray is used to identify the expression levels of cell markers in a cancer cell sample. Derivatized glass slides are obtained commercially from TeleChem International. Antibodies are printed onto the slide using a BioRobotics Microgrid™ Arrayer (BioTek Instruments, Inc., Winooski, Vt.). Antibodies are obtained commercially from, e.g., BD Biosciences (Palo Alto, Calif.). After antibodies are printed onto the slide, aldehydes or other reactive groups that did not react to an antibody during the spotting procedure are quenched with in a TBS (10 mM Tris-HCl, pH 7.5, 10 mM NaCl) buffer wash containing 10% BSA for 1 hr. Excess BSA is removed with two TBS washes for 5 min.

2. Cell Marker Labeling

Cell markers are isolated from 10⁷-10⁸ cells when using cell lines or 50-100 mg of patient tissue. Cells or tissues are suspended in 50 ml of Tris/EDTA Buffer (pH 7.4) with 0.1% Tween 20 and 145 μl of 1.4 mg/ml PMSF. The suspended sample is kept on ice. Cell lysis is accomplished by gentle homogenization with a dounce. The suspension is centrifuged at 4,000-5,000 g for 5 min. and the suspension is placed on ice.

Once protein isolation is complete, cell markers are labeled using manufacturer's protocols and solutions (TeleChem International, Inc.). Briefly, 1 mg of the protein is dissolve 100 ml of PBS in a reaction tube. 20 ml of reaction solution A is added to the protein reaction tube. The reactive dye ArrayIt® Green540 and ArrayIt® Red640 stock are prepared just prior to starting the reaction. The dye tubes are then spiked with 25 ml of solution B. The mixture is mixed to dissolve the solution. Ten milliliters of the reactive dye solution is combined with the protein reaction tube with gentle vortexing. The labeling reaction is incubated at room temperature for 1 hr. in the dark. While the reaction mixture is being incubated, two purification columns supplied by the manufacturer are prepared. The columns are gently tapped to insure that all the gel is at the bottom of the column. The column gel is hydrated by adding 0.8 ml of solution C to each column and vigorous vortexing for about 5 sec. Air bubbles are removed by tapping the bottoms of the columns sharply. The columns are stored at room temperature for 30 min., and then drained of excess fluid. The dye labeling reaction is stopped by incubating the reaction mixture with Buffer D. Excess label is removed by transferring the protein reaction to two purification columns and spinning the columns at 750 g for 2 min. The labeled proteins are then ready for incubation with the antibody microarray.

2. Incubation of the Labeled Cell Markers with the Antibody Microarray

The antibody microarray is brought into contact with a cancer cell sample. The cell markers are diluted in PBST (Phosphate Buffered Saline with 0.1% Tween20) to a concentration of 10 μg/ml. The slide is incubated with 0.55 ml of the cell marker solution at room temperature overnight in a PC500 CoverWell incubation chamber (Grace Biolabs, Bend, Oreg.). The microarray is washed three times in PBST at room temperature for 5 min. per wash to remove excess proteins that did not absorb or bind to the antibodies. The slides are then rinsed with PBS twice and centrifuged for I min. at 200 g. The signal is detected with a TECAN LS300, Alpha Innotech AlphaArray 7000MP (Perkin-Elmer Corp.)

3. Statistical Determination of Protein Expression Levels

As with the nucleic acid focused microarray, the slides are analyzed using QuantArray® software according to the following procedures. All analysis of the results subtracts the spot background values for Cy5 and Cy3 from the experimental results. Spots with lower signal ratio to noise lower than 1.5 should be discarded. Normalization of the ratios with the spike positive control (Arabidopsis thaliana) allows a ratio equal to one for that control on each slide. The slides on which the negative and/or positive controls do not work are discarded. Also, slides are discarded when they show high background and different mean no offset correction as determined by ArrayStat software. The calculation of means for each target requires at least six different experiments (including two reciprocal labeling reactions), each experiment uses different total RNA preparations. Statistical analyses are accomplished with the ArrayStat 1.0 (Imaging Research Inc.). A log transformation of the ratio data is followed by a Student T test for two independent conditions using a proportional model without offsets at a p<0.05 threshold. Significant increases (ratio Cy5/Cy3 higher than 1.5) or decreases (ratio Cy5/Cy3 lower than 0.5) are significant if the p value was lower than 0.05.

### Equivalents

Those skilled in the art will recognize, or be able to ascertain, using no more than routine experimentation, numerous equivalents to the specific compositions and procedures described herein. Such equivalents are considered to be within the scope of this invention, and are covered by the following claims.

1. A method of diagnosing chemotherapeutic drug resistance in a cancer cell sample, comprising:
(a) providing a focused microarray, the microarray having a plurality of nucleic acid capture probes, wherein each capture probe is complementary to probes corresponding to a marker gene selected from the group consisting of Pgp, BCRP, P53, annexin 1, UCHL-1, ezrin, HnRNP, E-FABP, “similar to stratafin”, HSP27, SOD, γ-actin, vimentin, HSC70, galectin-1, prosolin, β-tubulin, GST-P1, α-enolase, HSP90, HSP60, B23, PDI/ER-60 precursor, FAS, Rad23 homolog β, α-tubulin, MRP1, keratin type II, ATP synthase δ, tropomyosin, prohibitin, calumenin, 5C5-2, SLC9A3R1, pyrophosphatase, DADEH1, EIF-4B, APRT, LRT/MVP, MB-COMT, EF2, PDI, BIP, and thioredoxine peroxidase 1, and wherein the focused microarray does not include a nucleic acid capture probe complementary to probes corresponding to a cellular marker gene selected from the group consisting of Ki67, estrogen receptor α, estrogen receptor β, Bcl-2, cathepsin β, cathepsin δ, keratin 19, topoisomerase type II α, P53, and GAPDH;
(b) detecting a level of expression in the cancer cell sample of a plurality of probes corresponding to marker genes that are complementary to the plurality of nucleic acid capture probes on the focused microarray; and
(c) comparing the level of expression of the plurality of marker genes in the cancer cell sample to the level of expression of the plurality of marker genes in a non-drug resistant cancer cell of the same tissue type,
wherein the cancer cell is drug resistant if the level of expression of one or more of the plurality of marker genes in the cancer cell sample is greater than the level of expression of the same marker gene(s) in the non-drug resistant cancer cell of the same tissue type.

2. The method according to 1, wherein the microarray has a plurality of nucleic acid capture probes selected from the group consisting of annexin 1, galectin-1, α-enolase, MRP1, PDI/ER-60 precursor, keratin type II, calumenin, prohibitin, and Pgp.

3. The method according to 2, wherein the plurality of nucleic acid capture probes is at least two.

4. The method according to 2, wherein the plurality of nucleic acid capture probes is at least three.

5. The method according to 2, wherein the plurality of nucleic acid capture probes is at least four.

6. The method according to 2, wherein the plurality of nucleic acid capture probes is at least five.

7. The method according to 1, wherein the cancer cell is drug resistant if the level of expression of two or more of the plurality of marker genes in the cancer cell sample is greater than the level of expression of the same marker gene(s) in the non-drug resistant cancer cell of the same tissue type.

8. The method according to 1, wherein the cancer cell is drug resistant if the level of expression of three or more of the plurality of marker genes in the cancer cell sample is greater than the level of expression of the same marker gene(s) in the non-drug resistant cancer cell of the same tissue type.

9. The method according to 1, wherein the cancer cell is drug resistant if the level of expression of four or more of the plurality of marker genes in the cancer cell sample is greater than the level of expression of the same marker gene(s) in the non-drug resistant cancer cell of the same tissue type.

10. The method according to 1, wherein the level of expression of annexin-1 is detected and the cancer cell is from breast tissue.

11. The method according to 1, wherein the level of expression of keratin type II is detected and the cancer cell is from lung tissue.

12. A method of diagnosing chemotherapeutic drug resistance in a cancer cell sample, comprising:
(a) providing a focused microarray, the microarray having a plurality of at least five nucleic acid capture probes, wherein each capture probe is complementary to probes corresponding to a marker gene selected from the group consisting of Pgp, BCRP, P53, annexin 1, UCHL-1, ezrin, HnRNP, E-FABP, “similar to stratafin”, HSP27, SOD, γ-actin, vimentin, HSC70, galectin-1, prosolin, β-tubulin, GST-P1, α-enolase, HSP90, HSP60, B23, PDI/ER-60 precursor, FAS, Rad23 homolog β, α-tubulin, MRP1, keratin type II, ATP synthase δ, tropomyosin, prohibitin, calumenin, 5C5-2, SLC9A3R1, pyrophosphatase, DADEH1, EIF-4B, APRT, LRT/MVP, MB-COMT, EF2, PDI, BIP, and thioredoxine peroxidase 1;
(b) detecting, a level of expression in the cancer cell sample of a plurality of probes corresponding to marker genes that are complementary to the plurality of nucleic acid capture probes on the focused microarray; and
(c) comparing the level of expression of the plurality of marker genes in the cancer cell sample to the level of expression of the plurality of marker genes in a non-drug resistant cancer cell of the same tissue type,
wherein the cancer cell sample is drug resistant if the level of expression of one or more of the plurality of marker genes in the cancer cell sample is greater than the level of expression of the same marker gene(s) in the non-drug resistant cancer cell of the same tissue type.

13. The method according to 12, wherein the microarray has a plurality of nucleic acid capture probes selected from the group consisting of annexin 1, galectin-1, α-enolase, MRP1, PDI/ER-60 precursor, keratin type II, calumenin, prohibitin, and Pgp.

14. The method according to 12, wherein the plurality of nucleic acid capture probes is at least six.

15. The method according to 12, wherein the plurality of nucleic acid capture probes is at least seven.

16. The method according to 12, wherein the plurality of nucleic acid capture probes is at least eight.

17. The method according to 12, wherein the cancer cell is drug resistant if the level of expression of two or more of the plurality of marker genes in the cancer cell sample is greater than the level of expression of the same marker gene(s) in the non-drug resistant cancer cell of the same tissue type.

18. The method according to 12, wherein the cancer cell is drug resistant if the level of expression of three or more of the plurality of marker genes in the cancer cell sample is greater than the level of expression of the same marker gene(s) in the non-drug resistant cancer cell of the same tissue type.

19. The method according to 12, wherein the cancer cell is drug resistant if the level of expression of four or more of the plurality of marker genes in the cancer cell sample is greater than the level of expression of the same marker gene(s) in the non-drug resistant cancer cell of the same tissue type.

20. The method according to 12, wherein the level of expression of annexin-1 is detected and the cancer cell is from breast tissue.

21. The method according to 12, wherein the level of expression of keratin type II is detected and the cancer cell is from lung tissue.

22. A method of diagnosing chemotherapeutic drug resistance in a breast cancer cell, comprising:
(a) selecting a plurality of at least four marker genes selected from the group consisting of Pgp, BCRP, L-plastin, annexin 1, ezrin, HnRNP, E-FABP, SOD, γ-actin, vimentin, HSC70, KAP-1, prosolin, β-tubulin, GST-P1, stratafin, HSP90, nucleophosmin, PDI, MRP1, ATP synthase β, ATP synthase δ, tropomyosin, prohibitin, 5C5-2, HSP27, HSP60, tropomyosin, calumenin, and thioredoxine peroxidase 1;
(b) detecting a level of expression in the breast cancer cell sample of the plurality of marker genes; and
(c) comparing the level of expression of the plurality of marker genes in the breast cancer cell sample to the level of expression of the plurality of marker genes in a non-drug resistant cancer cell of the same tissue type,
wherein the breast cancer cell sample is drug resistant if the level of expression of a plurality of the marker genes in the breast cancer cell sample is greater than the level of expression of the same marker genes in the non-drug resistant breast cancer cell.

23. The method according to 22, wherein the plurality of marker genes selected is at least five, and a higher level of expression of a plurality of at least three marker genes in the breast cancer cell sample compared to the non-resistant breast cancer cell indicates that the breast cancer cell sample is drug resistant.

24. The method according to 22, wherein the plurality of marker genes selected is at least six, and a higher level of expression of a plurality of at least four marker genes in the breast cancer cell sample compared to the non-resistant breast cancer cell indicates that the breast cancer cell sample is drug resistant.

25. The method according to 22, wherein the plurality of marker genes selected is at least eight and a higher level of expression of a plurality of at least six marker genes in the breast cancer cell sample compared to the non-resistant breast cancer cell indicates that the breast cancer cell sample is drug resistant.

26. The method according to 22, wherein the level of expression of cancer cell markers is detected using capture probes attached to a solid support.

27. The method according to 22, wherein the plurality of at least four marker genes is selected from the group consisting of prohibitin, Pgp, calumenin, tropomyosin, L-plastin, stratafin, and prefoldin subunit 1.

28. The method according to 27, wherein a higher level of expression of a plurality of at least three marker genes in the breast cancer cell sample compared to the non-resistant breast cancer cell indicates that the breast cancer cell sample is drug resistant.

29. The method according to 22, wherein a higher level of expression of annexin-1 in the breast cancer cell sample compared to the non-resistant breast cancer cell indicates that the breast cancer cell sample is drug resistant.

30. A method for diagnosing chemotherapeutic drug resistance in a lung cancer cell, comprising:
(a) selecting a plurality of at least four marker genes selected from the group consisting of Pgp, annexin 1, γ-actin, vimentin, galectin-1, β-tubulin, α-enolase, HSP90, nucleophosmin, MRP1, keratin type II, ATP synthase δ, tropomyosin, prohibitin, calumenin, 5C5-2, and SLC9A3R1;
(b) detecting a level of expression in the lung cancer cell sample of the plurality of marker genes; and
(c) comparing the level of expression of the plurality of marker genes in the lung cancer cell sample to the level of expression of the plurality of marker genes in a non-drug resistant cancer cell of the same tissue type,
wherein the lung cancer cell sample is drug resistant if the level of expression of one or more of the plurality of marker genes in the lung cancer cell sample is greater than the level of expression of the same marker gene(s) in the non-drug resistant cancer cell of the same tissue type.

31. The method according to claim 30, wherein the plurality of nucleic acid capture probes is at least five, and a higher level of expression of a plurality of at least three marker genes in the lung cancer cell sample compared to the non-resistant lung cancer cell indicates that the lung cancer cell sample is drug resistant.

32. The method according to 30, wherein the plurality of marker genes selected is at least six, and a higher level of expression of a plurality of at least four marker genes in the lung cancer cell sample compared to the non-resistant lung cancer cell indicates that the lung cancer cell sample is drug resistant.

33. The method according to 30, wherein the plurality of marker genes selected is at least eight and a higher level of expression of a plurality of at least six marker genes in the lung cancer cell sample compared to the non-resistant lung cancer cell indicates that the lung cancer cell sample is drug resistant.

34. The method according to claim 30, wherein the level of expression of cancer cell markers is detected using capture probes attached to a solid support.

35. The method according to claim 30, wherein the plurality of at least four marker genes is selected from the group consisting of Pgp, γ-actin, HSP90, calumenin, prohibitin, ATP synthase δ, galectin-1 and keratin type II.

36. The method according to 35, wherein a higher level of expression of a plurality of at least three marker genes in the lung cancer cell sample compared to the non-resistant lung cancer cell indicates that the lung cancer cell sample is drug resistant.

37. The method according to claim 30, wherein a higher level of expression of keratin type II in the lung cancer cell sample compared to the non-resistant lung cancer cell indicates that the lung cancer cell sample is drug resistant.

38. A method for diagnosing chemotherapeutic drug resistance in an ovarian cancer cell, comprising:
(a) selecting a plurality of at least four marker genes selected from the group consisting of Pgp, P53, annexin 1, ezrin, KAP-1, HnRNP, E-FABP, HSP27, SOD, γ-actin, vimentin, HSC70, galectin-1, prosolin, β-tubulin, α-enolase, HSP90, HSP60, nucleophosmin, FAS, Rad23 homolog β, α-tubulin, MRP1, keratin type II, tropomyosin, prohibitin, calumenin, 5C5-2, SLC9A3R1, pyrophosphatase, MB-COMT, EF2, PDI, and PDI/ER 60 precursor protein;
(b) detecting a level of expression in the ovarian cancer cell sample of the plurality of marker genes; and
(c) comparing the level of expression of the plurality of marker genes in the ovarian cancer cell sample to the level of expression of the plurality of marker genes in a non-drug resistant cancer cell of the same tissue type,
wherein the ovarian cancer cell sample is drug resistant if the level of expression of one or more of the plurality of marker genes in the ovarian cancer cell sample is greater than the level of expression of the same marker gene(s) in the non-drug resistant cancer cell of the same tissue type.

39. The method according to claim 38, wherein the plurality of nucleic acid capture probes is at least five, and a higher level of expression of a plurality of at least three marker genes in the ovarian cancer cell sample compared to the non-resistant ovarian cancer cell indicates that the ovarian cancer cell sample is drug resistant.

40. The method according to 38, wherein the plurality of marker genes selected is at least six, and a higher level of expression of a plurality of at least four marker genes in the ovarian cancer cell sample compared to the non-resistant ovarian cancer cell indicates that the ovarian cancer cell sample is drug resistant.

41. The method according to 38, wherein the plurality of marker genes selected is at least eight and a higher level of expression of a plurality of at least six marker genes in the ovarian cancer cell sample compared to the non-resistant ovarian cancer cell indicates that the ovarian cancer cell sample is drug resistant.

42. The method according to claim 38, wherein the level of expression of cancer cell markers is detected using capture probes attached to a solid support.

43. The method according to claim 38, wherein the plurality of at least four marker genes is selected from the group consisting of Pgp, HSP60, prohibitin, galectin-1, nucleophosmin, annexin-1 and calumenin.

44. The method according to 43, wherein a higher level of expression of a plurality of at least three marker genes in the ovarian cancer cell sample compared to the non-resistant ovarian cancer cell indicates that the ovarian cancer cell sample is drug resistant.

45. The method according to claim 38, wherein a higher level of expression of annexin-1 in the ovarian cancer cell sample compared to the non-resistant ovarian cancer cell indicates that the ovarian cancer cell sample is drug resistant.

46. A method of diagnosing chemotherapeutic drug resistance in a breast cancer cell, comprising:
(a) providing a focused microarray as in claim 75, the microarray having a first set and a second set of nucleic acid capture probes, wherein each capture probe detects the expression level of a marker gene, and the first set nucleic acid capture probes detects a plurality of marker genes selected from the group consisting of keratin 19, c-erb P2/HER-2, SLC9A3R1, A-CRAB II, HSC70, prosolin, ezrin, prohibitin, p16INK4a, MYL16, interleukine 18 precursor, prefoldin subunit 1, HSP60, DADEH1, EF2, EIF4B, and PDI, and the second set of nucleic acid capture probes detects a plurality of marker genes selected from the group consisting of cathepsin δ, PDI, and cathepsin β;
(b) detecting a level of expression in the breast cancer cell sample of the first and the second set of marker genes; and
(c) comparing the level of expression of the first and second set of marker genes in the breast cancer cell sample to the level of expression of the first and second set of marker genes in a non-drug resistant breast cancer cell,
wherein the breast cancer cell sample is drug resistant if the level of expression of a plurality of the marker genes of the first and/or second set in the breast cancer cell sample is greater than the level of expression of the same marker genes in the non-drug resistant breast cancer cell.

47. The method according to 46 further comprising:
(d) determining the expression levels in the breast cancer cell sample of housekeeping genes selected from the group consisting of FABP7, DADEH1, EF2, EIF4B, and cathepsin β; and
(e) comparing the levels of expression of the housekeeping genes in the breast cancer cell sample to the levels of expression of the marker genes in the breast cancer cell.

48. The method according to 46, wherein the breast cancer cell is adriamycin resistant if the level of expression of two or more of the first set of marker genes in the cancer cell sample is greater than the level of expression of the same marker gene(s) in the non-adriamycin resistant breast cancer cell.

49. The method according to 46, wherein the breast cancer cell is adriamycin resistant if the level of expression of three or more of the first set of marker genes in the cancer cell sample is greater than the level of expression of the same marker gene(s) in the non-adriamycin resistant breast cancer cell.

50. The method according to 46, wherein the breast cancer cell is adriamycin resistant if the level of expression of four or more of the first set of marker genes in the cancer cell sample is greater than the level of expression of the same marker gene(s) in the non-adriamycin resistant breast cancer cell.

51. The method according to 46, wherein the breast cancer cell is taxol resistant if the level of expression of two or more of the second set of marker genes in the cancer cell sample is greater than the level of expression of the same marker gene(s) in the non-taxol resistant breast cancer cell.

52. The method according to 46, wherein the breast cancer cell is taxol resistant if the level of expression of three or more of the second set of marker genes in the cancer cell sample is greater than the level of expression of the same marker gene(s) in the non-taxol resistant breast cancer cell.

53. The method according to 46, wherein the breast cancer cell is taxol resistant if the level of expression of four or more of the second set of marker genes in the cancer cell sample is greater than the level of expression of the same marker gene(s) in the non-taxol resistant breast cancer cell.

54. The method according to 46, wherein the level of expression of cancer cell markers is detected using capture probes attached to a solid support.

55. A method of diagnosing chemotherapeutic taxol resistance in an ovarian cancer cell, comprising:
(a) providing a focused microarray as in claim 89, the microarray having a plurality of nucleic acid capture probes, wherein each capture probe detects the expression of a marker gene selected from the group consisting of p53, A-CRABP II, KAP-1, HSP60, nucleophosmin, ezrin, prohibitin, and prefoldin subunit 1;
(c) detecting a level of expression in the ovarian cancer cell sample of a plurality of marker genes; and
(d) comparing the level of expression of the plurality of marker genes in the ovarian cancer cell sample to the level of expression of the plurality of marker genes in a taxol sensitive ovarian cancer cell,
wherein the ovarian cancer cell sample is taxol resistant if the level of expression of at least one of the marker genes in the ovarian cancer cell sample is greater than the level of expression of the same marker genes in the taxol sensitive ovarian cancer cell.

56. The method according to 55, wherein the ovarian cancer cell is taxol resistant if the level of expression of two or more of the plurality of marker genes in the cancer cell sample is greater than the level of expression of the same marker gene(s) in the non-taxol resistant ovarian cancer cell.

57. The method according to 55, wherein the ovarian cancer cell is taxol resistant if the level of expression of three or more of the plurality of marker genes in the cancer cell sample is greater than the level of expression of the same marker gene(s) in the non-taxol resistant ovarian cancer cell.

58. The method according to 55, wherein the ovarian cancer cell is taxol resistant if the level of expression of four or more of the plurality of marker genes in the cancer cell sample is greater than the level of expression of the same marker gene(s) in the non-taxol resistant ovarian cancer cell.

59. The method according to 55 further comprising:
(e) determining the expression levels in the ovarian cancer cell sample of housekeeping genes selected from the group consisting of FABP7, DADEH1, EF2, EIF4B, and cathepsin β; and
(f) comparing the levels of expression of the housekeeping genes in the ovarian cancer cell sample to the levels of expression of the marker genes in the ovarian cancer cell.

60. A focused microarray for diagnosis of chemotherapeutic drug resistance comprising:
(a) a plurality of at least five nucleic acid capture probes, wherein each capture probe is complementary to probes corresponding to a marker gene selected from the group consisting of Pgp, BCRP, P53, annexin 1, UCHL-1, ezrin, HnRNP, E-FABP, “similar to stratafin”, HSP27, SOD, γ-actin, vimentin, HSC70, galectin-1, prosolin, β-tubulin, GST-P1, α-enolase, HSP90, HSP60, B23, PDI/ER-60 precursor, FAS, Rad23 homolog β, α-tubulin, MRP1, keratin type II, ATP synthase δ, tropomyosin, prohibitin, calumenin, 5C5-2, SLC9A3R1, pyrophosphatase, DADEH1, EIF-4B, APRT, LRT/MVP, MB-COMT, EF2, PDI, BIP, and thioredoxine peroxidase 1, wherein the focused microarray does not include a nucleic acid capture probe complementary to probes corresponding to a cellular marker gene selected from the group consisting of Ki67, estrogen receptor α, estrogen receptor β, Bcl-2, cathepsin β, cathepsin δ, keratin 19, topoisomerase type II α, P53, and GAPDH; and
(b) a solid support to which the plurality of nucleic acid capture probes is attached at discrete positions.

61. The microarray of claim 60, wherein the plurality of nucleic acid capture probes comprises at least one of the markers selected from the group consisting of annexin 1, galectin-1, HSP27, keratin type II, MRP1, calumenin, prohibitin, and Pgp.

62. The microarray of claim 61, wherein the plurality of nucleic acid capture probes comprises at least two of the markers selected from the group consisting of annexin 1, galectin-1, HSP27, keratin type II, MRP1, calumenin, prohibitin, and Pgp.

63. The microarray of claim 61, wherein the plurality of nucleic acid capture probes comprises at least three of the markers selected from the group consisting of annexin 1, galectin-1, HSP27, keratin type II, MRP1, calumenin, prohibitin, and Pgp.

64. The microarray of claim 61, wherein the plurality of nucleic acid capture probes comprises at least four of the markers selected from the group consisting of annexin 1, galectin-1, HSP27, keratin type II, MRP1, calumenin, prohibitin, and Pgp.

65. The microarray of claim 61, wherein the plurality of nucleic acid capture probes comprises at least five of the markers selected from the group consisting of annexin 1, galectin-1, HSP27, keratin type II, MRP1, calumenin, prohibitin, and Pgp.

66. The microarray of claim 60, wherein the solid support is composed of a material selected from the group consisting of glass, metal alloy, silicon, and nylon.

67. A focused microarray for diagnosis of chemotherapeutic drug resistance in breast cancer comprising:
(a) a plurality of at least four nucleic acid capture probes, wherein each capture probe is complementary to probes corresponding to a marker gene selected from the group consisting of Pgp, BCRP, L-plastin, annexin 1, ezrin, HnRNP, E-FABP, SOD, γ-actin, vimentin, HSC70, KAP-1, prosolin, β-tubulin, GST-P1, stratafin, HSP90, nucleophosmin, PDI, MRP1, ATP synthase β, ATP synthase δ, tropomyosin, prohibitin, 5C5-2, HSP27, HSP60, tropomyosin, calumenin, and thioredoxine peroxidase 1, wherein the focused microarray does not include a nucleic acid capture probe complementary to probes corresponding to a cellular marker gene selected from the group consisting of Ki67, estrogen receptor α, estrogen receptor β, Bcl-2, cathepsin β, cathepsin δ, keratin 19, topoisomerase type II α, P53, and GAPDH; and
(b) a solid support to which the plurality of nucleic acid capture probes is attached at discrete positions.

68. A focused microarray for diagnosis of chemotherapeutic drug resistance in breast cancer comprising:
(a) a plurality of nucleic acid capture probes, wherein each capture probe is complementary to probes corresponding to a marker gene selected from the group consisting of keratin 19, c-erb β2/HER-2, SLC9A3R1, A-CRAB II, cytokeratin 7, HSC70, prosolin, ezrin, prohibitin, p16INK4a, MYL16, interleukine 18 precursor, prefoldin subunit 1, HSP60, DADEH1, EF2, EIF4B, cathepsin B, and PDI; and
(b) a solid support to which the plurality of nucleic acid capture probes is attached at discrete positions.

69. The microarray of claim 68, wherein the plurality of nucleic acid capture probes comprises at least one of the markers selected from the group consisting of cytokeratin 7, HSC70, prosolin, ezrin, prohibitin, p16INK4a, MYL16, interleukine 18 precursor, and prefoldin subunit 1.

70. The microarray of claim 69, wherein the plurality of nucleic acid capture probes comprises at least two of the markers selected from the group consisting of cytokeratin 7, HSC70, prosolin, ezrin, prohibitin, p16INK4a, MYL16, interleukine 18 precursor, and prefoldin subunit 1.

71. The microarray of claim 69, wherein the plurality of nucleic acid capture probes comprises at least three of the markers selected from the group consisting of cytokeratin 7, HSC70, prosolin, ezrin, prohibitin, p16INK4a, MYL16, interleukine 18 precursor, and prefoldin subunit 1.

72. The microarray of claim 69, wherein the plurality of nucleic acid capture probes comprises at least four of the markers selected from the group consisting of cytokeratin 7, HSC70, prosolin, ezrin, prohibitin, p6INK4a, MYL16, interleukine 18 precursor, and prefoldin subunit 1.

73. The microarray of claim 69, wherein the plurality of nucleic acid capture probes comprises at least five of the markers selected from the group consisting of cytokeratin 7, HSC70, prosolin, ezrin, prohibitin, p16INK4a, MYL16, interleukine 18 precursor, and prefoldin subunit 1.

74. The microarray of claim 68, wherein the solid support is composed of a material selected from the group consisting of glass, metal alloy, silicon, and nylon.

75. A focused microarray for diagnosis of chemotherapeutic drug resistance in breast cancer comprising:
(a) a first set of nucleic acid capture probes for determining adriamycin resistance, the set comprising a plurality of nucleic acid capture probes, wherein each capture probe is complementary to probes corresponding to a marker gene selected from the group consisting of cytokeratin 7, HSC70, prosolin, ezrin, prohibitin, p16INK4a, MYL16, interleukine 18 precursor, prefoldin subunit 1, cathepsin β, and PDI;
(b) a second set of nucleic acid capture probes for determining taxol resistance, the set comprising a plurality of nucleic acid capture probes, wherein each capture probe is complementary to probes corresponding to a marker gene selected from the group consisting of cathepsin 6, PDI, and cathepsin β;
(c) a third set of nucleic acid capture probes for identifying a breast tumor, the set comprising a plurality of nucleic acid capture probes, wherein each capture probe is complementary to probes corresponding to a marker gene selected from the group consisting of keratin 19, c-erb β2/HER-2, SLC9A3R1, A-CRAB II;
(d) a fourth set of nucleic acid capture probes, the set comprising a plurality of nucleic acid capture probes, wherein each capture probe is complementary to probes corresponding to a marker gene selected from the group consisting of HSP60, DADEH1, EF2, and EIF4B; and
(e) a solid support to which the nucleic acid capture probes are attached at discrete positions.

76. The microarray of claim 75, wherein the plurality of capture probes of the first set comprises at least three of the markers selected from the group consisting of cytokeratin 7, HSC70, prosolin, ezrin, prohibitin, p16INK4a, MYL16, interleukine 18 precursor, and prefoldin subunit 1.

77. The microarray of claim 75, wherein the plurality of capture probes of the first set comprises at least four of the markers selected from the group consisting of cytokeratin 7, HSC70, prosolin, ezrin, prohibitin, p16INK4a, MYL16, interleukine 18 precursor, and prefoldin subunit 1.

78. The microarray of claim 75, wherein the plurality of capture probes of the second set comprises at least three of the markers selected from the group consisting of cathepsin δ, PDI, and cathepsin β.

79. The microarray of claim 75, wherein the plurality of capture probes of the third set comprises at least three of the markers selected from the group consisting of keratin 19, c-erb β2/HER-2, SLC9A3R1, A-CRAB II.

80. The microarray of claim 75, wherein the plurality of capture probes comprises of the fourth set at least three of the markers selected from the group consisting of HSP60, DADEH1, EF2, and EIF4B.

81. The microarray of claims 76-80, wherein the plurality of capture probes is at least two markers.

82. A focused microarray for diagnosis of chemotherapeutic drug resistance in ovarian cancer comprising:
(a) a plurality of nucleic acid capture probes, wherein each capture probe is complementary to probes corresponding to a marker gene selected from the group consisting of p53, A-CRABP II, KAP-1, HSP60, nucleophosmin, ezrin, prohibitin, prefoldin subunit 1, FABP7, DADEH1, EF2, EIF4B, and cathepsin β; and
(b) a solid support to which the plurality of nucleic acid capture probes is attached at discrete positions.

83. The microarray of claim 82, wherein the plurality of nucleic acid capture probes comprises at least one of the markers selected from the group consisting of p53, A-CRABP II, KAP-1, HSP60, nucleophosmin, ezrin, prohibitin, prefoldin subunit 1, FABP7, DADEH1, EF2, EIF4B, and cathepsin β.

84. The microarray of claim 83, wherein the plurality of nucleic acid capture probes comprises at least two of the markers selected from the group consisting of p53, A-CRABP II, KAP-1, HSP60, nucleophosmin, ezrin, prohibitin, prefoldin subunit 1, FABP7, DADEH1, EF2, EIF4B, and cathepsin β.

85. The microarray of claim 83, wherein the plurality of nucleic acid capture probes comprises at least three of the markers selected from the group consisting of p53, A-CRABP II, KAP-1, HSP60, nucleophosmin, ezrin, prohibitin, prefoldin subunit 1, FABP7, DADEH1, EF2, EIF4B, and cathepsin β.

86. The microarray of claim 83, wherein the plurality of nucleic acid capture probes comprises at least four of the markers selected from the group consisting of p53, A-CRABP II, KAP-1, HSP60, nucleophosmin, ezrin, prohibitin, prefoldin subunit 1, FABP7, DADEH1, EF2, EIF4B, and cathepsin β.

87. The microarray of claim 83, wherein the plurality of nucleic acid capture probes comprises at least five of the markers selected from the group consisting of p53, A-CRABP II, KAP-1, HSP60, nucleophosmin, ezrin, prohibitin, prefoldin subunit 1, FABP7, DADEH1, EF2, EIF4B, and cathepsin β.

88. The microarray of claim 82, wherein the solid support is composed of a material selected from the group consisting of glass, metal alloy, silicon, and nylon.

89. A focused microarray for diagnosis of chemotherapeutic drug resistance in ovarian cancer comprising:
(a) a first set of nucleic acid capture probes for determining taxol and cisplatinum resistance, the set comprising a plurality of nucleic acid capture probes, wherein each capture probe is complementary to probes corresponding to a marker gene selected from the group consisting HSP60, nucleophosmin, ezrin, prohibitin, and cathepsin β;
(b) a second set of nucleic acid capture probes for identifying an ovarian tumor, the set comprising a plurality of nucleic acid capture probes, wherein each capture probe is complementary to probes corresponding to a marker gene selected from the group consisting of p53, A-CRABP II, KAP-1, and prefoldin subunit 1;
(c) a third set of nucleic acid capture probes, the set comprising a plurality of nucleic acid capture probes, wherein each capture probe is complementary to probes corresponding to a marker gene selected from the group consisting of FABP7, DADEH1, EF2, and EIF4B; and
(d) a solid support to which the nucleic acid capture probes are attached at discrete positions.

90. The microarray of claim 89, wherein the plurality of capture probes of the first set comprises at least three of the markers selected from the group consisting of HSP60, nucleophosmin, ezrin, prohibitin, and cathepsin β.

91. The microarray of claim 90, wherein the plurality of capture probes of the first set comprises at least four of the markers selected from the group consisting of HSP60, nucleophosmin, ezrin, prohibitin, and cathepsin β.

92. The microarray of claim 90, wherein the plurality of capture probes of the second set comprises at least three of the markers selected from the group consisting of p53, A-CRABP II, KAP-1, and prefoldin subunit 1.

93. The microarray of claim 90, wherein the plurality of capture probes of the third set comprises at least three of the markers selected from the group consisting of FABP7, DADEH1, EF2, and EIF4B.

94. The microarray of claims 90-93, wherein the plurality of capture probes is at least two markers.

95. A focused microarray for diagnosis of chemotherapeutic drug resistance in lung cancer comprising:
(a) a plurality of at least four nucleic acid capture probes, wherein each capture probe is complementary to probes corresponding to a marker gene selected from the group consisting of Pgp, annexin 1, γ-actin, vimentin, galectin-1, β-tubulin, α-enolase, HSP90, nucleophosmin, MRP1, keratin type II, ATP synthase δ, tropomyosin, prohibitin, calumenin, 5C5-2, and SLC9A3R1; and
(b) a solid support to which the plurality of nucleic acid capture probes is attached at discrete positions.

96. A focused microarray for diagnosis of chemotherapeutic drug resistance in ovarian cancer comprising:
(a) a plurality of at least four nucleic acid capture probes, wherein each capture probe is complementary to probes corresponding to a marker gene selected from the group consisting of Pgp, P53, annexin 1, ezrin, KAP-1, HnRNP, E-FABP, HSP27, SOD, γ-actin, vimentin, HSC70, galectin-1, prosolin, β-tubulin, α-enolase, HSP90, HSP60, nucleophosmin, FAS, Rad23 homolog β, α-tubulin, MRP1, keratin type II, tropomyosin, prohibitin, calumenin, 5C5-2, SLC9A3R1, pyrophosphatase, MB-COMT, EF2, PDI, and PDI/ER 60 precursor protein; and
(b) a solid support to which the plurality of nucleic acid capture probes is attached at discrete positions.

97. A method of diagnosing chemotherapeutic drug resistance in a cancer cell sample, comprising:
(a) providing an antibody microarray, the microarray having a plurality of antibodies affixed to its surface, wherein each antibody binds to a cell marker selected from the group consisting of ezrin, HnRNP, UCHL-1, E-FABP, stratafin, vimentin, galectin-1, GST-P1, α-enolase, NES factor attachment protein γ, PDI/ER-60 precursor, Rad23 homolog β, prosolin, tropomyosin, nucleophosmin and ETF3 subunit 2;
(b) detecting a level of protein expression in the cancer cell sample of the plurality of cell markers; and
(c) comparing the level of protein expression of the plurality of cell markers in the cancer cell sample to the level of protein expression of the plurality of cell markers in a non-drug resistant cancer cell of the same tissue type,
wherein the cancer cell is drug resistant if the level of protein expression of at least one cell marker is greater than the level of protein expression of the cell marker in the non-resistant cancer cell of the same tissue type.

98. The method according to claim 97, wherein the plurality of antibodies affixed to the surface is at least two.

99. The method according to claim 97, wherein the plurality of antibodies affixed to the surface is at least three.

100. The method according to claim 97, wherein the plurality of antibodies affixed to the surface is at least four.

101. The method according to claim 97, wherein the plurality of antibodies affixed to the microarray includes an antibody that binds to at least one of the cell markers selected from the group consisting of prosolin, E-FABP, vimentin, HnRNP, tropomyosin, ezrin, galectin-1, GST-P1, and α-enolase.

102. The method according to claim 101, wherein the plurality of antibodies affixed to the microarray includes an antibody that binds to at least two of the cell markers selected from the group consisting of prosolin, E-FABP, vimentin, HnRNP, tropomyosin, ezrin, galectin-1, GST-P1, and α-enolase.

103. The method according to claim 101, wherein the plurality of antibodies affixed to the microarray includes an antibody that binds to at least three of the cell markers selected from the group consisting of prosolin, E-FABP, vimentin, HnRNP, tropomyosin, ezrin, galectin-1, GST-P1, and α-enolase.

104. The method according to 101, wherein the plurality of antibodies affixed to the microarray includes an antibody that binds to at least four of the cell markers selected from the group consisting of prosolin, E-FABP, vimentin, HnRNP, tropomyosin, ezrin, galectin-1, GST-P1, and α-enolase.

105. The method according to 101, wherein the plurality of antibodies affixed to the microarray includes an antibody that binds to at least five of the cell markers selected from the group consisting of prosolin, E-FABP, vimentin, HnRNP, tropomyosin, ezrin, galectin-1, GST-P1, and α-enolase.

106. The method according to 97, wherein the antibodies affixed to the solid surface are IgG-type.

107. The method according to 97, wherein the cancer cell is drug resistant if the level of protein expression of at least two cell markers is greater than the level of protein expression of the cell markers in the non-resistant cancer cell of the same tissue type.

108. The method according to 97, wherein the cancer cell is drug resistant if the level of protein expression of at least three cell markers is greater than the level of protein expression of the cell markers in the non-resistant cancer cell of the same tissue type.

109. The method according to 97, wherein the cancer cell is drug resistant if the level of protein expression of at least four cell markers is greater than the level of protein expression of the cell markers in the non-resistant cancer cell of the same tissue type.

110. The method according to 97, wherein the cancer cell is drug resistant if the level of protein expression of at least five cell markers is greater than the level of protein expression of the cell markers in the non-resistant cancer cell of the same tissue type.

111. The method according to 97, wherein the cancer cell is drug resistant if the level of protein expression of at least six cell markers is greater than the level of protein expression of the cell markers in the non-resistant cancer cell of the same tissue type.

112. A focused antibody microarray for diagnosis of chemotherapeutic drug resistance comprising:
(a) a plurality of at least three antibodies, wherein each antibody binds to a cell marker selected from the group consisting of ezrin, HnRNP, UCHL-1, E-FABP, stratafin, vimentin, galectin-1, GST-P1, α-enolase, NES factor attachment protein γ, E-FABP, PDI/ER-60 precursor, Rad23 homolog β, prosolin, tropomyosin, nucleophosmin and ETF3 subunit 2, and
(b) a solid support to which the plurality of antibodies is attached at discrete position.

113. The focused microarray of claim 112, wherein the plurality of antibodies is at least four antibodies and each antibody binds to a cell marker.

114. The focused microarray of claim 112, wherein the plurality of antibodies binds to at least one of the cell markers selected from the group consisting of prosolin, E-FABP, vimentin, HnRNP, tropomyosin, ezrin, galectin-1, GST-P1, and α-enolase.

115. The focused microarray of claim 114, wherein the plurality of antibodies binds to at least two of the cell markers selected from the group consisting of prosolin, E-FABP, vimentin, HnRNP, tropomyosin, ezrin, galectin-1, GST-P1, and α-enolase.

116. The focused microarray of claim 114, wherein the plurality of antibodies binds to at least three of the cell markers selected from the group consisting of prosolin, E-FABP, vimentin, HnRNP, tropomyosin, ezrin, galectin-1, GST-P1, and α-enolase.

117. The focused microarray of claim 114, wherein the plurality of antibodies binds to at least four of the cell markers selected from the group consisting of prosolin, E-FABP, vimentin, HnRNP, tropomyosin, ezrin, galectin-1, GST-P1, and α-enolase.

118. The focused microarray of claim 114, wherein the plurality of antibodies binds to at least five of the cell markers selected from the group consisting of prosolin, E-FABP, vimentin, HnRNP, tropomyosin, ezrin, galectin-1, GST-P1, and α-enolase.

119. The focused microarray of claim 112, wherein the antibodies affixed to the solid surface are IgG-type.

120. The focused microarray of claim 112, wherein the solid support is composed of a material selected from the group consisting of glass, metal alloy, silicon, and nylon.

121. A method of diagnosing chemotherapeutic drug resistance in a cancer cell sample, comprising:
(a) selecting a plurality of cell markers selected from the group consisting of ezrin, HnRNP, UCHL-1, E-FABP, stratafin, vimentin, galectin-1, GST-P1, α-enolase, NES factor attachment protein γ, E-FABP, PDI/ER-60 precursor, Rad23 homolog β, prosolin, tropomyosin, nucleophosmin and ETF3 subunit 2;
(b) detecting a level of protein expression in the cancer cell sample of the plurality of cell markers; and
(c) comparing the level of protein expression of the plurality of cell markers in the cancer cell sample to the level of protein expression of the plurality of cell markers in a non-drug resistant cancer cell of the same tissue type,
wherein the cancer cell is drug resistant if the level of protein expression of at least one cell marker is greater than the level of protein expression of the cell marker in the non-resistant cancer cell of the same tissue type.

122. The method according to 121, wherein a plurality of at least three cell markers is detected, and the cancer cell is drug resistant if the level of protein expression of at least two cell markers is greater than the level of protein expression of the cell markers in the non-resistant cancer cell of the same tissue type.

123. The method according to 121, wherein a plurality of at least four cell markers is detected, and the cancer cell is drug resistant if the level of protein expression of at least three cell markers is greater than the level of protein expression of the cell markers in the non-resistant cancer cell of the same tissue type.

124. The method according to 121, wherein a plurality of at least five cell markers is detected, and the cancer cell is drug resistant if the level of protein expression of at least four cell markers is greater than the level of protein expression of the cell markers in the non-resistant cancer cell of the same tissue type.

125. The method according to 121, wherein the level of expression of a cell marker is detected by an antibody.

126. The method according to 121, wherein the cancer cell sample is a breast cancer sample.

127. The method according to 126, wherein the breast cancer cell is drug resistant if the level of protein expression of at least two cell markers is greater than the level of protein expression of the cell markers in a non-resistant breast cancer cell.

128. The method according to 126, wherein the breast cancer cell is drug resistant if the level of protein expression of at least three cell markers is greater than the level of protein expression of the cell markers in a non-resistant breast cancer cell.

129. The method according to 126, wherein the breast cancer cell is drug resistant if the level of protein expression of at least four cell markers is greater than the level of protein expression of the cell markers in a non-resistant breast cancer cell.
